SEQUENCE LISTING (24 sequences)

SEQ ID NO: 1 - RNA, 25 nt, Artificial Sequence
  siRNA specific for PD-L1
aaugcguucagcaaaugccaguagg

SEQ ID NO: 2 - RNA, 19 nt, Artificial Sequence
  siRNA specific for PD-L1
cuaauugucuauugggaaa

SEQ ID NO: 3 - RNA, 19 nt, Artificial Sequence
  siRNA specific for PD-L1
cgacuacaagcgaauuacu

SEQ ID NO: 4 - RNA, 25 nt, Artificial Sequence
  siRNA specific for PD-L1 (sense sequence)
ccuacuggcauuugcugaacgcauu

SEQ ID NO: 5 - RNA, 25 nt, Artificial Sequence
  siRNA specific for PD-L1 (anti-sense sequence)
aaugcguucagcaaaugccaguagg

SEQ ID NO: 6 - RNA, 25 nt, Artificial Sequence
  siRNA specific for PD-1
uuacgucuccuccaaauguguauca

SEQ ID NO: 7 - protein, 290 aa, Homo sapiens
  MISC_FEATURE: PD-L1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 8 - DNA, 3691 bp, Homo sapiens
  misc_feature: CD274 encoding PD-L1
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgcatttactgtcacggttcccaaggacctatatgtggtagagtatggtagcaatatgacaattgaatgcaaattcccagtagaaaaacaattagacctggctgcactaattgtctattgggaaatggaggataagaacattattcaatttgtgcatggagaggaagacctgaaggttcagcatagtagctacagacagagggcccggctgttgaaggaccagctctccctgggaaatgctgcacttcagatcacagatgtgaaattgcaggatgcaggggtgtaccgctgcatgatcagctatggtggtgccgactacaagcgaattactgtgaaagtcaatgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa

SEQ ID NO: 9 - protein, 288 aa, Homo sapiens
  MISC_FEATURE: PD-1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 10 - DNA, 2115 bp, Homo sapiens
  misc_feature: PDCD1 encoding PD-1
agtttcccttccgctcacctccgcctgagcagtggagaaggcggcactctggtggggctgctccaggcatgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctgtggactatggggagctggatttccagtggcgagagaagaccccggagccccccgtgccctgtgtccctgagcagacggagtatgccaccattgtctttcctagcggaatgggcacctcatcccccgcccgcaggggctcagctgacggccctcggagtgcccagccactgaggcctgaggatggacactgctcttggcccctctgaccggcttccttggccaccagtgttctgcagaccctccaccatgagcccgggtcagcgcatttcctcaggagaagcaggcagggtgcaggccattgcaggccgtccaggggctgagctgcctgggggcgaccggggctccagcctgcacctgcaccaggcacagccccaccacaggactcatgtctcaatgcccacagtgagcccaggcagcaggtgtcaccgtcccctacagggagggccagatgcagtcactgcttcaggtcctgccagcacagagctgcctgcgtccagctccctgaatctctgctgctgctgctgctgctgctgctgctgcctgcggcccggggctgaaggcgccgtggccctgcctgacgccccggagcctcctgcctgaacttgggggctggttggagatggccttggagcagccaaggtgcccctggcagtggcatcccgaaacgccctggacgcagggcccaagactgggcacaggagtgggaggtacatggggctggggactccccaggagttatctgctccctgcaggcctagagaagtttcagggaaggtcagaagagctcctggctgtggtgggcagggcaggaaacccctccacctttacacatgcccaggcagcacctcaggccctttgtggggcagggaagctgaggcagtaagcgggcaggcagagctggaggcctttcaggcccagccagcactctggcctcctgccgccgcattccaccccagcccctcacaccactcgggagagggacatcctacggtcccaaggtcaggagggcagggctggggttgactcaggcccctcccagctgtggccacctgggtgttgggagggcagaagtgcaggcacctagggccccccatgtgcccaccctgggagctctccttggaacccattcctgaaattatttaaaggggttggccgggctcccaccagggcctgggtgggaaggtacaggcgttcccccggggcctagtacccccgccgtggcctatccactcctcacatccacacactgcacccccactcctggggcagggccaccagcatccaggcggccagcaggcacctgagtggctgggacaagggatcccccttccctgtggttctattatattataattataattaaatatgagagcatgctaaggaaaa

SEQ ID NO: 11 - protein, 1368 aa, Streptococcus pyogenes [sequence omitted]

SEQ ID NO: 12 - protein, 1368 aa, Streptococcus pyogenes
  MISC_FEATURE: S. Pyogenes Cas9 D10A
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 13 - DNA, 20 bp, Artificial Sequence
  PDCD1 CRISPR guide RNA target sequence 1
tgacgttacctcgtgcggcc

SEQ ID NO: 14 - DNA, 20 bp, Artificial Sequence
  PDCD1 CRISPR guide RNA target sequence 2
cacgaagctctccgatgtgt

SEQ ID NO: 15 - DNA, 20 bp, Artificial Sequence
  PDCD1 CRISPR guide RNA target sequence 3
gcgtgacttccacatgagcg

SEQ ID NO: 16 - DNA, 20 bp, Artificial Sequence
  PDCD1 CRISPR guide RNA target sequence 4
ttggaactggccggctggcc

SEQ ID NO: 17 - DNA, 20 bp, Artificial Sequence
  PDCD1 CRISPR guide RNA target sequence 5
gtggcatactccgtctgctc

SEQ ID NO: 18 - DNA, 20 bp, Artificial Sequence
  PDCD1 CRISPR guide RNA target sequence 6
gatgaggtgcccattccgct

SEQ ID NO: 19 - DNA, 20 bp, Artificial Sequence
  CD274 CRISPR guide RNA target sequence 1
taccgctgcatgatcagcta

SEQ ID NO: 20 - DNA, 20 bp, Artificial Sequence
  CD274 CRISPR guide RNA target sequence 2
agctactatgctgaaccttc

SEQ ID NO: 21 - DNA, 20 bp, Artificial Sequence
  CD274 CRISPR guide RNA target sequence 3
ggatgaccaattcagctgta

SEQ ID NO: 22 - DNA, 20 bp, Artificial Sequence
  CD274 CRISPR guide RNA target sequence 4
accccaaggccgaagtcatc

SEQ ID NO: 23 - DNA, 20 bp, Artificial Sequence
  CD274 CRISPR guide RNA target sequence 5
tctttatattcatgacctac

SEQ ID NO: 24 - DNA, 20 bp, Artificial Sequence
  CD274 CRISPR guide RNA target sequence 6
accgttcagcaaatgccagt

(19) United States
(12) Patent Application Publication
(10) Pub. No.: US 2018/0161368 A1
(43) Pub. Date: Jun. 14, 2018

(54) COMPOSITION AND METHODS FOR REGULATING INHIBITORY INTERACTIONS IN GENETICALLY ENGINEERED CELLS

(71) Applicant: Juno Therapeutics, Inc., Seattle, WA (US)

(72) Inventor: Valerie ODEGARD, Seattle, WA (US)

(73) Assignee: Juno Therapeutics, Inc., Seattle, WA (US)

(21) Appl. No.: 15/575,330

(22) PCT Filed: May 27, 2016
(86) PCT No.: PCT/US2016/034873

§ 371 Date: Nov. 17, 2017

Related U.S. Application Data
(60) Provisional application No. 62/168,721, filed on May 29, 2015.
(60) Provisional application No. 62/244,132, filed on Oct. 20, 2015.

(51) Int. Cl. A61K 35/17; C12N 15/11; C12N 15/113; A61P 35/00; C12N 15/86

(52) U.S. Cl. CPC A61K 35/17; C12N 15/111; C12N 15/1138; A61P 35/00; C12N 15/86; C07K 2319/71; C12N 2310/141; C12N 2320/31; C12N 2310/20; C07K 2319/81; C12N 2310/11

(57) ABSTRACT

Provided are engineered cells for adoptive therapy, including T cells. Also provided are methods and compositions for engineering and producing the cells, compositions containing the cells, and method for their administration to subjects. In some embodiments, the cells, such as T cells, contain genetically engineered antigen receptors that specifically bind to antigens, such as a chimeric antigen receptor (CAR). In some embodiments, the cells, such as a CAR-expressing T cell, contains an agent that is capable of reducing an inhibitory effect by repressing and/or disrupting a gene in an engineered cell, such as a gene involved in inhibiting the immune response. In some embodiments, features of the cells and methods provide for increased or improved activity, efficacy and/or persistence.

### CROSS-REFERENCE TO RELATED APPLICATIONS

This application claims priority from U.S. provisional application No. 62/168,721 filed May 29, 2015, entitled “Composition and Methods for Regulating Inhibitory Interactions in Genetically Engineered Cells” and from U.S. provisional application No. 62/244,132, filed Oct. 20, 2015, entitled “Composition and Methods for Regulating Inhibitory Interactions in Genetically Engineered Cells,” the contents of each which are incorporated by reference in their entirety.

### INCORPORATION BY REFERENCE OF SEQUENCE LISTING

The present application is being filed with a Sequence Listing in electronic format. The Sequence Listing is provided as a file entitled 735042002440seqlist.txt, created May 27, 2016, which is 41 kilobytes in size. The information in electronic format of the Sequence Listing is incorporated by reference in its entirety.

### FIELD

The present disclosure relates in some aspect to engineered cells for adoptive therapy, including T cells. In some aspects, the disclosure further relates to methods and compositions for engineering and producing the cells, compositions containing the cells, and method for their administration to subjects. In some embodiments, the cells, such as T cells, contain genetically engineered antigen receptors that specifically bind to antigens, such as a chimeric antigen receptor (CAR). In some embodiments, the cells, such as a CAR-expressing T cell, contains an agent that is capable of reducing an inhibitory effect by repressing and/or disrupting a gene in an engineered cell, such as a gene involved in inhibiting the immune response. In some embodiments, features of the cells and methods provide for increased or improved activity, efficacy and/or persistence.

### BACKGROUND

Various strategies are available for producing and administering engineered cells for adoptive therapy. For example, strategies are available for engineering immune cells expressing genetically engineered antigen receptors, such as CARs, and for suppression or repression of gene expression in the cells. Improved strategies are needed to improve efficacy of the cells, for example, by avoiding suppression of effector functions and improving the activity and/or survival of the cells upon administration to subjects. Provided are methods, cells, compositions, kits, and systems that meet such needs.

### SUMMARY

Provided are methods for producing or generating cells expressing genetically engineered (recombinant) cell surface receptors, such as for use in adoptive cell therapy, for example, to treat diseases and/or conditions in the subjects. Also provided are cells, compositions, and articles of manufacture for use in such methods. The compositions and cells generally include an agent that reduces, or is capable of effecting reduction of, expression of PD-L1 and/or PD-1. In some embodiments, the agent is or comprises an inhibitory nucleic acid molecule, such as one that is complementary to, targets, inhibits and/or binds a gene or nucleic acid encoding PD-L1 or PD-1. In some embodiments, the agent is or comprises a complex comprising a ribonucleoprotein (RNP) complex that includes Cas9, e.g. in some cases an enzymatically inactive Cas9, and a gRNA targeting a gene encoding PD-L1 or PD-1. Also provided are methods for administering to subjects the provided cells expressing genetically engineered (recombinant) cell surface receptors, such as produced by the methods, for example, for adoptive cell therapy to treat diseases and/or conditions in the subjects.

In some embodiments, provided are cells that contain a nucleic acid molecule encoding a genetically engineered antigen receptor, such as a chimeric antigen receptor (CAR) and a nucleic acid molecule that is or includes or encodes an agent that reduces, or is capable of effecting reduction of, expression of PD-L1. In some embodiments, the recombinant receptors are genetically engineered antigen receptors, such as functional non-TCR antigen receptors, e.g., chimeric antigen receptors (CARs) and other recombinant antigen receptors such as transgenic T cell receptors (TCRs). Also among the receptors are other recombinant chimeric receptors, such as those containing an extracellular portion that specifically binds to a ligand or receptor or other binding partner and an intracellular signaling portion, such as the intracellular signaling portion of a CAR. Provided are methods for administering to subjects cells expressing genetically engineered (recombinant) cell surface receptors in adoptive cell therapy, for example, to treat diseases and/or conditions in the subjects.

In some of any such embodiments, an engineered T cell contains a genetically engineered antigen receptor that specifically binds to an antigen; and an agent that reduces, or is capable of effecting reduction of, expression of PD-L1. In some embodiments, the agent comprises an inhibitory nucleic acid molecule, such as one that is complementary to, targets, inhibits and/or binds a gene or other nucleic acid encoding PD-L1 and/or a gene or other nucleic acid encoding PD-L1 (e.g. CD274 gene). In some of any such embodiments, the inhibitory nucleic acid molecule includes an RNA interfering agent. In some of any such embodiments, the inhibitory nucleic acid is or contains or encodes a small interfering RNA (siRNA), a microRNA-adapted shRNA, a short hairpin RNA (shRNA), a hairpin siRNA, a precursor microRNA (pre-miRNA) or a microRNA (miRNA).

In some of any such embodiments, the inhibitory nucleic acid molecule contains a sequence complementary to a PD-L1-encoding nucleic acid. In some of any such embodiments, the inhibitory nucleic acid molecule contains an antisense oligonucleotide complementary to a PD-L1-encoding nucleic acid.

In some embodiments, the agent comprises a gRNA having a targeting domain that is complementary with a target domain of the gene encoding PD-L1 in combination with a Cas9 molecule, such as an enzymatically inactive Cas9 (e.g. eiCas9) for reducing or repressing gene expression. In some embodiments, the agent comprises nucleic acid molecules encoding the at least one gRNA and/or the Cas9 molecule. In some embodiments, the agent comprises at least one complex of the Cas9 molecule and a gRNA having a targeting domain that is complementary with a target domain of the PD-L1 gene.

In some of any such embodiments, a genetically engineered T cell contains a genetically engineered antigen receptor that specifically binds to an antigen; and a disrupted PD-L1-encoding gene, an agent for disruption of a PD-L1-encoding gene, and/or disruption of a gene encoding PD-L1. In some of any such embodiments, the disruption of the gene is mediated by a gene editing nuclease, a zinc finger nuclease (ZFN), a clustered regularly interspaced short palindromic nucleic acid (CRISPR)/Cas9, and/or a TAL-effector nuclease (TALEN). In some of any such embodiments, the disruption includes a deletion of at least a portion of at least one exon of the gene. In some of any such embodiments, the disruption includes a deletion, mutation, and/or insertion in the gene resulting in the presence of a premature stop codon in the gene; and/or the disruption includes a deletion, mutation, and/or insertion within a first or second exon of the gene. In some of any such embodiments, expression of PD-L1 in the T cell is reduced by at least 50, 60, 70, 80, 90, or 95% as compared to the expression in the T cell in the absence of the inhibitory nucleic acid molecule or gene disruption or in the absence of activation thereof.

In some of any such embodiments, a genetically engineered T cell contains a genetically engineered antigen receptor that specifically binds to an antigen; and a polynucleotide encoding a molecule that reduces or disrupts expression of PD-1 or PD-L1 in the cell, wherein expression or activity of the polynucleotide is conditional. In some of any such embodiments, expression is under the control of a conditional promoter or enhancer or transactivator. In some of any such embodiments, the conditional promoter or enhancer or transactivator is an inducible promoter, enhancer, or transactivator or a repressible promoter, enhancer, or transactivator. In some of any such embodiments, the molecule that reduces or disrupts expression of PD-1 or PD-L1 is or includes or encodes an antisense molecule, siRNA, shRNA, miRNA, a gene editing nuclease, zinc finger nuclease protein (ZFN), a TAL-effector nuclease (TALEN) or one or more components of a CRISPR-Cas9 combination that specifically binds to, recognizes, or hybridizes to the gene.

In some of any such embodiments, the promoter is selected from among an RNA pol I, pol II or pol III promoter. In some of any such embodiments, the promoter is selected from: a pol III promoter that is a U6 or H1 promoter; or a pol II promoter that is a CMV, SV40 early region or adenovirus major late promoter. In some of any such embodiments, the promoter is an inducible promoter. In some of any such embodiments, the promoter includes a Lac operator sequence, a tetracycline operator sequence, a galactose operator sequence or a doxycycline operator sequence, or is an analog thereof.

In some of any such embodiments, the promoter is a repressible promoter. In some of any such embodiments, the promoter includes a Lac repressible element or a tetracycline repressible element, or is an analog thereof.

In some of any such embodiments, the T cell is a CD4+ or CD8+ T cell. In some of any such embodiments, the genetically engineered antigen receptor is a functional non-T cell receptor.

In some of any such embodiments, the genetically engineered antigen receptor is a chimeric antigen receptor (CAR). In some of any such embodiments, the CAR contains an extracellular antigen-recognition domain that specifically binds to the antigen and an intracellular signaling domain including an ITAM. In some of any such embodiments, the intracellular signaling domain includes an intracellular domain of a CD3-zeta (CD3ζ) chain. In some of any such embodiments, the CAR further contains a costimulatory signaling region. In some of any such embodiments, the costimulatory signaling region contains a signaling domain of CD28 or 4-1BB. In some of any such embodiments, the costimulatory domain is CD28.

In some of any such embodiments, the cell is a human cell. In some of any such embodiments, the cell is an isolated cell.

In some embodiments, also provided is a nucleic acid molecule that contains a first nucleic acid, which is optionally a first expression cassette, encoding an antigen receptor (CAR) and a second nucleic acid, which is optionally a second expression cassette, encoding an inhibitory nucleic acid molecule against a gene encoding PD-1 or PD-L1 and/or a nucleic acid sequence that is complementary to a gene encoding PD-1 or PD-L1. In some of any such embodiments, the inhibitory nucleic acid molecule contains an RNA interfering agent. In some of any such embodiments, the inhibitory nucleic acid is or contains or encodes a small interfering RNA (siRNA), a microRNA-adapted shRNA, a short hairpin RNA (shRNA), a hairpin siRNA, a precursor microRNA (pre-miRNA) a pri-miRNA, or a microRNA (miRNA). In some of any such embodiments, the inhibitory nucleic acid contains a sequence complementary to a PD-1-encoding nucleic acid; in some of any such embodiments, it contains a sequence complementary to a PD-L1-encoding nucleic acid. In some of any such embodiments, the inhibitory nucleic acid molecule includes an antisense oligonucleotide complementary to a PD-1-encoding nucleic acid; in some of any such embodiments, the inhibitory nucleic acid molecule includes an antisense oligonucleotide complementary to ar PD-L1-encoding nucleic acid. In some embodiments, the second nucleic acid comprises a gRNA sequence comprising a targeting domain that is complementary with a target domain of the gene encoding PD-1 or PD-L1. In some such embodiments, the nucleic acid molecule can further comprise a third nucleic acid encoding a Cas9 molecule, which, in some cases comprises an enzymatically inactive Cas9 (eiCas9 or iCas9) or an eiCas9 fusion protein.

In some embodiments, each of the one or more nucleic acids can be separated by an element to permit translation of multiples genes from the same transcript. In some embodiments, the nucleic acid molecule is multicistronic, such as bicistronic. In some embodiments, the element is or comprises an Internal Ribosome Entry Site (IRES) or comprises a skip sequence such as a sequence encoding a self-cleaving 2A peptide (e.g. T2A, P2A, E2A or F2A).

In some of any such embodiments, the nucleic acid encodes an antigen receptor that is a functional non-T cell receptor. In some of any such embodiments, the genetically engineered antigen receptor is a chimeric antigen receptor (CAR). In some of any such embodiments, the CAR contains an extracellular antigen-recognition domain that specifically binds to the antigen and an intracellular signaling domain containing an ITAM. In some of any such embodiments, the intracellular signaling domain contains an intracellular domain of a CD3-zeta (CD3ζ) chain. In some of any such embodiments, the CAR further includes a costimulatory signaling region. In some of any such embodiments, the costimulatory signaling region includes a signaling domain of CD28 or 4-1BB. In some of any such embodiments, the costimulatory domain is CD28.

In some of any such embodiments, the first and second nucleic acids, optionally the first and second expression cassettes, are operably linked to the same or different promoters. In some of any such embodiments, the first nucleic acid, optionally first expression cassette, is operably linked to an inducible promoter or a repressible promoter and the second nucleic acid, optionally second expression cassette, is operably linked to a constitutive promoter.

In some of any such embodiments, the nucleic acid is isolated. In embodiments, also provided is a vector that contains the nucleic acid of some or any embodiments. In some of any such embodiments, the vector is a plasmid, lentiviral vector, retroviral vector, adenoviral vector, or adeno-associated viral vector. In some of any such embodiments, the vector is integrase defective.

In some embodiments, also provided is a T cell that contains the nucleic acid molecule or vector. In some of any such embodiments, the T cell is a CD4+ or CD8+ T cell. In some of any such embodiments, the T cell is a human cell. In some of any such embodiments, the T cell is isolated.

In some embodiments, also provided is a pharmaceutical composition that contains the cell of some of any of the embodiments described herein and a pharmaceutically acceptable carrier.

In some embodiments, also provided is a method of producing a genetically engineered T cell, that includes the steps of: (a) introducing a genetically engineered (recombinant) antigen receptor that specifically binds to an antigen into a population of cells including T cells, such as by introducing nucleic acid molecule encoding the antigen receptor into the cell; and (b) introducing into the population of cells an agent capable of leading to a reduction of expression of PD-L1 and/or inhibiting upregulation of PD-L1 in T cells in the population upon incubation under one or more conditions, as compared to PD-L1 expression and/or upregulation in T cells in a corresponding population of cells not introduced with the agent upon incubation under the one or more conditions, wherein steps (a) and (b) are carried out simultaneously or sequentially in any order, thereby introducing the genetically engineered antigen receptor and the agent into a T cell in the population.

In some of any such embodiments, a method of regulating expression of PD-L1 in a genetically engineered T cell includes introducing into a T cell an agent capable of leading to a reduction of expression of PD-L1 and/or inhibiting upregulation of PD-L1 in the cell upon incubation under one or more conditions, as compared to expression or upregulation of PD-L1 in a corresponding T cell not introduced with the agent upon incubation under the one or more conditions, said T cell containing a genetically engineered antigen receptor that specifically binds to an antigen. In some of any such embodiments, incubation under conditions including the presence of antigen induces expression or upregulation of PD-L1 in the corresponding population containing T cells not introduced with the agent.

In some of any such embodiments, the incubation in the presence of antigen includes incubating the cells in vitro with the antigen. In some of any such embodiments, the incubation in the presence of antigen is for 2 hours to 48 hours, 6 hours to 30 hours or 12 hours to 24 hours, each inclusive, or is for less than 48 hours, less than 36 hours or less than 24 hours.

In some of any such embodiments, the incubation includes administration of the cells to a subject under conditions whereby the engineered antigen receptor specifically binds to the antigen for at least a portion of the incubation. In some of any such embodiments, the incubation induces expression or upregulation within a period of 24 hours, 2 days, 3 days, 4 days, 5 days, 6 days, 7 days, 8 days, 9 days or 10 days following administration of cells to the subject. In some of any such embodiments, the reduction in expression or inhibition of upregulation of PD-L1 is by at least or at least about 30%, 40%, 50%, 60%, 70%, 80%, 90%, 95% or more.

In some of any such embodiments, the method is performed ex vivo. In some of any such embodiments, the introducing of the agent is carried out by introducing a nucleic acid containing a sequence encoding the agent. In some embodiments, the introducing of the agent comprises introducing at least one complex of a Cas9 molecule, such as an enzymatically inactive Cas9 (e.g. eiCas9) or fusion protein thereof, and a gRNA having a targeting domain that is complementary with a target domain of the gene encoding PD-L1. In some of any such embodiments, the introducing includes inducing transient expression of the agent in the T cell to effect temporary reduction or disruption of expression of PD-L1 in the cell, and/or wherein the reduction or disruption is not permanent.

In some of any such embodiments, expression or activity of the agent is conditional. In some of any such embodiments, the expression is under the control of a conditional promoter or enhancer or transactivator. In some of any such embodiments, the conditional promoter or enhancer or transactivator is an inducible promoter, enhancer or transactivator or a repressible promoter, enhancer or transactivator. In some of any such embodiments, the promoter is selected from an RNA pol I, pol II or pol III promoter. In some of any such embodiments, the promoter is selected from: a pol III promoter that is a U6 or an H1 promoter; or a pol II promoter that is a CMV, a SV40 early region or an adenovirus major late promoter.

In some of any such embodiments, the promoter is an inducible promoter. In some of any such embodiments, the promoter includes a Lac operator sequence, a tetracycline operator sequence, a galactose operator sequence or a doxycycline operator sequence. In some of any such embodiments, the promoter is a repressible promoter. In some of any such embodiments, the promoter includes a Lac repressible element or a tetracycline repressible element.

In some of any such embodiments, the agent is stably expressed in the T cell to effect continued reduction or disruption of expression of PD-L1 in the cell. In some of any such embodiments, the agent is a nucleic acid molecule that is contained in a viral vector. In some of any such embodiments, the viral vector is an adenovirus, lentivirus, retrovirus, herpesvirus or adeno-associated virus vector. In some of any such embodiments, the agent is an inhibitory nucleic acid molecule that reduces expression of PD-L1 in the cell.

In some of any such embodiments, the inhibitory nucleic acid molecule includes an RNA interfering agent. In some of any such embodiments, the inhibitory nucleic acid is or includes or encodes a small interfering RNA (siRNA), a microRNA-adapted shRNA, a short hairpin RNA (shRNA), a hairpin siRNA, a precursor microRNA (pre-miRNA), a pri-miRNA, or a microRNA (miRNA). In some of any such embodiments, the inhibitory nucleic acid molecule contains a sequence complementary to a PD-L1-encoding nucleic acid. In some of any such embodiments, the inhibitory nucleic acid molecule contains an antisense oligonucleotide complementary to a PD-L1-encoding nucleic acid. In some embodiments, the nucleic acid comprises a gRNA sequence comprising a targeting domain that is complementary with a target domain of the gene encoding PD-L1. In some such embodiments, the nucleic acid molecule can further comprise a third nucleic acid encoding a Cas9 molecule, which, in some cases comprises an enzymatically inactive Cas9 (eiCas9 or iCas9) or an eiCas9 fusion protein.

In some of any such embodiments, the effecting reduction and/or inhibiting upregulation in the provided methods includes disrupting a gene encoding PD-L1. In some of any such embodiments, the disruption includes disrupting the gene at the DNA level and/or the disruption is not reversible; and/or the disruption is not transient.

In some of any such embodiments, the disruption includes introducing an agent that is a DNA binding protein or DNA-binding nucleic acid that specifically binds to or hybridizes to the gene. In some of any such embodiments, the disruption includes introducing: (i) a fusion protein containing a DNA-targeting protein and a nuclease or (ii) an RNA-guided nuclease. In some of any such embodiments, the DNA-targeting protein or RNA-guided nuclease contains a zinc finger protein (ZFP), a TAL protein, or a Cas protein (e.g. Cas9) guided by a clustered regularly interspaced short palindromic nucleic acid (CRISPR) specific for the gene (CRISPR/Cas). In some of any such embodiments, the disruption includes introducing a zinc finger nuclease (ZFN), a TAL-effector nuclease (TALEN), or and a CRISPR-Cas9 combination that specifically binds to, recognizes, or hybridizes to the gene. In some of any such embodiments, the introducing is carried out by introducing a nucleic acid containing a sequence encoding the DNA-binding protein, DNA-binding nucleic acid, and/or complex including the DNA-binding protein or DNA-binding nucleic acid.

In some of any such embodiments, the nucleic acid is in a viral vector. In some of any such embodiments, the specific binding to the gene is within an exon of the gene and/or is within a portion of the gene encoding an N-terminus of the target antigen. In some of any such embodiments, the introduction thereby effects a frameshift mutation in the gene and/or an insertion of an early stop codon within the coding region of the gene.

In some of any such embodiments, the method further includes introducing into the cell an agent capable of leading to a reduction of expression of PD-1 and/or inhibiting upregulation of PD-1 in the cell upon incubation under the one or more conditions compared to PD-1 expression or upregulation in a corresponding cell not introduced with the agent upon incubation under the one or more conditions, wherein the reduction of expression and/or inhibition of upregulation is temporary or transient. In some of any such embodiments, the agent is inducibly expressed or repressed in the cell to effect conditional reduction or disruption of expression of PD-1 in the cell.

In some embodiments, also provided is a method of producing a genetically engineered T cell that includes (a) introducing a genetically engineered antigen receptor that specifically binds to an antigen into a population of cells containing T cells, such as by introducing nucleic acid molecule encoding the antigen receptor into the cells; and (b) introducing into the population of cells an agent capable of transient reduction of expression of PD-1 and/or a transient inhibition of upregulation of PD-1 in T cells in the population upon incubation under one or more conditions, as compared to PD-1 expression and/or upregulation in T cells in a corresponding population of cells not introduced with the agent upon incubation under the one or more conditions, wherein steps (a) and (b) are carried out simultaneously or sequentially in any order, thereby introducing the genetically engineered antigen receptor and the agent into a T cell in the population.

In some of any such embodiments, a method of regulating expression of PD-1 in a genetically engineered T cell includes introducing into a T cell an agent capable of transient reduction of expression of PD-1 and/or a transient inhibition of upregulation of PD-1 in the cell upon incubation under one or more conditions, as compared to expression or upregulation of PD-1 in a corresponding T cell not introduced with the agent upon incubation under the one or more conditions, said T cell contains an antigen receptor that specifically binds to an antigen.

In some of any such embodiments, transient reduction includes reversible reduction in expression of PD-1 in the cell. In some of any such embodiments, incubation under conditions including the presence of antigen induces expression or upregulation of PD-1 in the corresponding population containing T cells not introduced with the agent. In some of any such embodiments, the incubation in the presence of antigen includes incubating the cells in vitro with the antigen. In some of any such embodiments, the incubation in the presence of antigen is for 2 hours to 48 hours, 6 hours to 30 hours or 12 hours to 24 hours, each inclusive, or is for less than 48 hours, less than 36 hours or less than 24 hours. In some of any such embodiments, the incubation includes administration of the cells to a subject under conditions whereby the engineered antigen receptor specifically binds to the antigen for at least a portion of the incubation. In some of any such embodiments, the incubation induces expression or upregulation within a period of 24 hours, 2 days, 3 days, 4 days, 5 days, 6 days, 7 days, 8 days, 9 days or 10 days following administration of cells to the subject. In some of any such embodiments, the reduction in expression or inhibition of upregulation of PD-1 is by at least or at least about 30%, 40%, 50%, 60%, 70%, 80%, 90%, 95% or more. In some of any such embodiments, the method is performed ex vivo.

In some of any such embodiments, the introducing the agent is carried out by introducing into the cell a nucleic acid containing a sequence encoding the agent, e.g. an inhibitory nucleic acid molecule against PD-1 and/or a nucleic acid sequence that is complementary to or binds to a gene encoding PD-1. In some embodiments, the agent comprises a gRNA having a targeting domain that is complementary with a target domain of the gene encoding PD-1 in combination with a Cas9 molecule, such as an enzymatically inactive Cas9 (e.g. eiCas9) or a eiCas9 fusion protein for reducing or repressing gene expression. In some embodiments, the agent comprises nucleic acid molecules encoding the at least one gRNA and/or the Cas9 molecule. In some embodiments, the agent comprises at least one complex of the Cas9 molecule and a gRNA having a targeting domain that is complementary with a target domain of the PD-1 gene.

In some of any such embodiments, the agent is transiently expressed in the cell to effect temporary reduction or disruption of expression of PD-1 in the T cell. In some of any such embodiments, the expression or activity of the agent is conditional. In some of any such embodiments, the expression is under the control of a conditional promoter or enhancer or transactivator. In some of any such embodiments, the conditional promoter or enhancer or transactivator is an inducible promoter, enhancer or transactivator is a repressible promoter, enhancer or transactivator.

In some of any such embodiments, the promoter is selected from an RNA pol I, pol II or pol III promoter. In some of any such embodiments, the promoter is selected from: a pol III promoter that is a U6 or an H1 promoter; or a pol II promoter that is a CMV, a SV40 early region or an adenovirus major late promoter. In some of any such embodiments, the promoter is an inducible promoter. In some of any such embodiments, the promoter includes a Lac operator sequence, a tetracycline operator sequence, a galactose operator sequence or a doxycycline operator sequence. In some of any such embodiments, the promoter is a repressible promoter. In some of any such embodiments, the promoter includes a Lac repressible element or a tetracycline repressible element.

In some of any such embodiments, the agent is an inhibitory nucleic acid molecule that reduces expression of PD-1 in the cell. In some of any such embodiments, the inhibitory nucleic acid molecule includes an RNA interfering agent. In some of any such embodiments, the inhibitory nucleic acid is or includes or encodes a small interfering RNA (siRNA), a microRNA-adapted shRNA, a short hairpin RNA (shRNA), a hairpin siRNA, a precursor microRNA (pre-miRNA) or a microRNA (miRNA). In some of any such embodiments, the inhibitory nucleic acid molecule includes a sequence complementary to a PD-L1-encoding nucleic acid. In some of any such embodiments, the inhibitory nucleic acid molecule contains an antisense oligonucleotide complementary to a PD-L1-encoding nucleic acid.

In some of any such embodiments of the provided methods, the T cell is a CD4+ or CD8+ T cell. In some of any such embodiments, the genetically engineered antigen receptor is a functional non-T cell receptor. In some of any such embodiments, the genetically engineered antigen receptor is a chimeric antigen receptor (CAR). In some of any such embodiments, the CAR includes an extracellular antigen-recognition domain that specifically binds to the antigen and an intracellular signaling domain including an ITAM. In some of any such embodiments, the intracellular signaling domain includes an intracellular domain of a CD3-zeta (CD3ζ) chain. In some of any such embodiments, the CAR further includes a costimulatory signaling region. In some of any such embodiments, the costimulatory signaling region includes a signaling domain of CD28 or 4-1BB. In some of any such embodiments, the costimulatory domain is CD28.

In some of any such embodiments, the steps of introducing the genetically engineered (recombinant) antigen receptor and the agent are performed simultaneously, said steps including introducing a nucleic acid molecule containing a first nucleic acid, which is optionally a first expression cassette, encoding the antigen receptor and a second nucleic acid, which is optionally a second expression cassette, encoding the agent to effect reduction of expression of PD-1 or PD-L1.

In some of any such embodiments, any of the provided methods further including introducing into the population of cells a second genetically engineered antigen receptor that specifically binds to the same or a different antigen, said second antigen receptor containing a co-stimulatory molecule other than CD28.

In some embodiments, also provided is a method of producing a genetically engineered T cell includes (a) introducing a first genetically engineered antigen receptor that specifically binds to a first antigen into a population of cells containing T cells, said first antigen receptor including a CD28 co-stimulatory molecule, wherein the introducing of the first genetically engineered antigen receptor can be by introducing a nucleic acid molecule encoding the first antigen receptor into the cell; (b) introducing into the population of cells containing T cells a second genetically engineered antigen receptor that specifically binds to the same or different antigen, such as by introducing a nucleic acid molecule encoding the second antigen receptor; and (c) introducing into the population of cells including T cells an agent capable of leading to a reduction of expression of PD-1 or PD-L1 and/or inhibiting upregulation of PD-1 or PD-L1 in T cells in the population upon incubation under one or more conditions, as compared to PD-1 and/or PD-L1 expression or upregulation in T cells in a corresponding population of cells not introduced with the agent upon incubation under the one or more conditions, thereby introducing the first antigen receptor, the second antigen receptor and the agent into a T cell in the population.

In some of any such embodiments, incubation under conditions including the presence of antigen induces expression or upregulation of PD-1 and/or PD-L1 in the corresponding population containing T cells not introduced with the agent.

In some of any such embodiments, the incubation in the presence of antigen includes incubating the cells in vitro with the antigen. In some of any such embodiments, the incubation in the presence of antigen is for 2 hours to 48 hours, 6 hours to 30 hours or 12 hours to 24 hours, each inclusive, or is for less than 48 hours, less than 36 hours or less than 24 hours. In some of any such embodiments, the incubation includes administration of the cells to a subject under conditions whereby the engineered antigen receptor specifically binds to the antigen for at least a portion of the incubation. In some of any such embodiments, the incubation induces expression or upregulation within a period of 24 hours, 2 days, 3 days, 4 days, 5 days, 6 days, 7 days, 8 days, 9 days or 10 days following administration of cells to the subject. In some of any such embodiments, expression or upregulation of PD-1 and/or PD-L1 in the cells in inhibited or reduced by at least or at least about 30%, 40%, 50%, 60%, 70%, 80%, 90%, 95% or more compared to an engineered cell produced by the method in the absence of introducing the agent.

In some of any such embodiments, the first and second genetically engineered antigen receptors bind the same antigen. In some of any such embodiments, the second antigen receptor includes a co-stimulatory molecule other than CD28. In some of any such embodiments, the costimulatory molecule other than CD28 is 4-1BB. In some of any such embodiments, the agent effects reduction of expression and/or inhibition of upregulation of PD-L1.

In some of any such embodiments, introducing the first antigen receptor, second antigen receptor and/or agent are performed simultaneously, said steps including introducing a nucleic acid molecule containing a first nucleic acid, which is optionally a first expression cassette, encoding the first antigen receptor, a second nucleic acid, which is optionally a second expression cassette, encoding the second antigen receptor and a third nucleic acid, which is optionally a third expression cassette, encoding the agent to effect reduction of expression of PD-1 or PD-L1. In some of any such embodiments, the first, second and/or third nucleic acids, optionally the first, second and/or third expression cassettes, are operably linked to the same or different promoters. In some of any such embodiments, the first and/or second nucleic acid, optionally first and/or second expression cassette, is operably linked to an inducible promoter or a repressible promoter and the third nucleic acid, optionally third expression cassette, is operably linked to a constitutive promoter.

In some of any such embodiments, the method involves introducing such molecules or agents into a human cell.

In some embodiments, provided is a method of producing a genetically engineered T cell that includes (a) obtaining a population of primary cells containing T cells; (b) enriching for cells in the population that do not express a target antigen; and (c) introducing into the population of cells a genetically engineered antigen receptor that specifically binds to the target antigen; thereby producing a genetically engineered T cell.

In some of any such embodiments, the method further including culturing and/or incubating the cells under stimulating conditions to effect proliferation of the cells, wherein the proliferation and/or expansion of cells is greater than in cells produced in the method but in the absence of enriching for cells that do not express the target antigen. In some of any such embodiments, proliferation and/or expansion of cells is at least or at least about 1.5-fold, 2-fold, 3-fold, 4-fold, 5-fold, 6-fold, 7-fold, 8-fold, 9-fold, 10-fold or greater. In some of any such embodiments, enriching for cells that do not express a target antigen includes negative selection to deplete cells expressing the target antigen or disruption of the gene encoding the target antigen in cells in the population.

In some of any such embodiments, the stimulating condition includes an agent capable of activating one or more intracellular signaling domains of one or more components of a TCR complex.

In some embodiments, provided is a cell is produced by any of the methods described herein. In some embodiments, provided is a pharmaceutical composition that includes the cell and a pharmaceutically acceptable carrier.

In some embodiments, provided is a method of treatment includes administering to a subject having a disease or condition the cell or the pharmaceutical composition. In some of any such embodiments, the cells are administered in a dosage regime involving (a) administering to the subject a first dose of cells expressing a chimeric antigen receptor (CAR); and (b) administering to the subject a consecutive dose of CAR-expressing cells, said consecutive dose being administered to the subject at a time when expression of PD-L1 is induced or upregulated on the surface of the CAR-expressing cells administered to the subject in (a) and/or said consecutive dose being administered to the subject at least 5 days after initiation of the administration in (a).

In some embodiments, provided is a method that includes (a) administering to the subject a first dose of cells expressing a chimeric antigen receptor (CAR); and (b) administering to the subject a consecutive dose of CAR-expressing cells said consecutive dose being administered to the subject at a time when expression of PD-L1 is induced or upregulated on the surface of the CAR-expressing cells administered to the subject in (a) and/or said consecutive dose being administered to the subject at least 5 days after initiation of the administration in (a).

In some of any such embodiments, the method includes a consecutive dose of cells that is administered at least or more than about 5 days after and less than about 12 days after initiation of said administration in (a). In some of any such embodiments, the number of cells administered in the first and/or second dose is between about 0.5×10⁶ cells/kg body weight of the subject and 4×10⁶ cells/kg, between about 0.75×10⁶ cells/kg and 3.0×10⁶ cells/kg or between about 1×10⁶ cells/kg and 2×10⁶ cells/kg, each inclusive.

In some of any such embodiments, the genetically engineered antigen receptor specifically binds to an antigen associated with the disease or condition. In some of any such embodiments, the disease or condition is a cancer. In some of any such embodiments, the disease or condition is a leukemia or lymphoma. In some of any such embodiments, the disease or condition is acute lymphoblastic leukemia. In some of any such embodiments, the disease or condition is a non-Hodgkin lymphoma (NHL).

### BRIEF DESCRIPTION OF THE DRAWINGS

FIG. 1A: depicts surface expression, as detected by flow cytometry, of PD-1, PD-L1, and PD-L2 on a population of T cells gated for positive surface expression of CD4 and an anti-CD19 chimeric antigen receptor (CAR) (gating strategy shown in top panel), following incubation for 24 hours under various conditions (media, K562-tCD19, K562-tROR1, aCD3/aCD28), as described in Example 1.

FIG. 1B: depicts surface expression, as detected by flow cytometry, of PD-1, PD-L1, and PD-L2 on a population of T cells gated for positive surface expression of CD4 and negative surface expression of an anti-CD19 chimeric antigen receptor (CAR) (gating strategy shown in top panel), following incubation for 24 hours under various conditions (media, K562-tCD19, K562-tROR1, aCD3/aCD28), as described in Example 1.

FIG. 2A: depicts surface expression, as detected by flow cytometry, of PD-1, PD-L1, and PD-L2 on a population of T cells gated for positive surface expression of CD8 and an anti-CD19 chimeric antigen receptor (CAR) (gating strategy shown in top panel), following incubation for 24 hours under various conditions (media, K562-tCD19, K562-tROR1, aCD3/aCD28), as described in Example 1.

FIG. 2B: depicts surface expression, as detected by flow cytometry, of PD-1, PD-L1, and PD-L2 on a population of T cells gated for positive surface expression of CD8 and negative surface expression for an anti-CD19 chimeric antigen receptor (CAR) (gating strategy shown in top panel), following incubation for 24 hours under various conditions (media, K562-tCD19, K562-tROR1, aCD3/aCD28), as described in Example 1.

### DETAILED DESCRIPTION

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Compositions and Methods for Reducing Immunosuppression and Inhibitory Interactions in Adoptive Cell Therapy

Provided are methods, cells (such as T cells expressing genetically engineered receptors such as CARs), compositions, and nucleic acids, for use in adoptive cell therapy, e.g., adoptive immunotherapy. In some aspects, the provided embodiments enhance the efficacy or longevity of adoptive cell therapy, for example, in the context of solid tumors or tumor microenvironments delivering immunoinhibitory signals. The methods generally involve disrupting the effects of certain T cell inhibitory pathways or signals, which might otherwise impair certain desirable effector functions in the context of cancer therapy. Thus, provided are compositions and methods that enhance T cell function in adoptive cell therapy, including those offering improved efficacy, such as by increasing activity and potency of administered genetically engineered (e.g., CAR+) cells, while maintaining persistence or exposure to the transferred cells over time. In some embodiments, the genetically engineered cells, such as CAR-expressing T cells, exhibit increased expansion and/or persistence when administered in vivo to a subject, as compared to certain available methods.

The provided methods, cells and compositions regulate and/or modulate inhibitory interactions, such as reduce or inhibit inhibitory interactions, from occurring in cells engineered with an antigen receptor, such as in cells containing a chimeric antigen receptor (CAR). In some embodiments, the provided embodiments regulate, such as reduce or inhibit, inhibitory interactions between programmed death-1 (PD-1) and its ligand PD-L1 in genetically engineered T cells, such as CAR-expressing cells, that can result from co-expression of these molecules on T cells. Thus, in some embodiments, the provided embodiments are advantageous by way of reducing or eliminating loss of function that can occur in genetically engineered T cells, such as CAR-expressing cells, by actions of inhibitory molecules on the cells as compared with other methods and products.

In some embodiments, the compositions and methods involve the disruption of signals delivered via the immune checkpoint molecule PD-1, such as by disrupting expression of one or more PD-1 ligand(s) in adoptively transferred, e.g., CAR+, T cells. Tumor cells and/or cells in the tumor microenvironment often upregulate ligands for PD-1 (such as PD-L1 and PD-L2), which in turn leads to ligation of PD-1 on tumor-specific T cells expressing PD-1, delivering an inhibitory signal. PD-1 also often is upregulated on T cells in the tumor microenvironment, e.g., on tumor-infiltrating T cells, which can occur following signal through the antigen receptor or certain other activating signals.

The interaction between T cells induced to express PD-1 and PD-L1 or PD-L2-expressing cells in the tumor microenvironment can impair anti-tumor immunity and/or the function or efficacy of adoptively transferred T cells. For example, signaling through the PD-1 molecule on T cells can promote exhaustion or anergy and/or inhibit proliferation or effector function(s). Certain methods have been aimed at blocking PD-1 signaling or disrupting PD-1 expression in T cells, including in the context of cancer therapy. Such blockade or disruption may be through the administration of blocking antibodies, small molecules, or inhibitory peptides, or through the knockout or reduction of expression of PD-1 in T cells, e.g., in adoptively transferred T cells. The disruption of PD-1 in transferred T cells, however, may not be entirely satisfactory.

Among the provided cells, compositions, and uses are those with certain advantages compared to other approaches targeting the PD-1 signal to promote cancer therapy. For example, provided are cells, methods and compositions that inhibit detrimental effects of an inhibitory PD-1 signal in tumor-targeting T cells, without introducing certain negative impacts that can result from or be associated with certain PD-1 targeting approaches.

Whereas PD-1 expression and signaling can reduce certain effector functions and expansion of T cells, it also is associated with T cell longevity, differentiation and persistence of memory T cells (e.g., long-lived and/or central memory T cells) over time. For example, PD-1 signals have been shown to induce bioenergetics properties of long-lived cells. Disruption (e.g., knockdown or knockout) of PD-1 in anti-tumor T cells can improve efficacy in the near term, by promoting cell expansion, secretion of cytokines, and other effector functions, particularly in the context of a tumor microenvironment in which ligand(s) for PD-1 are present or upregulated. Yet despite these enhancements, disrupting PD-1 in adoptively transferred cells may reduce the number or percentage of these cells with a memory or central memory phenotype over time. Disruption of PD-1 in T cells can lead to a reduction in long-lived memory T cell compartment and/or central memory compartment of PD-1-deficient T cell populations, such as central memory compartment (e.g., long-lived memory CD8+ T cells and/or CD8+ central memory T cells) and/or reduces the potential of these cells for survival long-term.

Thus, whereas disruption of PD-1 (e.g., by knockdown or knockout) in genetically engineered T cells can promote their effector function, it may not be optimal long-term due to impairment of the ability of the engineered cells to persist long-term in the memory compartment and/or to differentiate into memory cell subsets that can be important for long-term exposure and anti-tumor efficacy. Thus, while blockade of PD-1 function in adoptively transferred T cells is attractive in some respects as a mechanism for promoting efficacy in the face of inhibitory signals of the tumor microenvironment, it may not be the optimal choice in the long run. Provided are methods and compositions for reducing the negative effects of this pathway on tumor-targeting T cells without certain negative consequences that can compromise efficacy long-term.

In some aspects, the provided compositions and methods are based in part on the observation of that PD-L1—a ligand for the T cell checkpoint molecule PD-1, which is ordinarily expressed on non-T cells and responsible for delivering the negative signal to T cells through PD-L1—can be rapidly (e.g., within 24 hours) upregulated on the surface of CAR-expressing T cells cultured in the presence of cells expressing the antigen for which the CAR is specific. In studies presented herein, whereas both PD-L1 and PD-1 were rapidly upregulated in response to such signals, neither molecule was upregulated substantially beyond levels observed in control samples within this timeframe in response to conditions that mimic signals through the canonical T cell receptor complex and associated costimulatory signals (anti-CD3/anti-CD28 stimulation). Thus, in some embodiments, the provided embodiments are based on the observations herein that incubation of CAR-expressing T cells in the presence of antigen specific to the CAR can rapidly upregulate PD-1 and PD-L1 expression in the cells. Preliminary results indicate that, in some aspects, this upregulation occurs quickly and within 24 hours following incubation with antigen in vitro. In contrast, upregulation of either PD-1 or PD-L1 did not occur in the cells following stimulation under conditions designed to mimic signal through the canonical T cell antigen receptor complex and associated costimulatory receptors (such as anti-CD3/anti-CD28 antibodies) during the same time period.

Thus, such cells upon encounter with a tumor expressing the target antigen, may upregulate not only PD-1 but also PD-L1, leading to negative self-regulation or regulation by transferred T cells of other transferred or other T cells within the tumor environment. PD-1 and/or PD-L1 can also be upregulated in certain contexts, e.g., within longer timeframes, in response to canonical signals through the TCR complex.

In other words, observations herein indicate that, in some cases, stimulation through the engineered and artificial receptor, via its antigen, can result in upregulation of co-expressed inhibitory molecule pairs, such as PD-1 and PD-L1, and/or such inhibitory pairs, one on each of two different T cells, which may contribute to self-downregulation or inhibition (or inhibition by T cells in trans) of T cell activity, expansion, or effector function, in the presence of or following antigen encounter. In some aspects, this regulation or negative impact may occur in CAR-expressing cells at a time that is earlier than, or to a degree that is greater than, that which may occur in some aspects when T cells are stimulated via its natural antigen receptor complex.

In some cases, such events may contribute to genetically engineered (e.g., CAR+) T cells acquiring an exhausted phenotype after antigen-antigen receptor binding, or when present in proximity with other cells that have encountered antigen and upregulated PD-L1, which in turn can lead to reduced functionality. Exhaustion of T cells may lead to a progressive loss of T cell functions and/or in depletion of the cells (Yi et al. (2010) Immunology, 129:474-481). T cell exhaustion and/or the lack of T cell persistence is a barrier to the efficacy and therapeutic outcomes of adoptive cell therapy; clinical trials have revealed a correlation between greater and/or longer degree of exposure to the antigen receptor (e.g. CAR)-expressing cells and treatment outcomes.

In some embodiments, the methods and compositions provide for the deletion, knockout, disruption, or reduction in expression of PD-L1 in T cells to be adoptively transferred (such as cells engineered to express a CAR or transgenic TCR), and in some aspects without also disrupting or otherwise impairing expression or function of PD-1 in such cells to be adoptively transferred. Accordingly, the transferred cells would be capable of upregulating PD-1 and receiving signals through cells other than other transferred T cells, which may improve longevity of transferred cells including in the memory compartment. Thus, the provided methods in some aspects can reduce the negative effects of this self-regulation, while avoiding long-term impairment of long-lived memory CAR+ T cells which may otherwise occur in the context of PD-1 knockdown or knockout in these cells. In some embodiments, the deletion, knockout, disruption, reduction of expression, disruption of expression, inhibition of upregulation and/or inhibition of function of genes or other nucleic acids or biomolecules encoding PD-1 or PD-L1, or PD-1 or PD-L1 molecules, is effected at the genomic level (e.g., knockout, gene-editing, knockin, genomic deletion), transcriptional level (e.g., transcriptional repression, transcriptional knockdown), post-transcriptional level, translational level, post-translational level, level of cellular transport, level of surface expression or level of functional activity.

Also provided are methods in which one or more consecutive doses of engineered cells are administered. As described herein, upon upregulation of PD-1 or PD-L1 in cells upon encounter with the antigen recognized by the engineered receptor, e.g., CAR, the cells of a first dose may become exhausted and/or less efficacious. By providing fresh cells at a time when this has occurred or has been observed to occur or at a time that such event typically occurs in the subject or disease state, the provided methods provide a fresh dose of cells that are not exhausted or anergized and are not poised to deliver a negative signal via a PD-L1 molecule, increasing exposure.

Also provided are methods in which PD-1 and/or PD-L1 expression is transiently and/or inducibly disrupted in the adoptively transferred cells. For example, in some embodiments, the methods involve the administration of an agent that disrupts or reduces expression of PD-1 or PD-L1, which disruption is not permanent, such that cells upon transfer are permitted to encounter antigen, expand, and exert effector functions such as cell killing or cytotoxicity, without or with reduced risk of inhibition or exhaustion by way of PD-1/PD-L1 upregulation. Because such downregulation is transient, it can be advantageous in not being associated with certain long-term negative impacts such as impaired long-lived memory differentiation or persistence. After the transient disruption is ceased, cells may upregulate and receive signals through PD-1, promoting long-lived memory generation and persistence. In some embodiments, transient disruption is provided by the downregulation of expression, e.g., by administering to the cells an agent, such as one or more nucleic acids and/or polypeptides or combinations or complexes thereof, that effect targeted disrupted gene expression for a limited period of time following administration. Transient expression may be effected by genetic engineering techniques placing a gene under the control of a promoter or enhancer or other control system that permits induction or reduction of its expression following delivery of another signal, such as following administration of a compound or other agent that activates or blocks such control. In some embodiments, the reduction in expression is inducible, such that the cells are permitted to exert their effects in the absence of any regulation of PD-1 or PD-L1, but upon administration of another agent, such as when persistence of transferred cells is observed to be declining or have declined, PD-1 and/or PD-L1 expression may be disrupted in the cells, which may be transient or permanent.

Also provided are methods aimed at avoiding detrimental or impairing effects upon upregulation of one or both of a checkpoint molecule and ligand (e.g., PD-1/PD-L1) in ex vivo cultures used to prepare and engineer cells for adoptive cell therapy. In embodiments described herein, cells are incubated under conditions that do not promote such upregulation, such as by stimulation using agents other than incubation with antigen that is specifically bound by the CAR expressed by the cells. Such agents may include those designed to mimic a TCR/coreceptor signal, such as anti-CD3/anti-CD28 antibodies and/or cytokines. In some embodiments, the culture conditions do not include cytokines or other agents that promote PD-1 or PD-L1 expression and/or include cytokines that promote cell longevity or other desired features.

In some embodiments, the upregulation and/or expression of either one or both of a costimulatory inhibitory receptor or its ligand can negatively control T cell activation and T cell function. PD-1 is an immune inhibitory receptor that belongs to the B7:CD28 costimulatory molecular family and reacts with its ligands PD-L1 and PD-L2 to inhibit T cell function. Exemplary PD-1 amino acid and encoding nucleic acid sequences are set forth in SEQ ID NO:9 and 10, respectively. In some embodiments, the PD-1-encoding nucleotide is a PDCD1 gene. PD-L1 is generally primarily reported to be expressed on antigen presenting cells and/or cancer cells, where it interacts with T-cell-expressed PD-1, e.g., to inhibit the activation of the T cell. Exemplary PD-L1 amino acid and encoding nucleic acid sequences are set forth in SEQ ID NO: 7 and 8, respectively; see also GenBank Acc. No. AF233516. In some embodiments, the PD-L1-encoding nucleic acid is a CD274 gene. In some cases, PD-L1 also has been reported to be expressed on T cells. In some cases, interaction of PD-1 and PD-L1 suppresses activity of cytotoxic T cells and, in some aspects, can inhibit tumor immunity to provide an immune escape for tumor cells. In some embodiments, expression of PD-1 and PD-L1 on T cells and/or in the tumor microenvironment can reduce the potency and efficacy of adoptive T cell therapy.

Thus, in some embodiments, the provided cells include those in which certain genes have been reduced or disrupted, including genes that encode immune inhibitory molecules, such as one or both of PD-1 or PD-L1. In some embodiments, the step of reducing, suppressing or disrupting the expression of one or more inhibitory molecules, such as one or more of PD-1 and/or PD-L1, is performed ex vivo. In some aspects, methods of producing or generating such genetically engineered T cells include introducing into a population of cells containing T cells one or more nucleic acid encoding a genetically engineered antigen receptor (e.g. CAR) and one or more nucleic acid molecules encoding an agent or agents that reduce or disrupt, or that is/are capable of reducing or disrupting, a gene or genes that encode immune inhibitory molecule, such as one or both of PD-1 or PD-L1, i.e. an inhibitory nucleic acid molecule.

As used herein, the term “introducing” encompasses a variety of methods of introducing DNA into a cell, either in vitro or in vivo, such methods including transformation, transduction, transfection, and infection. Vectors are useful for introducing DNA encoding molecules into cells. Possible vectors include plasmid vectors and viral vectors. Viral vectors include retroviral vectors, lentiviral vectors, or other vectors such as adenoviral vectors or adeno-associated vectors.

The population of cells containing T cells can be cells that have been obtained from a subject, such as obtained from a peripheral blood mononuclear cells (PBMC) sample, an unfractionated T cell sample, a lymphocyte sample, a white blood cell sample, an apheresis product, or a leukapheresis product. In some embodiments, T cells can be separated or selected to enrich T cells in the population using positive or negative selection and enrichment methods. In some embodiments, the population contains CD4+, CD8+ or CD4+ and CD8+ T cells. In some embodiments, the step of introducing the nucleic acid encoding a genetically engineered antigen receptor and the step of introducing the agent can occur simultaneously or sequentially in any order. In some embodiments, subsequent to introduction of the genetically engineered antigen receptor (e.g. CAR) and one or more agents, the cells are cultured or incubated under conditions to stimulate expansion and/or proliferation of cells.

In some embodiments, the provided T cells, such as cells produced by the provided methods, exhibit a reduction of expression of one or more inhibitory molecules (e.g. PD-1 or PD-L1) and/or an inhibition of upregulation of one or more inhibitory molecules (e.g. PD-1 or PD-L1) when the T cells are otherwise incubated under conditions that may or are likely to lead to expression and/or upregulation of the one or more inhibitory molecule. In some embodiments, the reduction of expression and/or the inhibition of upregulation is by at least 30%, 40%, 50%, 60%, 70%, 80%, 90%, 95% or more compared to the expression or upregulation of the same inhibitory molecule in corresponding T cells that do not contain introduction of the agent when incubated under the conditions leading to expression and/or upregulation of the one or more inhibitory molecules.

As used herein, reference to a “corresponding T cell” or a “corresponding population of cells containing T cells” refers to T cells or cells obtained, isolated, generated, produced and/or incubated under the same or substantially the conditions, except that the T cells or population of T cells were not introduced with the agent. In some aspects, except for not containing introduction of the agent, such cells or T cells are treated identically or substantially identically as T cells or cells that have been introduced with the agent, such that any one or more conditions that can influence the activity or properties of the cell, including the upregulation or expression of the inhibitory molecule, is not varied or not substantially varied between the cells other than the introduction of the agent. For example, for purposes of assessing reduction in expression and/or inhibition of upregulation of one or more inhibitory molecules (e.g. PD-1 and PD-L1), T cells containing introduction of the agent and T cells not containing introduction of the agent are incubated under the same conditions known to lead to expression and/or upregulation of the one or more inhibitory molecule in T cells.

For example, in some embodiments, expression of one or more inhibitory molecules (e.g. PD-1 or PD-L1) and/or an upregulation of one or more inhibitory molecules (e.g. PD-1 or PD-L1) is reduced or inhibited compared to corresponding T cells not containing introduction of the agent, when the T cells are incubated under conditions that include the presence of antigen, which, as shown herein, rapidly induces expression or upregulation of inhibitory molecule or molecules (e.g. PD-1 or PD-L1) in cells that do not contain the introduced agent. In some embodiments, the incubation in the presence of antigen includes incubating the cells in vitro with the antigen, such as for 2 hours to 48 hours, 6 hours to 30 hours or 12 hours to 24 hours, each inclusive, or is for less than 48 hours, less than 36 hours or less than 24 hours. In some embodiments, the incubation in the presence of antigen occurs in vivo following administration of the cells to a subject resulting in exposure of the cells to specific antigen and leading to specific binding of the antigen to the cells for at least a portion of the incubation. In some embodiments, in T cells not containing the agent, expression and/or upregulation of the inhibitory molecule (e.g. PD-1 or PD-L1) is induced at least within or about within 24 hours, 2 days, 3 days, 4 days, 5 days, 6 days, 7 days, 8 days, 9 days or 10 days following administration of cells to the subject. In some embodiments, during the same period following administration to the subject of provided cells containing the introduced agent, the expression or upregulation of the inhibitory molecule or molecules is reduced or inhibited.

Methods and techniques for assessing the expression and/or levels of T cell markers, including inhibitory molecules, such as PD-1 or PD-L1, are known in the art. Antibodies and reagents for detection of such markers are well known in the art, and readily available. Assays and methods for detecting such markers include, but are not limited to, flow cytometry, including intracellular flow cytometry, ELISA, ELISPOT, cytometric bead array or other multiplex methods, Western Blot and other immunoaffinity-based methods. In some embodiments, assessing surface expression of markers on T cells includes detecting administered antigen receptor (e.g. CAR)-expressing cells in the subject after administration. It is within the level of a skilled artisan to detect antigen receptor (e.g. CAR)-expressing cells in a subject and assess levels of a surface marker. In some embodiments, antigen receptor (e.g. CAR)-expressing cells, such as cells obtained from peripheral blood of a subject, can be detected by flow cytometry or other immunoaffinity based method for expression of a marker unique to such cells, and then such cells can be co-stained for another T cell surface marker or markers, such as an inhibitory molecule (e.g. PD-1 or PD-L1). In some embodiments, T cells expressing an antigen receptor (e.g. CAR) can be generated to contain a truncated EGFR (EGFRt) as a non-immunogenic selection epitope, which then can be used as a marker to detect the such cells (see e.g. U.S. Pat. No. 8,802,374).

In some embodiments, one or more inhibitory molecules, such as PD-1 and/or PD-L1, are reduced, suppressed or disrupted in T cells, such as T cells produced by the provided methods, for a period of time that is longer than the time at which the cell is maintained or cultured ex vivo. In some aspects, the methods for producing such cells are performed so that at the time of administration of the cells to a subject and/or for a period of time subsequent to administration of the cells to the subject, the one or more inhibitory molecules, such as PD-1 or PD-L1, is reduced, suppressed or disrupted. In some embodiments, the ex vivo cultured cells are introduced with the agent no more than 2 hours, 6 hours, 12 hours, 24 hours, 2 days, 3 days or 4 days prior to administration of the cells to a subject.

In some embodiments, introduction of the agent into cells is provided to achieve transient or temporary reduction of expression of one or more inhibitory molecules, such as PD-1 or PD-L1, in the cell. In some embodiments, the transient or temporary reduction or inhibition of expression or upregulation is for at least 6 hours, 12 hours, 24 hours, 2 days, 3 days, 4 days, 5 days, 6 days, 7 days, 8 days, 9 days, 10 days, 11 days, 12 days, 13 days, 14 days or more.

In some embodiments, introduction of the agent into cells is provided to achieve conditional reduction of expression and/or inhibition of upregulation of one more inhibitory molecules, such as PD-1 or PD-L1, in the cells. In some embodiments, conditional reduction or inhibition can be inducible so that the agent is produced in the cell only in the presence of an inducer that is specific to an inducible element, such as an inducible promoter. In some embodiments, conditional reduction or inhibition can be repressible so that the agent is downregulated in the cell in the presence of a repressor that is specific to a repressible element, such as a repressible promoter. In some embodiments, the agent is operably linked to an inducible or repressible promoter to induce or repress, respectively, transcription of the DNA encoding the agent. As used herein, “operably linked” or “operably associated” includes reference to a functional linkage of at least two sequences. For example, operably linked includes linkage between a promoter and a second sequence, wherein the promoter sequence initiates and mediates transcription of the DNA sequence corresponding to the second sequence. Operably associated includes linkage between an inducing or repressing element and a promoter, wherein the inducing or repressing element acts as a transcriptional activator of the promoter.

In some embodiments, introduction of the agent into cells is provided to achieve permanent or non-transient reduction expression of one or more inhibitory molecules in the cells, such as via disruption of a gene and/or stable introduction of the one or more agents in the cell.

In some embodiments, cells provided herein include those in which expression of PD-L1 is reduced or disrupted in the cells, such as by introduction of an agent into the cell capable of reducing expression of the gene or disrupting a gene encoding PD-L1, such as CD274. In some embodiments, the reduction of expression and/or the inhibition of upregulation of PD-L1 is by at least 30%, 40%, 50%, 60%, 70%, 80%, 90%, 95% or more compared to the expression or upregulation of PD-L1 in corresponding T cells that do not contain introduction of the agent when incubated under the conditions leading to expression and/or upregulation of PD-L1. In some embodiments, the reduction or disruption of PD-L1 expression in the cell is permanent or is not-transient. In some embodiments, the reduction or disruption of PD-L1 expression in the cell is transient or conditional.

In some embodiments, cells provided herein include those in which expression of PD-1 is reduced either transiently or conditionally, and in some cases not permanently, in the cell. In some embodiments, PD-1 contributes to differentiation of memory phenotype T cells, such that a permanent reduction or disruption of the gene may have detrimental effects on CD8 memory differentiation over time. In some embodiments, the transient, such as conditional, reduction of expression and/or the inhibition of upregulation of PD-1 is by at least 30%, 40%, 50%, 60%, 70%, 80%, 90%, 95% or more compared to the expression or upregulation of PD-1 in corresponding T cells that do not contain introduction of the agent when incubated under the same conditions for the time period of the transient effect.

In some embodiments, transient or reversible repression strategies are used, such as gene knockdown using antisense, RNAi or other RNA interfering agent. As used herein, the term “RNA interfering agent” refers to a class of polynucleotides that are capable of inhibiting or down-regulating gene expression, for example by mediating RNA interference or gene silencing in a sequence-specific manner. By way of example, RNA interfering agents can include, but are not limited to dsRNAs, including siRNAs, as well as shRNAs, miRNAs. By “inhibit,” “down-regulate” or “reduce” expression, it is meant that the expression of the gene product, and/or the level of the corresponding target mRNA molecules, and/or the level of activity of one or more gene products encoded by the target mRNA, is reduced below that observed in the absence of an RNA interfering agent, i.e. baseline or control levels. In some embodiments, the percent inhibition or down regulation is about or 10%, 20%, 30%, 40%, 50%, 60%, 70%, 80%, 90%, or more. Accordingly, in some embodiments, the mRNA levels, gene product levels, or gene product activity of an “inhibited” or “reduced” or “down-regulated” target can be equal or greater than 10%, 20%, 30%, 40%, 50%, 60%, 70%, 80%, or 90%, of baseline levels, or activity.

In some embodiments, methods of producing or generating genetically engineered T cells include introducing into a population of cells containing T cells one or more nucleic acid encoding a genetically engineered antigen receptor (e.g. CAR) and an agent, for example, one or more nucleic acid molecule that is or includes or encodes an agent or agents that is an antisense, RNAi or other interfering agent specific against an inhibitory immune molecule, such as PD-1 or PD-L1. In some embodiments, the nucleic acid molecule is or includes or encodes an agent or agents that is a small interfering RNA (siRNA), a microRNA-adapted shRNA, a short hairpin RNA (shRNA), a hairpin siRNA, a precursor microRNA (pre-miRNA), pri-miRNA, or a microRNA (miRNA).

In some embodiments, the one or more agent introduced into the cell is capable of disrupting the gene encoding an inhibitory molecule, such as PD-L1. In some embodiments, disruption is by deletion, e.g., deletion of an entire gene, exon, or region, and/or replacement with an exogenous sequence, and/or by mutation, e.g., frameshift or missense mutation, within the gene, typically within an exon of the gene. In some embodiments, the disruption results in a premature stop codon being incorporated into the gene, such that the inhibitory molecule (e.g. PD-1 or PD-L1) is not expressed or is not expressed in a form that is capable of being expressed on the cells surface and/or capable of mediating cell signaling. The disruption is generally carried out at the DNA level. The disruption generally is permanent, irreversible, or not transient.

In some aspects, the disruption is carried out by gene editing, such as using a DNA binding protein or DNA-binding nucleic acid, which specifically binds to or hybridizes to the gene at a region targeted for disruption. In some aspects, the protein or nucleic acid is coupled to or complexed with a nuclease, such as in a chimeric or fusion protein. For example, in some embodiments, the disruption is effected using a fusion comprising a DNA-targeting protein and a nuclease, such as a Zinc Finger Nuclease (ZFN) or TAL-effector nuclease (TALEN), or an RNA-guided nuclease such as a clustered regularly interspersed short palindromic nucleic acid (CRISPR)-Cas system, such as CRISPR-Cas9 system, specific for the gene being disrupted. In some embodiments, methods of producing or generating genetically engineered T cells include introducing into a population of cells containing T cells one or more nucleic acid encoding a genetically engineered antigen receptor (e.g. CAR) and one or more nucleic acid encoding an agent targeting PD-L1 that is a gene editing nuclease, such as a fusion of a DNA-targeting protein and a nuclease such as a ZFN or a TALEN, or an RNA-guided nuclease such as of the CRISPR-Cas9 system, specific for PD-L1.

In some embodiments, the provided methods of reducing or inhibiting inhibitory interactions in genetically engineered cells, such as CAR-expressing cells, involve administering one or more repeat or consecutive doses of cells subsequent to administering a first dose of cells. In some cases, a first or prior dose of administered cells may eventually upregulate, following encounter with the target antigen receptor or other T cell activating stimulation, one or more inhibitory molecules, such as PD-1 and/or PD-L1, e.g., on the cell surface. Upregulation of such molecules may contribute to loss of function and/or exhaustion of the T cells and for example may impair long-term exposure to the cells. A repeat or consecutive dose(s) of cells may be used to deliver cells not expressing the inhibitory molecules, such as PD-1 and/or PD-L1, or expressing them at lower levels compared to the cells present in the subject. In some embodiments, in the consecutive dose, the inhibitory molecule(s) are not expressed or substantially expressed (or expressed to the same degree as a reference cell population) on the cells therein (or on greater than 50, 40, 30, 20, 10, or 5% of the cells therein), for example, expressed only at low levels on administered cells, such as levels that are less than or about less than 70%, 60%, 50%, 40%, 30%, 20%, 10%, 5% or less the maximal level of expression of the inhibitory molecule on the cell when stimulated under conditions that induce expression of the molecule and/or when stimulated by exposure to the antigen recognized by the CAR. In some embodiments, repeated doses of cells that do not express or do not substantially express inhibitory molecules, such as PD-1 and PD-L1, can extend the time during which functional CAR-expressing T cells, or CAR-expressing T cells with robust function, are present in the subject. In some embodiments, replenishing the army of genetically engineered T cells by administering one or more consecutive doses can lead to a greater and/or longer degree of exposure to the antigen receptor (e.g. CAR)-expressing cells and improve treatment outcomes. In some embodiments, the consecutive dose is administered at a time at which PD-L1 or PD-1 is upregulated compared to a reference level or population, such as compared to the cells in the composition of the first dose immediately prior to administration to the subject, for example, to a degree that is at least 10, 20, 30, 40, 50, 60, 70, or 80% higher surface expression as compared to the reference population.

The receptor, e.g., the CAR, expressed by the cells in the consecutive dose(s) generally specifically binds to the same antigen as the CAR of the first dose and is often the same receptor or extremely similar to the receptor in the cells of the first dose. In some embodiments, the receptor on the cells in the consecutive dose(s) is the same as or shares a large degree of identity with the receptor in the cells of the first dose.

In some embodiments, the CAR expressed by the cells of the consecutive dose contains the same scFv, the same signaling domains, and/or the same junctions as the CAR expressed by the cells of the first dose. In some embodiments, it further contains the same costimulatory, stimulatory, transmembrane, and/or other domains as that of the first dose. In some embodiments, one or more component of the CAR of the consecutive dose is distinct from the CAR of the first dose.

In some aspects of any of the provided methods, genetically engineered cells are produced or generated in ex vivo methods under conditions in which one or more inhibitory molecules, such as PD-1 and/or PD-L1, are not induced or upregulated or are not substantially induced or upregulated, or are upregulated or induced to a lesser degree as compared to other conditions. In some embodiments, the level of expression of PD-1 and/or PD-L1 on genetically engineered T cells prior to administration to a subject can be determined or monitored to confirm such cells do not express or do not substantially express the one or more inhibitory molecules. A number of well-known methods for assessing expression level of recombinant molecules may be used, such as detection by affinity-based methods, e.g., immunoaffinity-based methods, e.g., in the context of cell surface proteins, such as by flow cytometry. In some cases, expression levels can be compared to expression levels in cells stimulated under conditions known to induce expression of the molecule. For example, as described herein, conditions that induce expression of the molecule can include, in some cases, antigen stimulation through the engineered antigen receptor, such as CAR. Also, other conditions that induce T cell activation, such as stimulation through the natural TCR/CD28 signaling pathway, also can induce expression of inhibitory molecules, such as PD-1 and PD-L1 on T cells. In some embodiments, conditions are used in which PD-1 is upregulated or is upregulated to the same or similar degree as the reference conditions, but in which PD-L1 expression or upregulation is blocked not upregulated or is not substantially upregulated or is upregulated to a lesser degree than the reference conditions.

In some embodiments, the provided compositions containing genetically engineered antigen receptor cells, such as CAR-expressing cells, exhibit increased persistence when administered in vivo to a subject. In some embodiments, the persistence of genetically engineered cells, such as CAR-expressing T cells, in the subject upon administration is greater as compared to that which would be achieved by alternative methods, such as those involving administration of cells genetically engineered by methods in which T cells were not introduced with an agent that reduces or disrupts a gene involved in inhibiting the immune response, such as PD-1 and/or PD-L1. In some aspects, the persistence of provided cells, such as cells produced by the provided methods, is greater as compared to that which would be achieved by administration of a population of cells containing a genetically engineered antigen receptor, such as CAR-expressing cells, in which cells in the composition are capable of expressing or upregulating the inhibitory ligand PD-L1 in response to stimulation through the engineered and artificial receptor via specific antigen.

In some embodiments, the persistence is increased at least or at least about 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In some embodiments, the degree or extent of persistence of administered cells can be detected or quantified after administration to a subject. For example, in some aspects, quantitative PCR (qPCR) is used to assess the quantity of cells expressing the recombinant receptor (e.g., CAR-expressing cells) in the blood or serum or organ or tissue (e.g., disease site) of the subject. In some aspects, persistence is quantified as copies of DNA or plasmid encoding the receptor, e.g., CAR, per microgram of DNA, or as the number of receptor-expressing, e.g., CAR-expressing, cells per microliter of the sample, e.g., of blood or serum, or per total number of peripheral blood mononuclear cells (PBMCs) or white blood cells or T cells per microliter of the sample. In some embodiments, flow cytometric assays detecting cells expressing the receptor generally using antibodies specific for the receptors also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with the recombinant receptor (e.g. CAR-expressing cells) can be used to distinguish the administered cells from endogenous cells in a subject.

Also provided are methods and uses of the cells, such as in adoptive therapy in the treatment of cancers. Also provided are methods for engineering, preparing, and producing the cells, compositions containing the cells, and kits and devices containing and for using, producing and administering the cells. Also provided are methods, compounds, and compositions for producing the engineered cells. Provided are methods for cell isolation, genetic engineering and gene reduction or disruption. Provided are nucleic acids, such as constructs, e.g., viral vectors encoding the genetically engineered antigen receptors and/or encoding an agent for effecting reduction or disruption, and methods for introducing such nucleic acids into the cells, such as by transduction. Also provided are compositions containing the engineered cells, and methods, kits, and devices for administering the cells and compositions to subjects, such as for adoptive cell therapy. In some aspects, the cells are isolated from a subject, engineered, and administered to the same subject. In other aspects, they are isolated from one subject, engineered, and administered to another subject.

### II. Genetically Engineered Cells and T Cells

Provided are cells for adoptive cell therapy, e.g., adoptive immunotherapy, and method for producing or generating the cells. The cells include immune cells such as T cells. The cells generally are engineered by introducing one or more genetically engineered nucleic acid or product thereof. Among such products are genetically engineered antigen receptors, including engineered T cell receptors (TCRs) and functional non-TCR antigen receptors, such as chimeric antigen receptors (CARs), including activating, stimulatory, and costimulatory CARs, and combinations thereof. In some embodiments, the cells also are introduced, either simultaneously or sequentially with the nucleic acid encoding the genetically engineered antigen receptor, with a nucleic acid that is or includes or encodes an agent that is capable of reducing, suppressing or disrupting an immune inhibitory molecule, such as PD-1 or PD-L1 in the cells.

A. Cells

In some embodiments, the cells, e.g., engineered cells, are eukaryotic cells, such as mammalian cells, e.g., human cells. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MALT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, one or more of the T cell populations is enriched for or depleted of cells that are positive for (marker⁺) or express high levels (marker^{high}) of one or more particular markers, such as surface markers, or that are negative for (marker⁻) or express relatively low levels (marker^{low}) of one or more markers. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8⁺ cells or the T cells, e.g., CD3⁺ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD127). In some examples, CD8+ T cells are enriched for cells positive for CD45RO (or negative for CD45RA) and for CD62L.

In some embodiments, a CD4+ T cell population and a CD8+ T cell sub-population, e.g., a sub-population enriched for central memory (T_{QM}) cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

B. Genetically Engineered Antigen Receptors

In some embodiments, the cells comprise one or more nucleic acids introduced via genetic engineering, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

1. Chimeric Antigen Receptors (CARs)

The cells generally express recombinant receptors, such as antigen receptors including functional non-TCR antigen receptors, e.g., chimeric antigen receptors (CARs), and other antigen-binding receptors such as transgenic T cell receptors (TCRs). Also among the receptors are other chimeric receptors.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. No. 8,339,645, U.S. Pat. No. 7,446,179, US 2013/0149337, U.S. Pat. No. 7,446,190, U.S. Pat. No. 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. No. 8,339,645, U.S. Pat. No. 7,446,179, US 2013/0149337, U.S. Pat. No. 7,446,190, and U.S. Pat. No. 8,389,282. The chimeric receptors, such as CARs, generally include an extracellular antigen binding domain, such as a portion of an antibody molecule, generally a variable heavy (V_{H}) chain region and/or variable light (V_{L}) chain region of the antibody, e.g., an scFv antibody fragment.

In some embodiments, the antigen targeted by the receptor is a polypeptide. In some embodiments, it is a carbohydrate or other molecule. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Antigens targeted by the receptors in some embodiments include orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, c-Met, GD-2, and MAGE A3, CE7, Wilms Tumor 1 (WT-1), a cyclin, such as cyclin A1 (CCNA1), and/or biotinylated molecules, and/or molecules expressed by HIV, HCV, HBV or other pathogens.

In some embodiments, the CAR binds a pathogen-specific antigen. In some embodiments, the CAR is specific for viral antigens (such as HIV, HCV, HBV, etc.), bacterial antigens, and/or parasitic antigens.

In some embodiments, the antibody portion of the recombinant receptor, e.g., CAR, further includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers, e.g., hinge regions, include those described in international patent application publication number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain.

This antigen recognition domain generally is linked to one or more intracellular signaling components, such as signaling components that mimic activation through an antigen receptor complex, such as a TCR complex, and optionally associated costimulatory signals, in the case of a CAR, and/or signal via another cell surface receptor. Thus, in some embodiments, the antigen-binding component (e.g., antibody) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CDS, CD9, CD16, CD22, CD33, CD37, CD64, CD80, CD86, CD134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling domains are those that mimic or approximate a signal through a natural antigen receptor (e.g., CD3 signal), a signal through such a receptor in combination with a costimulatory receptor (e.g., CD3/CD28 signal), and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CDS, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components.

In some embodiments, the activating domain is included within one CAR, whereas the costimulatory component is provided by another CAR recognizing another antigen. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, an NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. See WO2014031687.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antibody or antibody fragment. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain. In some aspects, the transmembrane domain contains a transmembrane portion of CD28. In some embodiments, the chimeric antigen receptor contains an intracellular domain of a T cell costimulatory molecule. In some aspects, the T cell costimulatory molecule is CD28 or 41BB.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

2. TCRs

In some embodiments, the genetically engineered antigen receptors include recombinant T cell receptors (TCRs) and/or TCRs cloned from naturally occurring T cells. In some embodiments, a high-affinity T cell clone for a target antigen (e.g., a cancer antigen) is identified, isolated from a patient, and introduced into the cells. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354).

In some embodiments, after the T-cell clone is obtained, the TCR alpha and beta chains are isolated and cloned into a gene expression vector. In some embodiments, the TCR alpha and beta genes are linked via a picornavirus 2A ribosomal skip peptide so that both chains are coexpression. In some embodiments, genetic transfer of the TCR is accomplished via retroviral or lentiviral vectors, or via transposons (see, e.g., Baum et al. (2006) Molecular Therapy: The Journal of the American Society of Gene Therapy. 13:1050-1063; Frecha et al. (2010) Molecular Therapy: The Journal of the American Society of Gene Therapy. 18:1748-1757; and Hackett et al. (2010) Molecular Therapy: The Journal of the American Society of Gene Therapy. 18:674-683).

3. Multi-Targeting

In some embodiments, the cells and methods include multi-targeting strategies, such as expression of two or more genetically engineered receptors on the cell, each recognizing the same of a different antigen and typically each including a different intracellular signaling component. Such multi-targeting strategies are described, for example, in International Patent Application, Publication No.: WO 2014055668 A1 (describing combinations of activating and costimulatory CARs, e.g., targeting two different antigens present individually on off-target, e.g., normal cells, but present together only on cells of the disease or condition to be treated) and Fedorov et al., Sci. Transl. Medicine, 5(215) (December, 2013) (describing cells expressing an activating and an inhibitory CAR, such as those in which the activating CAR binds to one antigen expressed on both normal or non-diseased cells and cells of the disease or condition to be treated, and the inhibitory CAR binds to another antigen expressed only on the normal cells or cells which it is not desired to treat).

For example, in some embodiments, the cells include a receptor expressing a first genetically engineered antigen receptor (e.g., CAR or TCR) which is capable of inducing an activating signal to the cell, generally upon specific binding to the antigen recognized by the first receptor, e.g., the first antigen. In some embodiments, the cell further includes a second genetically engineered antigen receptor (e.g., CAR or TCR), e.g., a chimeric costimulatory receptor, which is capable of inducing a costimulatory signal to the immune cell, generally upon specific binding to a second antigen recognized by the second receptor. In some embodiments, the first antigen and second antigen are the same. In some embodiments, the first antigen and second antigen are different.

In some embodiments, the first and/or second genetically engineered antigen receptor (e.g. CAR or TCR) is capable of inducing an activating signal to the cell. In some embodiments, the receptor includes an intracellular signaling component containing ITAM or ITAM-like motifs. In some embodiments, the activation induced by the first receptor involves a signal transduction or change in protein expression in the cell resulting in initiation of an immune response, such as ITAM phosphorylation and/or initiation of ITAM-mediated signal transduction cascade, formation of an immunological synapse and/or clustering of molecules near the bound receptor (e.g. CD4 or CD8, etc.), activation of one or more transcription factors, such as NF-κB and/or AP-1, and/or induction of gene expression of factors such as cytokines, proliferation, and/or survival.

In some embodiments, the first and/or second receptor includes intracellular signaling domains of costimulatory receptors such as CD28, CD137 (4-1BB), OX40, and/or ICOS. In some embodiments, the first and second receptors include an intracellular signaling domain of a costimulatory receptor that are different. In one embodiment, the first receptor contains a CD28 costimulatory signaling region and the second receptor contain a 4-1BB co-stimulatory signaling region or vice versa.

In some embodiments, the first and/or second receptor includes both an intracellular signaling domain containing ITAM or ITAM-like motifs and an intracellular signaling domain of a costimulatory receptor.

In some embodiments, the first receptor contains an intracellular signaling domain containing ITAM or ITAM-like motifs and the second receptor contains an intracellular signaling domain of a costimulatory receptor. The costimulatory signal in combination with the activating signal induced in the same cell is one that results in an immune response, such as a robust and sustained immune response, such as increased gene expression, secretion of cytokines and other factors, and T cell mediated effector functions such as cell killing.

In some embodiments, neither ligation of the first receptor alone nor ligation of the second receptor alone induces a robust immune response. In some aspects, if only one receptor is ligated, the cell becomes tolerized or unresponsive to antigen, or inhibited, and/or is not induced to proliferate or secrete factors or carry out effector functions. In some such embodiments, however, when the plurality of receptors are ligated, such as upon encounter of a cell expressing the first and second antigens, a desired response is achieved, such as full immune activation or stimulation, e.g., as indicated by secretion of one or more cytokine, proliferation, persistence, and/or carrying out an immune effector function such as cytotoxic killing of a target cell.

In some embodiments, the two receptors induce, respectively, an activating and an inhibitory signal to the cell, such that binding by one of the receptor to its antigen activates the cell or induces a response, but binding by the second inhibitory receptor to its antigen induces a signal that suppresses or dampens that response. Examples are combinations of activating CARs and inhibitory CARs or iCARs. Such a strategy may be used, for example, in which the activating CAR binds an antigen expressed in a disease or condition but which is also expressed on normal cells, and the inhibitory receptor binds to a separate antigen which is expressed on the normal cells but not cells of the disease or condition.

In some embodiments, the multi-targeting strategy is employed in a case where an antigen associated with a particular disease or condition is expressed on a non-diseased cell and/or is expressed on the engineered cell itself, either transiently (e.g., upon stimulation in association with genetic engineering) or permanently. In such cases, by requiring ligation of two separate and individually specific antigen receptors, specificity, selectivity, and/or efficacy may be improved.

In some embodiments, the plurality of antigens, e.g., the first and second antigens, are expressed on the cell, tissue, or disease or condition being targeted, such as on the cancer cell. In some aspects, the cell, tissue, disease or condition is multiple myeloma or a multiple myeloma cell. In some embodiments, one or more of the plurality of antigens generally also is expressed on a cell which it is not desired to target with the cell therapy, such as a normal or non-diseased cell or tissue, and/or the engineered cells themselves. In such embodiments, by requiring ligation of multiple receptors to achieve a response of the cell, specificity and/or efficacy is achieved.

4. Vectors and Methods for Genetic Engineering

Also provided are methods, nucleic acids, compositions, and kits, for producing the genetically engineered cells. In some aspects, the genetic engineering involves introduction of a nucleic acid encoding the genetically engineered component or other component for introduction into the cell, such as a component encoding a gene-disruption protein or nucleic acid.

In some embodiments, gene transfer is accomplished by first stimulating cell growth, e.g., T cell growth, proliferation, and/or activation, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the patient to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell 2:223, 1977) which confers ganciclovir sensitivity; the cellular hypoxanthine phosphribosyltransferase (HPRT) gene, the cellular adenine phosphoribosyltransferase (APRT) gene, or bacterial cytosine deaminase, (Mullen et al., Proc. Natl. Acad. Sci. USA. 89:33 (1992)).

In some aspects, the cells further are engineered to promote expression of cytokines or other factors. Various methods for the introduction of genetically engineered components, e.g., antigen receptors, e.g., CARs, are well known and may be used with the provided methods and compositions. Exemplary methods include those for transfer of nucleic acids encoding the receptors, including via viral, e.g., retroviral or lentiviral, transduction, transposons, and electroporation.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 April 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November; 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al., (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the genetically engineered nucleic acids encoding the genetically engineered products are those described, e.g., in international patent application Publication No.: WO2014055668, and U.S. Pat. No. 7,446,190.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

Also among the additional nucleic acids are those encoding an inhibitory nucleic acid molecule, including those described below.

5. Preparation of Cells for Engineering

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques.

For example, CD3+, CD28+ T cells can be positively selected using anti-CD3/anti-CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (T_{CM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{CM}-enriched CD8+ T cells and CD4+ T cells further enhances efficacy.

In embodiments, memory T cells are present in both CD62L+ and CD62L− subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD127; in some aspects, it is based on negative selection for cells expressing or highly expressing CD45RA and/or granzyme B. In some aspects, isolation of a CD8+ population enriched for T_{CM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4+ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+ T helper cells are sorted into naïve, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L- and CD45RO.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In vitro and In vivo, p 17-25 Edited by: S. A. Brooks and U. Schumacher© Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotec, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotec), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother. 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale FACS and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the provided methods include cultivation, incubation, culture, and/or genetic engineering steps. The incubation and/or engineering may be carried out in a culture vessel, such as a unit, chamber, well, column, tube, tubing set, valve, vial, culture dish, bag, or other container for culture or cultivating cells. In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure (with or without costimulation), and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti-CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

### III. Methods for Repressing Gene Expression to Modulate PD-1 and PD-L1 Interactions Involving Genetically Engineered T Cells and Engineered Cells

In some embodiments, methods of preparing genetically engineered cells include introducing an agent that reduces or is capable of reducing expression of an immune inhibitory molecule (e.g. PD-1 or PD-L1) in the cell, which introduction can occur simultaneously or sequentially with introduction of the nucleic acid encoding the transgenic receptor, such as the CAR. In some embodiments, a nucleic acid molecule that includes, is encompassed within, or encodes the agent is introduced into the cells. Also provided are cells comprising a genetically engineered (recombinant) cell surface receptors and that have reduced expression of, or are disrupted in a gene encoding, an immune inhibitory molecule, such as PD-1 or PD-L1. In some embodiments, the cells comprise an agent, such as an inhibitory nucleic acid molecule, that reduces or represses expression of the immune inhibitory molecule.

In some embodiments, expression, activity, and/or function of one or more genes is repressed in the cell. The provided methods result in gene repression in a cell, such as in a T cell, for example in a CAR-expressing T cell. In some embodiments, also provided is a cell, such as a T cell, for example a CAR-expressing T cell, containing an agent that is capable of reducing an inhibitory effect by repressing and/or disrupting a gene in an engineered cell, such as a gene involved in inhibiting an immune response by the cell. In some embodiments, the one or more gene repressed a gene encoding PD-1 and/or PD-L1. In some embodiments, the gene or genes repressed is PDCD1 and/or CD274.

In some embodiments, the gene repression is carried out by effecting a disruption in the gene, such as a knock-out, insertion, mis sense or frameshift mutation, such as a biallelic frameshift mutation, deletion of all or part of the gene, e.g., one or more exon or portion thereof, and/or knock-in. Such disruptions in some embodiments can be effected by an agent t that includes sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas), specifically designed to be targeted to the sequence of a gene or a portion thereof. In some embodiments, such sequence-specific or targeted nucleases are encoding by an inhibitory nucleic acid molecule. In some embodiments, such nucleases can be guided or targeted by DNA-binding nucleic acid molecules, such as a guide RNA (gRNA).

In some embodiments, gene repression is carried out by effecting a reduction in expression of the immune inhibitory molecule, such as PD-1 or PD-L1. In some embodiments, such gene repression is achieved using an inhibitory nucleic acid molecule, such as by RNA interference (RNAi), short interfering RNA (siRNA), short hairpin (shRNA), micro RNA (miRNA), antisense RNA, and/or ribozymes, which can be used to selectively suppress or repress expression of the gene. siRNA technology includes that based on RNAi utilizing a double-stranded RNA molecule having a sequence homologous with the nucleotide sequence of mRNA which is transcribed from the gene, and a sequence complementary with the nucleotide sequence. siRNA generally is homologous/complementary to one region of mRNA which is transcribed from the gene, or may be siRNA including a plurality of RNA molecules which are homologous/complementary to different regions. In some embodiments, gene repression is achieved using a DNA-binding nucleic acid molecule, such as a guide RNA (gRNA), and a variant of an RNA-guided nuclease, such as an enzymatically inactive Cas9 (eiCas9) protein or a fusion protein containing eiCas9. In some embodiments, gene repression is achieved by DNA-binding targeted proteins, such as zinc finger proteins (ZFP) or fusion proteins containing ZFP. A. Reducing PD-1 or PD-L1 expression

In some embodiments, the provided methods and cells result in knockdown, such as a reduction or repression, of expression of PD-1 or PD-L1 in the cells. In some embodiments, the knockdown can be transient, such as is conditional. In some embodiments, the knockdown is non-transient or permanent.

In some embodiments, knocking down, repressing or reducing expression of PD-1 or PD-L1 can be achieved by RNA interference (RNAi). In some embodiments, RNAi can be mediated by double stranded RNA (dsRNA) molecules that have sequence-specific homology to their target nucleic acid sequences (Caplen, N. J., et al., Proc. Natl. Acad. Sci. USA 98:9742-9747 (2001)). Biochemical studies in Drosophila cell-free lysates indicate that, in some embodiments, the mediators of RNA-dependent gene silencing are 21-25 nucleotide “small interfering” RNA duplexes (siRNAs). The siRNAs can be derived from the processing of dsRNA by an RNase enzyme known as Dicer (Bernstein, E., et al., Nature 409:363-366 (2001)). siRNA duplex products can be recruited into a multi-protein siRNA complex termed RNA Induced Silencing Complex (RISC). In some embodiments, a RISC can then be guided to a target nucleic acid (suitably mRNA), where the siRNA duplex interacts in a sequence-specific way to mediate cleavage in a catalytic fashion (Bernstein, E., et al., Nature 409: 363-366 (2001); Boutla, A., et al., Curr. Biol. 11:1776-1780 (2001)). Small interfering RNAs can be synthesized and used according to procedures that are well known in the art and that will be familiar to the ordinarily skilled artisan. Small interfering RNAs comprise between about 0 to about 50 nucleotides (nt). In examples of nonlimiting embodiments, siRNAs can comprise about 5 to about 40 nt, about 5 to about 30 nt, about 10 to about 30 nt, about 15 to about 25 nt, or about 20-25 nucleotides.

In some embodiments, an RNA interfering agent is at least partly double-stranded RNA having a structure characteristic of molecules that are known in the art to mediate inhibition of gene expression through an RNAi mechanism or an RNA strand comprising at least partially complementary portions that hybridize to one another to form such a structure. When an RNA comprises complementary regions that hybridize with each other, the RNA will be said to self-hybridize. In some embodiments, an inhibitory nucleic acid, such as an RNA interfering agent, includes a portion that is substantially complementary to a target gene. In some embodiments, an RNA interfering agent optionally includes one or more nucleotide analogs or modifications. One of ordinary skill in the art will recognize that RNAi agents can include ribonucleotides, deoxyribonucleotides, nucleotide analogs, modified nucleotides or backbones, etc. In some embodiments, RNA interfering agents may be modified following transcription. In some embodiments, RNA interfering agents comprise one or more strands that hybridize or self-hybridize to form a structure that comprises a duplex portion between about 15-29 nucleotides in length, optionally having one or more mismatched or unpaired nucleotides within the duplex. In some embodiments, RNA interfering agents include short interfering RNAs (siRNAs), short hairpin RNAs (shRNAs), and other RNA species that can be processed intracellularly to produce shRNAs including, but not limited to, RNA species identical to a naturally occurring miRNA precursor or a designed precursor of an miRNA-like RNA.

In some embodiments, the term “short, interfering RNA” (siRNA) refers to a nucleic acid that includes a double-stranded portion between about 15-29 nucleotides in length and optionally further comprises a single-stranded overhang (e.g., 1-6 nucleotides in length) on either or both strands. In some embodiments, the double-stranded portion can be between 17-21 nucleotides in length, e.g., 19 nucleotides in length. In some embodiments, the overhangs are present on the 3′ end of each strand, can be 2 nucleotides long, and can be composed of DNA or nucleotide analogs. An siRNA may be formed from two RNA strands that hybridize together, or may alternatively be generated from a longer double-stranded RNA or from a single RNA strand that includes a self-hybridizing portion, such as a short hairpin RNA. One of ordinary skill in the art will appreciate that one or more mismatches or unpaired nucleotides can be present in the duplex formed by the two siRNA strands. In some embodiments, one strand of an siRNA (the “antisense” or “guide” strand) includes a portion that hybridizes with a target nucleic acid, e.g., an mRNA transcript. In some embodiments, the antisense strand is perfectly complementary to the target over about 15-29 nucleotides, sometimes between 17-21 nucleotides, e.g., 19 nucleotides, meaning that the siRNA hybridizes to the target transcript without a single mismatch over this length. However, one of ordinary skill in the art will appreciate that one or more mismatches or unpaired nucleotides may be present in a duplex formed between the siRNA strand and the target transcript.

In some embodiments, PD-L1 and/or PD-1 expression is reduced or repressed using small-hairpin RNAs (shRNAs) that target nucleic acids encoding PD-L1 or PD-1. In some embodiments, a short hairpin RNA (shRNA) is a nucleic acid molecule comprising at least two complementary portions hybridized or capable of hybridizing to form a duplex structure sufficiently long to mediate RNAi (typically between 15-29 nucleotides in length), and at least one single-stranded portion, typically between approximately 1 and 10 nucleotides in length that forms a loop connecting the ends of the two sequences that form the duplex. In some embodiments, the structure may further comprise an overhang. Suitable shRNA sequences for the knock down of a given target gene are well known in the art or can readily be determined by a person skilled in the art.

In some embodiments, the duplex formed by hybridization of self-complementary portions of the shRNA may have similar properties to those of siRNAs and, as described below, shRNAs can be processed into siRNAs by the conserved cellular RNAi machinery. Thus shRNAs can be precursors of siRNAs and can be similarly capable of inhibiting expression of a target transcript. In some embodiments, an shRNA includes a portion that hybridizes with a target nucleic acid, e.g., an mRNA transcript, and can be perfectly complementary to the target over about 15-29 nucleotides, sometimes between 17-21 nucleotides, e.g., 19 nucleotides. However, one of ordinary skill in the art will appreciate that one or more mismatches or unpaired nucleotides may be present in a duplex formed between the shRNA strand and the target transcript.

In some embodiments, the shRNA comprises a nucleotide (e.g. DNA) sequence of the structure A-B-C or C-B-A. In some embodiments, the cassette comprises at least two DNA segments A and C or C and A, wherein each of said at least two segments is under the control of a separate promoter as defined above (such as the Pol III promoter including inducible U6, H1 or the like). In the above segments: A can be a 15 to 35 bp or a 19 to 29 bp DNA sequence being at least 90%, or 100% complementary to the gene to be knocked down (e.g. PD-L1 or PD-1); B can be a spacer DNA sequence having 5 to 9 bp forming the loop of the expressed RNA hairpin molecule, and C can be a 15 to 35 or a 19 to 29 bp DNA sequence being at least 85% complementary to the sequence A.

In some embodiments, an RNA interfering agent is considered to be “targeted” to a transcript and to the gene that encodes the transcript if (1) the RNAi agent comprises a portion, e.g., a strand, that is at least approximately 80%, approximately 85%, approximately 90%, approximately 91%, approximately 92%, approximately 93%, approximately 94%, approximately 95%, approximately 96%, approximately 97%, approximately 98%, approximately 99%, or approximately 100% complementary to the transcript over a region about 15-29 nucleotides in length, e.g., a region at least approximately 15, approximately 17, approximately 18, or approximately 19 nucleotides in length; and/or (2) the Tm of a duplex formed by a stretch of 15 nucleotides of one strand of the RNAi agent and a 15 nucleotide portion of the transcript, under conditions (excluding temperature) typically found within the cytoplasm or nucleus of mammalian cells is no more than approximately 15° C. lower or no more than approximately 10° C. lower, than the Tm of a duplex that would be formed by the same 15 nucleotides of the RNA interfering agent and its exact complement; and/or (3) the stability of the transcript is reduced in the presence of the RNA interfering agent as compared with its absence. In some embodiments, an RNA interfering agent targeted to a transcript can also considered targeted to the gene that encodes and directs synthesis of the transcript. In some embodiments, a target region can be a region of a target transcript that hybridizes with an antisense strand of an RNA interfering agent. In some embodiments, a target transcript can be any RNA that is a target for inhibition by RNA interference.

In some embodiments, siRNA selectively suppresses the expression of PD-L1 and/or PD-1. In addition, all of the nucleotide sequences of siRNA may be derived from the nucleotide sequence of the mRNA of PD-L1 and/or PD-1, or a part thereof may be derived from the nucleotide sequence.

In some embodiments, the siRNA can be comprised of ribonucleotides, and a part thereof may include nucleotides other than ribonucleotides, for example, deoxyribonucleotides, a derivative of deoxyribonucleotides, a derivative of ribonucleotides, etc. The siRNA can be synthesized by a known chemical synthesis method, but the method is not particularly limited. In some embodiments, it may be enzymatically (e.g., using an RNA polymerase) prepared using a suitable template nucleic acid. In some embodiments, the siRNA may be in the form of single-stranded RNA which can form a duplex in the molecule, and single-stranded RNA with a stem-loop structure (short hairpin structure: sh structure) having the siRNA part as a stem and an arbitrary sequence as a loop (shRNA). In some embodiments, a sequence of 1 to 30 nucleotides, 1 to 25 nucleotides, or 5 to 22 nucleotides can be used as the arbitrary sequence.

The sequence of the siRNA can be appropriately designed based on a gene sequence whose expression is desired to be suppressed. Many siRNA design algorithms have been reported (see, e.g., WO 2004/0455543, and WO 2004/048566), and a commercially available software can also be used. In addition, there are many companies which design siRNA from information of a gene sequence whose expression is desired to be suppressed, and synthesize and provide the siRNA. Therefore, a person skilled in the art can easily obtain the siRNA based on the gene sequence whose expression is desired to be suppressed. In some embodiments, any siRNA which selectively suppresses expression of PD-L1 and/or PD-1 can be generated or used. For example, siRNA including the nucleotide sequence of any of SEQ ID NOS: 1-5 can be used for PD-L1, and siRNA including the nucleotide sequence of SEQ ID NO: 6 can be used for PD-1. Additional exemplary siRNA sequences directed against PD-L1 can be found in US Patent Application Publication No. 20140148497, herein incorporated by reference.

In some embodiments, shRNA and siRNA segments may further comprise stop and/or polyadenylation sequences.

In some embodiments, an antisense nucleotide can be used for suppressing the expression of PD-L1 and/or PD-1. In some embodiments, the antisense nucleotide can be used for suppressing the expression of a protein, for example, by directly interfering with translation of the mRNA molecule of PD-L1 and/or PD-1PD-1, by degradation of mRNA by an RNA degradation enzyme H, by interfering with the 5′ capping of mRNA, by masking the 5′ cap, by preventing binding of a translation factor with mRNA, or by inhibiting polyadenylation of mRNA. In some embodiments, the suppression of the expression of a protein can occur by hybridization between an antisense nucleotide and the mRNA of PD-L1 and/or PD-1. In some embodiments, a specific targeting site on the mRNA is selected as a target of the antisense nucleotide in order to reduce stability of, or degrade mRNA. In some embodiments, when one or more target sites are identified, a nucleotide having a nucleotide sequence sufficiently complementary with the target site (that is, which hybridizes sufficiently and with sufficient specificity under the physiological conditions) can be designed. In some embodiments, the antisense nucleotide can have, for example, a chain length of 8 to 100 nucleotides, 10 to 80 nucleotides, or 14 to 35 nucleotides.

In some embodiments, methods of introduction or delivery into a cell can be the same or similar to methods as described above for introduction of a nucleic acid encoding a genetically engineered antigen receptor into a cell. In some embodiments, expression of an inhibitory nucleic acid, such as an shRNA or siRNA, in cells, e.g. T cells, can be achieved using any conventional expression system, e.g., a lentiviral expression system. In some embodiments, the RNA can be a component of a viral vector. In some embodiments, the viral vector comprises an oligonucleotide that inhibits expression of PD-1 or PD-L1, or encodes a shRNA or other inhibitory nucleic acid having such capability. In some embodiments, the viral vector is a lentivirus vector. In some embodiments, the lentivirus vector is an integrating lentivirus vector.

In some embodiments, suitable promoters include, for example, RNA polymerase (pol) III promoters including, but not limited to, the (human and murine) U6 promoters, the (human and murine) H1 promoters, and the (human and murine) 7SK promoters. In some embodiments, a hybrid promoter also can be prepared that contains elements derived from, for example, distinct types of RNA polymerase (pol) III promoters. In some embodiments, modified promoters that contain sequence elements derived from two or more naturally occurring promoter sequences can be combined by the skilled person to effect transcription under a desired set of conditions or in a specific context. For example, the human and murine U6 RNA polymerase (pol) III and H1 RNA pol III promoters are well characterized. One skilled in the art will be able to select and/or modify the promoter that is most effective for the desired application and cell type so as to optimize modulation of the expression of one or more genes. In some embodiments, the promoter sequence can be one that does not occur in nature, so long as it functions in a eukaryotic cell, such as, for example, a mammalian cell.

In some embodiments, an exemplary delivery vehicle is a nanoparticle, e.g., a liposome or other suitable sub-micron sized delivery system. In some embodiments, the use of lipid formulations is contemplated for the introduction of the nucleic acids into a cell. The lipid particle may be a nucleic acid-lipid particle, which may be formed from a cationic lipid, a non-cationic lipid, and optionally a conjugated lipid that prevents aggregation of the particle. The nucleic acid may be encapsulated in the lipid portion of the particle, thereby protecting it from enzymatic degradation. A stable nucleic acid-lipid particle can be a particle made from lipids (e.g., a cationic lipid, a non-cationic lipid, and optionally a conjugated lipid that prevents aggregation of the particle), wherein the nucleic acid is fully encapsulated within the lipid.

In some embodiments, the lipid particles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the lipid particles are substantially non-toxic. In some embodiments, nucleic acids, when present in the lipid particles of the present invention, can be resistant in aqueous solution to degradation with a nuclease.

In some embodiments, a lipid particle provides a nucleic acid with full encapsulation, partial encapsulation, or both. In some embodiments, the nucleic acid is fully encapsulated in the lipid particle to form a nucleic acid-lipid particle.

In some embodiments, a conjugated lipid inhibits aggregation of lipid particles, including, polyethylene glycol (PEG)-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides, cationic PEG lipids, polyoxazoline (POZ)-lipid conjugates (e.g., POZ-DAA conjugates; polyamide oligomers (e.g., ATTA-lipid conjugates), and mixtures thereof. In some embodiments, PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In some embodiments, non-ester containing linker moieties, such as amides or carbamates, are used.

In some embodiments, an amphipathic lipid can have a hydrophobic portion that orients into a hydrophobic phase, and a hydrophilic portion orients toward the aqueous phase. In some embodiments, hydrophilic characteristics derive from the presence of polar or charged groups such as carbohydrates, phosphate, carboxylic, sulfato, amino, sulfhydryl, nitro, hydroxyl, and other like groups. In some embodiments, hydrophobicity can be conferred by the inclusion of apolar groups that include, but are not limited to, long-chain saturated and unsaturated aliphatic hydrocarbon groups and such groups substituted by one or more aromatic, cycloaliphatic, or heterocyclic group(s). Examples of amphipathic compounds include, but are not limited to, phospholipids, aminolipids, and sphingolipids.

Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and (3-acyloxyacids, are also within the group designated as amphipathic lipids. Additionally, the amphipathic lipids described above can be mixed with other lipids including triglycerides and sterols.

In some embodiments, a neutral lipid exists either in an uncharged or neutral zwitterionic form at a selected pH. In some embodiments, at physiological pH, such lipids include, for example, diacylphosphatidylcholine, diacylphosphatidylethanolamine, ceramide, sphingomyelin, cephalin, cholesterol, cerebrosides, and diacylglycerols.

In some embodiments, a non-cationic lipid may be any amphipathic lipid as well as any other neutral lipid or anionic lipid.

In some embodiments, an anionic lipid is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

In some embodiments, a hydrophobic lipid has apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N—N-dialkylamino, 1,2-diacyloxy-3-aminopropane, and 1,2-dialkyl-3-aminopropane. In some embodiments, the nucleic acid-lipid particle comprises: (a) a nucleic acid (e.g., an interfering RNA); (b) a cationic lipid comprising from about 50 mol % to about 65 mol % of the total lipid present in the particle; (c) a non-cationic lipid comprising from about 25 mol % to about 45 mol % of the total lipid present in the particle; and (d) a conjugated lipid that inhibits aggregation of particles comprising from about 5 mol % to about 10 mol % of the total lipid present in the particle.

In some embodiments, the nucleic acid-lipid particle comprises: (a) a nucleic acid (e.g., an interfering RNA); (b) a cationic lipid comprising from about 50 mol % to about 60 mol % of the total lipid present in the particle; (c) a mixture of a phospholipid and cholesterol or a derivative thereof comprising from about 35 mol % to about 45 mol % of the total lipid present in the particle; and (d) a PEG-lipid conjugate comprising from about 5 mol % to about 10 mol % of the total lipid present in the particle.

In some embodiments, the nucleic acid-lipid particle comprises: (a) a nucleic acid (e.g., an interfering RNA); (b) a cationic lipid comprising from about 55 mol % to about 65 mol % of the total lipid present in the particle; (c) cholesterol or a derivative thereof comprising from about 30 mol % to about 40 mol % of the total lipid present in the particle; and (d) a PEG-lipid conjugate comprising from about 5 mol % to about 10 mol % of the total lipid present in the particle. In some embodiments, a CRISPR/Cas system can be used for knocking down, such as reducing or suppressing, the expression of PD-L1 and/or PD-1 (see, e.g., WO2015/161276). Exemplary features of CRISPR/Cas systems are described below and can be adapted for use in reducing or suppressing expression of a molecule, rather than disrupting or deleting a gene encoding the molecule, by using an enzymatically inactive nuclease. In some embodiments, a guide RNA (gRNA) targeting a gene encoding PD-L1 or PD-1, such as the CD274 or PDCD1 gene, or the promoter, enhancer or other cis- or trans-acting regulatory regions, can be introduced in combination with a modified Cas9 protein or a fusion protein containing the modified Cas9 protein, to suppress the expression of, e.g., knock-down, of the gene(s). In some embodiments, the Cas9 molecule is an enzymatically inactive Cas9 (eiCas9) molecule, which comprises a mutation, e.g., a point mutation, that causes the Cas9 molecule to be inactive, e.g., a mutation that eliminates or substantially reduces the Cas9 molecule cleavage activity. In some embodiments, the eiCas9 molecule is fused, directly or indirectly to, a transcription activator or repressor protein.

In some embodiments, the promoter region of the PDCD1 or CD274 gene is targeted to knockdown expression of PDCD1 or CD274. A targeted knockdown approach reduces or eliminates expression of the functional PDCD1 or CD274 gene product. In some embodiments, targeted knockdown is mediated by targeting an enzymatically inactive Cas9 (eiCas9) or an eiCas9 fused to a transcription repressor domain or chromatin modifying protein to alter transcription, e.g., to block, reduce, interfere with, or decrease transcription, of the PDCD1 and/or CD274 genes. gRNA targeting a target sequence in or near the PDCD1 or CD274 genes, if targeted by an eiCas9 or an eiCas9 fusion protein, results in reduction or elimination of expression of functional PDCD1 or CD274 gene product, such as PD-1 or PD-L1. In some embodiments, transcription is reduced or eliminated.

In some embodiments, a targeting domain of the gRNA molecule is configured to target an enzymatically inactive Cas9 (eiCas9) or an eiCas9 fusion protein (e.g., an eiCas9 fused to a transcription repressor domain), sufficiently close to a target sequence in the genome to reduce, decrease or repress expression of the PDCD1 or CD274 gene. In some embodiments, an eiCas9 is fused to a transcription repressor domain or chromatin modifying protein to alter transcription, e.g., to block, reduce, interfere with or decrease transcription, of the PDCD1 or CD274 genes. In some embodiments, one or more eiCas9s may be used to block binding of one or more endogenous transcription factors. In another embodiment, an eiCas9 can be fused to a chromatin modifying protein. Altering chromatin status can result in decreased expression of the target gene. One or more eiCas9s fused to one or more chromatin modifying proteins may be used to alter chromatin status.

In some embodiments, the targeting domain is configured to target the promoter region of the PDCD1 or CD274 gene to block transcription initiation, binding of one or more transcription enhancers or activators, and/or RNA polymerase. One or more gRNA can be used to target an eiCas9 to the promoter region of the PDCD1 and/or CD274 genes. In some embodiments, one or more regions of PDCD1 and/or CD274 can be targeted.

In some embodiments, a complex of the PD-L1 or PD-1 targeting CRISPR gRNA and the enzymatically inactive nuclease, e.g. iCas9 or eiCas9 fusion protein, can be introduced into a cell by methods known to a skilled artisan, including those described below in connection with CRISPR/Cas systems. In some embodiments, the CRISPR gRNA and enzymatically inactive nuclease, e.g. iCas9 or eiCas9 fusion protein, is transiently introduced to the cell, e.g., by transient introduction of the ribonucleoprotein complex (RNP) complex. In some embodiments, nucleic acid molecules encoding the gRNA and/or eiCas9 are introduced to the cell using any conventional expression system, e.g., a lentiviral expression system. In some embodiments, methods of introduction or delivery into a cell can be the same or similar to the methods as described below for introduction of a nucleic acid-protein complex, such as a ribonucleoprotein (RNP) complex) into a cell.

In some embodiments, gene knockdown is achieved by DNA-binding targeted proteins, such as zinc finger proteins (ZFP) or fusion proteins containing ZFP, that target genes encoding PD-L1 or PD-1. In some embodiments, a DNA-binding proteins, such as a ZFP, can effect target gene repression by interfering with or inhibiting the expression of the target gene. Exemplary features of DNA-binding proteins, including ZFPs, are described below and can be adapted for use in reducing or suppressing expression of a molecule, rather than disrupting or deleting a gene encoding the molecule, by introduction without the effector protein (e.g. endonuclease, such as a zinc finger nuclease (ZFN)).

B. Knockout of PD-1 or PD-L1 Expression

In some aspects, the knockout, such as disruption of, genes encoding PD-1 and/or PD-L1, such as PDCD1 and/or CD274, is carried out by gene editing, such as using a DNA binding protein or DNA-binding nucleic acid, which specifically binds to or hybridizes to the gene at a region targeted for disruption. In some aspects, the protein or nucleic acid is coupled to or complexed with a gene editing nuclease, such as in a chimeric or fusion protein. For example, in some embodiments, the disruption is effected using a fusion comprising a DNA-targeting protein and a nuclease, such as a Zinc Finger Nuclease (ZFN) or TAL-effector nuclease (TALEN), or an RNA-guided nuclease such as a clustered regularly interspersed short palindromic nucleic acid (CRISPR)-Cas system, such as CRISPR-Cas9 system, specific for the gene being disrupted. In some embodiments, gene editing results in a genomic disruption or knock-out of genes encoding PD-1 and/or PD-L1, such as PDCD1 and/or CD274.

In some embodiments, the repression is achieved using a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease.

Zinc finger, TALE, and CRISPR system binding domains can be engineered to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073 and US20140120622.

In some embodiments, the DNA-targeting molecule, complex, or combination contains a DNA-binding molecule and one or more additional domain, such as an effector domain to facilitate the repression or disruption of the gene. For example, in some embodiments, the gene disruption or repression is carried out by fusion proteins that comprise DNA-binding proteins and a heterologous regulatory domain or functional fragment thereof. In some aspects, domains include, e.g., transcription factor domains such as activators, repressors, co-activators, co-repressors, silencers, oncogenes, DNA repair enzymes and their associated factors and modifiers, DNA rearrangement enzymes and their associated factors and modifiers, chromatin associated proteins and their modifiers, e.g. kinases, acetylases and deacetylases, and DNA modifying enzymes, e.g. methyltransferases, topoisomerases, helicases, ligases, kinases, phosphatases, polymerases, endonucleases, and their associated factors and modifiers. See, for example, U.S. Patent Application Publication Nos. 20050064474; 20060188987 and 2007/0218528, incorporated by reference in their entireties herein, for details regarding fusions of DNA-binding domains and nuclease cleavage domains. In some aspects, the additional domain is a nuclease domain. Thus, in some embodiments, gene disruption is facilitated by gene or genome editing, using engineered proteins, such as gene editing nucleases and gene editing nuclease-containing complexes or fusion proteins, composed of sequence-specific DNA-binding domains fused to or complexed with non-specific DNA-cleavage molecules such as nucleases.

In some aspects, these targeted chimeric nucleases or nuclease-containing complexes carry out precise genetic modifications by inducing targeted double-stranded breaks or single-stranded breaks, stimulating the cellular DNA-repair mechanisms, including error-prone non-homologous end joining (NHEJ) and homology-directed repair (HDR). In some embodiments the nuclease is an endonuclease, such as a zinc finger nuclease (ZFN), TALE nuclease (TALEN), an RNA-guided endonuclease (RGEN), such as a CRISPR-associated (Cas) protein, or a meganuclease.

In some embodiments, a donor nucleic acid, e.g., a donor plasmid or nucleic acid encoding the genetically engineered antigen receptor, is provided and is inserted by HDR at the site of gene editing following the introduction of the DSBs. Thus, in some embodiments, the disruption of the gene and the introduction of the antigen receptor, e.g., CAR, are carried out simultaneously, whereby the gene is disrupted in part by knock-in or insertion of the CAR-encoding nucleic acid.

In some embodiments, no donor nucleic acid is provided. In some aspects, NHEJ-mediated repair following introduction of DSBs results in insertion or deletion mutations that can cause gene disruption, e.g., by creating missense mutations or frameshifts.

1. ZFPs and ZFNs; TALs, TALEs, and TALENs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs. See Lloyd et al., Frontiers in Immunology, 4(221), 1-7 (2013).

In some embodiments, the DNA-targeting molecule comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

In some aspects, repression of the gene is carried out by contacting a first target site in the gene with a first ZFP, thereby repressing the gene. In some embodiments, the target site in the gene is contacted with a fusion ZFP comprising six fingers and the regulatory domain, thereby inhibiting expression of the gene.

In some embodiments, the step of contacting further comprises contacting a second target site in the gene with a second ZFP. In some aspects, the first and second target sites are adjacent. In some embodiments, the first and second ZFPs are covalently linked. In some aspects, the first ZFP is a fusion protein comprising a regulatory domain or at least two regulatory domains. In some embodiments, the first and second ZFPs are fusion proteins, each comprising a regulatory domain or each comprising at least two regulatory domains. In some embodiments, the regulatory domain is a transcriptional repressor, a transcriptional activator, an endonuclease, a methyl transferase, a histone acetyltransferase, or a histone deacetylase.

In some embodiments, the ZFP is encoded by a ZFP nucleic acid operably linked to a promoter. In some aspects, the method further comprises the step of first administering the nucleic acid to the cell in a lipid:nucleic acid complex or as naked nucleic acid. In some embodiments, the ZFP is encoded by an expression vector comprising a ZFP nucleic acid operably linked to a promoter. In some embodiments, the ZFP is encoded by a nucleic acid operably linked to an inducible promoter. In some aspects, the ZFP is encoded by a nucleic acid operably linked to a weak promoter.

In some embodiments, the target site is upstream of a transcription initiation site of the gene. In some aspects, the target site is adjacent to a transcription initiation site of the gene. In some aspects, the target site is adjacent to an RNA polymerase pause site downstream of a transcription initiation site of the gene.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type IIS restriction enzyme and one or more zinc finger binding domains, which may or may not be engineered. In some embodiments, the cleavage domain is from the Type IIS restriction endonuclease Fok I. Fok I generally catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982.

In some embodiments, ZFNs target a gene encoding an immune inhibitory molecule, such as a gene encoding PD-1 and/or PD-L1. In particular embodiments, a ZFN targets a gene encoding PD-L1. In some aspects, the ZFNs efficiently generate a double strand break (DSB), for example at a predetermined site in the coding region of the gene. Typical regions targeted include exons, regions encoding N-terminal regions, first exon, second exon, and promoter or enhancer regions. In some embodiments, transient expression of the ZFNs promotes highly efficient and permanent disruption of the target gene in the engineered cells. In particular, in some embodiments, delivery of the ZFNs results in the permanent disruption of the gene with efficiencies surpassing 50%.

Many gene-specific engineered zinc fingers are available commercially. For example, Sangamo Biosciences (Richmond, Calif., USA) has developed a platform (CompoZr) for zinc-finger construction in partnership with Sigma-Aldrich (St. Louis, Mo., USA), allowing investigators to bypass zinc-finger construction and validation altogether, and provides specifically targeted zinc fingers for thousands of proteins. Gaj et al., Trends in Biotechnology, 2013, 31(7), 397-405. In some embodiments, commercially available zinc fingers are used or are custom designed. (See, for example, Sigma-Aldrich catalog numbers CSTZFND, CSTZFN, CTI1-1KT, and PZD0020).

2. TALEs and TALENs

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NG binds to T and non-canonical (atypical) RVDs are also known. See, U.S. Patent Publication No. 20110301073. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE-nuclease (TALEN). In some aspects the TALEN is a fusion protein comprising a DNA-binding domain derived from a TALE and a nuclease catalytic domain to cleave a nucleic acid target sequence. In some embodiments, the TALE DNA-binding domain has been engineered to bind a target sequence within genes that encode the target antigen and/or the immunosuppressive molecule. For example, in some aspects, the TALE DNA-binding domain may target a gene encoding an immune inhibitory molecule, such as a gene encoding PD-1 and/or PD-L1. In particular embodiments, a TALE DNA-binding domain targets a gene encoding a PD-L1, such as CD274.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation (Critchlow and Jackson, Trends Biochem Sci. 1998 October; 23(10):394-8) or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. (Gaj et al., Trends in Biotechnology, 2013, 31(7), 397-405). A library of TALENs targeting 18,740 human protein-coding genes has been constructed (Kim et al., Nature Biotechnology. 31, 251-258 (2013)). Custom-designed TALE arrays are commercially available through Cellectis Bioresearch (Paris, France), Transposagen Biopharmaceuticals (Lexington, Ky., USA), and Life Technologies (Grand Island, N.Y., USA). Specifically, TALENs that target PD-1 are commercially available (See Gencopoeia, catalog numbers HTN212662-1, HTN212662-2, and HTN212662-3, available on the World Wide Web at www.genecopoeia.com/product/search/detail.php?prt=26&cid=&key=HTN212662). Exemplary molecules are described, e.g., in U.S. Patent Publication Nos. US 2014/0120622, and 2013/0315884). See also http://www.e-talen.org/E-TALEN/and Heigwer et al., Nucleic Acids Res. 41(20):e190 (2013).

In some embodiments the TALENs are introduced as transgenes encoded by one or more plasmid vectors. In some aspects, the plasmid vector can contain a selection marker which provides for identification and/or selection of cells which received said vector.

3. RGENs (CRISPR/Cas Systems)

In some embodiments, the repression is carried out using one or more DNA-binding nucleic acids, such as disruption via an RNA-guided endonuclease (RGEN), or other form of repression by another RNA-guided effector molecule. For example, in some embodiments, the repression is carried out using clustered regularly interspaced short palindromic repeats (CRISPR) and CRISPR-associated (Cas) proteins. See Sander and Joung, Nature Biotechnology, 32(4): 347-355.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In some embodiments, the CRISPR/Cas nuclease or CRISPR/Cas nuclease system includes a non-coding RNA molecule (guide) RNA (gRNA), whose sequence-specifically binds to DNA, and a Cas protein (e.g., Cas9), with nuclease functionality (e.g., two nuclease domains), or a variant thereof.

In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes or Staphylococcus aureus In some embodiments, Cas9 nuclease (e.g., that encoded by mRNA from Staphylococcus aureus or from Streptococcus pyogenes, e.g. pCW-Cas9, Addgene #50661, Wang et al. (2014) Science, 3:343-80-4; or nuclease or nickase lentiviral vectors available from Applied Biological Materials (ABM; Canada) as Cat. No. K002, K003, K005 or K006) and a guide RNA specific to the target gene (e.g. PDCD1 gene, which encodes PD-1, or the CD274 gene, which encodes PD-L1) are introduced into cells.

In general, a CRISPR system is characterized by elements that promote the formation of a CRISPR complex at the site of a target sequence. In some embodiments, the target sequence or target site is a gene encoding an immune inhibitory molecule, such as a gene encoding PD-1 or PD-L1. For example, the target sequence is in or near the PDCD1 gene, which encodes PD-1, or the CD274 gene, which encodes PD-L1. In particular embodiments, target sequence or target site is a gene encoding PD-L1, such as CD274. Typically, in the context of formation of a CRISPR complex, “target sequence” generally refers to a sequence, e.g., a gene or a genomic sequence, to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex. In some embodiments, a guide sequence is selected to reduce the degree of secondary structure within the guide sequence. Secondary structure may be determined by any suitable polynucleotide folding algorithm.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid, such as the target sequence in the CD274 or PDCD1 gene.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of the CRISPR/Cas complex to a target sequence may be assessed by any suitable assay. For example, the components of the CRISPR/Cas system sufficient to form the CRISPR/Cas complex, including the guide sequence to be tested, may be provided to the cell having the corresponding target sequence, such as by transfection with vectors encoding the components of the CRISPR/Cas complex, followed by an assessment of preferential cleavage within the target sequence, such as by Surveyor assay as described herein. Similarly, cleavage of a target polynucleotide sequence may be evaluated in a test tube by providing the target sequence, components of the CRISPR/Cas complex, including the guide sequence to be tested and a control guide sequence different from the test guide sequence, and comparing binding or rate of cleavage at the target sequence between the test and control guide sequence reactions.

In some embodiments, a Cas nuclease and gRNA (e.g. including a fusion of crRNA specific for the target sequence and fixed tracrRNA) are introduced into the cell. In general, target sites at the 5′ end of the gRNA target the Cas nuclease to the target site, e.g., the gene, using complementary base pairing. In some embodiments, the target site is selected based on its location immediately 5′ of a protospacer adjacent motif (PAM) sequence, such as typically NGG, or NAG. In this respect, the gRNA is targeted to the desired sequence by modifying the first 20 nucleotides of the guide RNA to correspond to the target DNA sequence.

In some embodiments, the target sequence is at or near gene encoding PD-L1 or PD-1, such as the CD274 or the PDCD1 gene. In some embodiments, the target nucleic acid complementary to the targeting domain is located at an early coding region of a gene of interest, such as CD274 or PDCD1. Targeting of the early coding region can be used to knockout (i.e., eliminate expression of) the gene of interest. In some embodiments, the early coding region of a gene of interest includes sequence immediately following a start codon (e.g., AUG), or within 500 bp of the start codon (e.g., less than 500, 450, 400, 350, 300, 250, 200, 150, 100 or 50 bp). In some embodiments, the target sequence is within 200, 150 or 100 bp of the start codon of CD274 or PDCD1. Targeting of the promoter region or regions near the transcription start site can be used to knockdown (i.e., reduce the expression of) the gene of interest. For example, regions near the transcription start site can include regions within 500 bp upstream of the transcription start site (e.g., less than 500, 450, 400, 350, 300, 250, 200, 150, 100 or 50 bp). In some embodiments, the target sequence can be within the promoter, enhancer or other cis- or trans-acting regulatory regions.

It is within the level of a skilled artisan to design or identify a gRNA sequence that is or comprises a sequence targeting CD274 or PDCD1, including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; http://www.e-crisp.org/E-CRISP/; http://crispr.mit.edu/; https://www.dna20.com/eCommerce/cas9/input). In some embodiments, the gRNA sequence is or comprises a sequence with minimal off-target binding to a non-target gene.

Exemplary target sequences in PDCD1 that are complementary to gRNA targeting domain sequences are set forth in SEQ ID NOS: 13-18. Exemplary target sequences in CD274 that are complementary to gRNA targeting domain sequences are set forth in SEQ ID NOS: 19-24. In some embodiments, the targeting domain against the PDCD1 gene can comprise a sequence that is the same as, or differs by no more than 1, 2, 3, 4, or 5 nucleotides from, any exemplary targeting domain of gRNA sequence described, for example, in international patent application publication number WO2015/161276.

In some embodiments, the CRISPR system induces double stranded breaks (DSBs) at the target site, followed by disruptions as discussed herein. In other embodiments, Cas9 variants, deemed “nickases” are used to nick a single strand at the target site. In some aspects, paired nickases are used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

In some embodiments, disruption includes insertion of a sequence into the gene. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence.

In some embodiments, a tracr sequence also may be included, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of the CRISPR complex. As with the target sequence, in some embodiments, complete complementarity is not necessarily needed. In some embodiments, the tracr sequence has at least 50%, 60%, 70%, 80%, 90%, 95% or 99% of sequence complementarity along the length of the tracr mate sequence when optimally aligned.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences.

Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In some aspects, loop forming sequences for use in hairpin structures are four nucleotides in length, and have the sequence GAAA. However, longer or shorter loop sequences may be used, as may alternative sequences. In some embodiments, the sequences include a nucleotide triplet (for example, AAA), and an additional nucleotide (for example C or G). Examples of loop forming sequences include CAAA and AAAG. In some embodiments, the transcript or transcribed polynucleotide sequence has at least two or more hairpins. In some embodiments, the transcript has two, three, four or five hairpins. In a further embodiment, the transcript has at most five hairpins. In some embodiments, the single transcript further includes a transcription termination sequence, such as a polyT sequence, for example six T nucleotides.

In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of the CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to the cell.

In some embodiments, a vector comprises a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. Pyogenes, S. aureus or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A; SEQ ID NO:12) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ.

In some embodiments, Cas9 or split Cas9 lacks endonuclease activity. In some embodiments, the resulting Cas9 or split Cas9 is co-expressed with guide RNA designed to comprise a complementary sequence of the target nucleic acid sequence, for example, a gene encoding PD-L1 or PD-1. In some embodiments, expression of Cas9 lacking endonuclease activity yields specific silencing or reduction of the gene of interest. This system is named CRISPR interference (CRISPRi) (Qi, Larson et al. 2013). In some embodiments, the silencing may occur at the transcriptional or the translational step. In some embodiments, the silencing may occur by directly blocking transcription, for example by blocking transcription elongation or by targeting key cis-acting motifs within any promoter, sterically blocking the association of their cognate trans-acting transcription factors. In some embodiments, the Cas9 lacking endonuclease activity comprises both non-functional HNH and RuvC domains. In some embodiments, the Cas9 or split Cas9 polypeptide comprises inactivating mutations in the catalytic residues of both the RuvC-like and HNH domains. For example, the catalytic residues required for cleavage Cas9 activity can be D10, D31, H840, H865, H868, N882 and N891 of Cas9 of S. pyogenes (COG3513—SEQ ID NO:11) or aligned positions using CLUSTALW method on homologues of Cas Family members. In some embodiments, the residues comprised in HNH or RuvC motifs can be those described in the above paragraph. In some embodiments, any of these residues can be replaced by any one of the other amino acids, for example by an alanine residue. In some embodiments, mutation in the catalytic residues means either substitution by another amino acids, or deletion or addition of amino acids that cause the inactivation of at least one of the catalytic domain of Cas9.

Non-limiting examples of mutations in a Cas9 protein are known in the art (see e.g. WO2015/161276), any of which can be included in a CRISPR/Cas9 system in accord with the provided methods.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. In some embodiments, one or more codons (e.g. 1, 2, 3, 4, 5, 10, 15, 20, 25, 50, or more, or all codons) in a sequence encoding the CRISPR enzyme correspond to the most frequently used codon for a particular amino acid.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US20110059502, incorporated herein by reference. In some embodiments, a tagged CRISPR enzyme is used to identify the location of a target sequence.

In some embodiments, a CRISPR enzyme in combination with (and optionally complexed with) a guide sequence is delivered to the cell. In some embodiments, methods for introducing a protein component into a cell according to the present disclosure (e.g. Cas9/gRNA RNPs) may be via physical delivery methods (e.g. electroporation, particle gun, Calcium Phosphate transfection, cell compression or squeezing), liposomes or nanoparticles.

Commercially available kits, gRNA vectors and donor vectors, for knockout of PD-1 via CRISPR are available, for example, from OriGene. See www.origene.com/CRISPR-CAS9/Product.aspx?SKU=KN210364; catalog numbers KN210364G1, KN210364G2, KN210364D. Likewise, commercially available kits, gRNA vectors and donor vectors, for knockout of PD-L1 via CRISPR are available, for example, from OriGene. See www.origene.com/CRISPR-CAS9/Product.aspx?SKU=KN213071; catalog numbers KN213071G1, KN213071G2, KN213071D.

In some aspects, target polynucleotides, such as genes encoding PD-1 or PD-L1, are modified in the cell in which the CRISPR complex is introduced. In some embodiments, the method comprises allowing the CRISPR complex to bind to the target polynucleotide to effect cleavage of said target polynucleotide thereby modifying the target polynucleotide, wherein the CRISPR complex comprises the CRISPR enzyme complexed with a guide sequence that hybridizes to a target sequence within said target polynucleotide, wherein said guide sequence is linked to a tracr mate sequence which in turn hybridizes to a tracr sequence.

In some embodiments, the method comprises allowing the CRISPR complex to bind to the polynucleotide such that said binding results in increased or decreased expression of said polynucleotide; wherein the CRISPR complex comprises a CRISPR enzyme complexed with a guide sequence that hybridizes to a target sequence within said polynucleotide, wherein said guide sequence is linked to a tracr mate sequence which in turn hybridizes to a tracr sequence.

D. Conditional Gene Suppression Systems

In some embodiments, the deletion, knockout, disruption, reduction of expression, disruption of expression, inhibition of upregulation and/or inhibition of function of genes encoding PD-1 or PD-L1, or PD-1 or PD-L1 molecules, is conditional. In some embodiments, conditional suppression of genes, such as genes encoding PD-1 and/or PD-L1, may be initiated or induced upon a decline in persistence of administered cells engineered with an antigen receptor (e.g. CAR) and/or upon such cells exhibiting an exhaustive phenotype. In some embodiments, conditional suppression may facilitate therapeutic applications by resulting in cells that exhibit an increased duration of exposure and/or by allowing time and/or dosage control of the treatment.

1. Conditional Modulators

In some embodiments, expression of any of the peptides or nucleic acids described herein may be externally controlled by treating the cell with a modulating factor, such as doxycycline, tetracycline or analogues thereof. Analogues of tetracycline are for example chlortetracycline, oxytetracycline, demethylchloro-tetracycline, methacycline, doxycycline and minocycline.

In some embodiments, reversible gene silencing may be implemented using a transactivator induced promoter together with said transactivator. In some embodiments, such a transactivator induced promoter comprises control elements for the enhancement or repression of transcription of the transgene or nucleic acid of interest. Control elements include, without limitation, operators, enhancers and promoters. In some embodiments, a transactivator inducible promoter is transcriptionally active when bound to a transactivator, which in turn is activated under a specific set of conditions, for example, in the presence or in the absence of a particular combination of chemical signals, for example, by a modulating factor selected for example from the previous list.

The transactivator induced promoter may be any promoter herein mentioned which has been modified to incorporate transactivator binding sequences, such as several tet-operator sequences, for example 3, 4, 5, 6, 7, 8, 9, or 10 tet-operator sequences. In some embodiments, the tet-operator sequences are in tandem. In some embodiments, the promoter is a tetracycline response element (TRE). Such sequences can for example replace the functional recognition sites for Staf and Oct-1 in the distal sequence element (DSE) of the U6 promoter, including the human U6 promoter.

Specific examples of transcription modulator domains that induce expression in the presence of modulating factor include, but are not limited to, the transcription modulator domains found in the following transcription modulators: the Tet-On transcription modulator; and the Tet-On Advanced transcription modulator and the Tet-On 3G transcription modulator; all of which are available from Clontech Laboratories, Mountain View, Calif. Specific examples of transcription modulator domains that induce expression in the absence of modulating factor include, but are not limited to, the transcription modulator domains found in the following transcription modulators: the Tet-off transcription modulator and the Tet-Off Advanced transcription modulator, both of which are available from Clontech Laboratories, Mountain View, Calif. These systems can be adapted and used according to procedures that are well known in the art and that will be familiar to the ordinarily skilled artisan.

In some embodiments, the transactivator induced promoter comprises a plurality of transactivator binding sequences operatively linked to the inhibitory nucleic acid molecule.

The transactivator may be provided by a nucleic acid sequence, in the same expression vector or in a different expression vector, comprising a modulating factor-dependent promoter operatively linked to a sequence encoding the transactivator. The term “different expression vector” is intended to include any vehicle for delivery of a nucleic acid, for example, a virus, plasmid, cosmid or transposon. Suitable promoters for use in said nucleic acid sequence include, for example, constitutive, regulated, tissue-specific or ubiquitous promoters, which may be of cellular, viral or synthetic origin, such as CMV, RSV, PGK, EF1α, NSE, synapsin, β-actin, GFAP.

An exemplary transactivator according to some embodiments is the rtTA-Oct2 transactivator composed of the DNA binding domain of rtTA2-M2 and of the Oct-2Q(Q→A) activation domain. Another exemplary transactivator according to some embodiments is the rtTA-Oct3 transactivator composed of the DNA binding domain of the Tet-repressor protein (E. coli) and of the Oct-2Q(Q→A) activation domain. Both are described in patent application WO 2007/004062.

Some embodiments include an isolated nucleotide sequence encoding a regulatory fusion protein (RPR), wherein the fusion protein contains (1) a transcription blocking domain capable of inhibiting expression of the nucleotide sequence of interest, and (2) a ligand-binding domain, wherein in the presence of a cognate ligand capable of binding the ligand-binding domain, the fusion protein is stabilized.

In some embodiments, the transcription blocking domain may be derived from a bacterial, bacteriophage, eukaryotic, or yeast repressor protein. In some embodiments, the transcription blocking domain is derived from a bacterial or bacteriophage repressor protein, such as, for example, TetR, LexA, LacI, TrpR, Arc, and LambdaCI. In some embodiments, the transcription blocking domain is derived from a eukaryotic repressor protein, such as, for example, GAL4. In some embodiments, the transcription blocking domain is a mutated restriction enzyme capable of binding but not cleaving DNA, and the operator is a recognition site for the restriction enzyme. In some embodiments, for example, the transcription blocking domain is a mutated NotI.

In some embodiments, the ligand-binding domain is derived from a steroid, thyroid, or retinoid receptor. In some embodiments, the ligand-binding domain is derived from an estrogen receptor, and the cognate ligand is an estrogen. In some embodiments, the estrogen receptor contains one or more mutations, for example, the T2 mutations, and the cognate ligand is tamoxifen. These systems can be adapted and used according to procedures that are well known in the art and that will be familiar to the ordinarily skilled artisan.

In some embodiments, the RheoSwitch system can be used to modulate transcription. In some embodiments, the RheoSwitch system includes a Rheoreceptor and Rheoactivator proteins, which can be activated by the presence of RSL1 ligand. In some embodiments, the receptor and activator stably dimerize and bind to the response element and turn on transcription in the presence of the RSL1 ligand (see, for example, the Instruction Manual for “RheoSwitch® Mammalian Inducible Expression System,” New England BioLabs® Inc., Version 1.3, November 2007; Karzenowski, D. et al., BioTechiques 39:191-196 (2005); Dai, X. et al., Protein Expr. Purif 42:236-245 (2005); Palli, S. R. et al., Eur. J. Biochem. 270:1308-1515 (2003); Dhadialla, T. S. et al., Annual Rev. Entomol. 43:545-569 (1998); Kumar, M. B, et al., J. Biol. Chem. 279:27211-27218 (2004); Verhaegent, M. and Christopoulos, T. K., Annal. Chem. 74:4378-4385 (2002); Katalam, A. K., et al., Molecular Therapy 13:S103 (2006); and Karzenowski, D. et al., Molecular Therapy 13:S194 (2006)).

In some embodiments, electromagnetic energy can be used to modulate transcription, including, for example, the systems and methods described in WO 2014/018423, incorporated herein by reference.

In some embodiments, controllable regulation of RNA transcription can be achieved by including a repressor binding region, such as, for example, from the lac repressor/operator system as modified for mammals. See Hu and Davidson, 1987, and Kozak, 1986.

2. Conditional Activity Via Site-Specific Recombination

In some embodiments, an introduced nucleic acid that is or encodes an inhibitory agent can be removed at a time subsequent to its integration in a host genome, such as by using site-specific recombination methods. In some embodiments, an inhibitory agent, such as a nucleic acid that is or encodes CRISPR, gRNA, Cas, ZFP, ZFN, TALE, TALEN, RNAi, siRNA, shRNA, miRNA, antisense RNA and/or ribozymes, is placed between recombination site sequences, such as loxP. In some embodiments, the nucleic acid includes at least one (typically two) site(s) for recombination mediated by a site-specific recombinase. In some embodiments, site-specific recombinases catalyze introduction or excision of DNA fragments from a longer DNA molecule. In some embodiments, these enzymes recognize a relatively short, unique nucleic acid sequence, which serves for both recognition and recombination. In some embodiments, a recombination site contains short inverted repeats (6, 7, or 8 base pairs in length) and the length of the DNA-binding element can be approximately 11 to approximately 13 bp in length.

In some embodiments, the vectors may comprise one or more recombination sites for any of a wide variety of site-specific recombinases. It is to be understood that the target site for a site-specific recombinase is in addition to any site(s) required for integration of a viral, e.g. lentiviral, genome. In some embodiments, a nucleic acid includes one or more sites for a recombinase enzyme selected from the group consisting of Cre, XerD, HP1 and Flp. These enzymes and their recombination sites are well known in the art (see, for example, Sauer et al., 1989, Nucleic Acids Res., 17:147; Gorman et al., 2000, Curr. Op. Biotechnol, 11:455; O'Gorman et al., 1991, Science, 251: 1351; Kolb, 2002, Cloning Stem Cells, 4:65; Kuhn et al., 2002, Methods MoI. Biol, 180:175).

In some embodiments, these recombinases catalyze a conservative DNA recombination event between two 34-bp recognition sites (loxP and FRT, respectively). In some embodiments, placing a heterologous nucleic acid sequence operably linked to a promoter element between two loxP sites (in which case the sequence is “floxed”) allows for controlled expression of the introduced nucleic acid encoding an inhibitory agent, such as any of those described herein, following transfer into a cell. By inducing expression of Cre within the cell, the heterologous nucleic acid sequence is excised, thus preventing further transcription and/or effectively eliminating expression of the sequence. Some embodiments comprise Cre-mediated gene activation, in which either heterologous or endogenous genes may be activated, e.g., by removal of an inhibitory element or a polyadenylation site.

As described above, positioning a heterologous nucleic acid sequence between loxP sites allows for controlled expression of the heterologous sequence following transfer into a cell. By inducing Cre expression within the cell, the heterologous nucleic acid sequence can be excised, thus preventing further transcription and/or effectively eliminating expression of the sequence. Cre expression may be induced in any of a variety of ways. For example, Cre may be present in the cells under control of an inducible promoter, and Cre expression may be induced by activating the promoter. Alternatively or additionally, Cre expression may be induced by introducing an expression vector that directs expression of Cre into the cell. Any suitable expression vector can be used, including, but not limited to, viral vectors such as lentiviral or adenoviral vectors. The phrase “inducing Cre expression” as used herein refers to any process that results in an increased level of Cre within a cell.

Lentiviral transfer plasmids comprising two loxP sites are useful in any applications for which standard vectors comprising two loxP sites can be used. For example, selectable markers may be placed between the loxP sites. This allows for sequential and repeated targeting of multiple genes to a single cell (or its progeny). After introduction of a transfer plasmid comprising a floxed selectable marker into a cell, stable transfectants may be selected. After isolation of a stable transfectant, the marker can be excised by induction of Cre. The marker may then be used to target a second gene to the cell or its progeny. Lentiviral particles comprising a lentiviral genome derived from the transfer plasmids may be used in the same manner.

In some embodiments, transfer plasmids and lentiviral particles may be used to achieve constitutive, conditional, reversible, or tissue-specific expression in cells, tissues, or organisms. Some embodiments include a method of reversibly expressing a transcript in a cell comprising: (i) delivering a lentiviral vector to the cell, wherein the lentiviral vector comprises a heterologous nucleic acid, and wherein the heterologous nucleic acid is located between sites for a site-specific recombinase; and (ii) inducing expression of the site-specific recombinase within the cell, thereby preventing synthesis of the transcript within those cells. According to some embodiments, a nucleic acid encoding the site-specific recombinase is operably linked to an inducible promoter, and the inducing step comprises inducing the promoter as described above.

E. Delivery of Agents, Nucleic Acids Encoding the Gene Disrupting Molecules and Complexes

In some aspects, a nucleic acid encoding a nucleic acid molecule that is, includes or encodes a nucleic acid inhibitory molecule, such as an RNA interfering molecule, DNA-targeting molecule, complex thereof (e.g. Cas9/gRNA RNPs), or combination, is administered or introduced to the cell. In some embodiments, such nucleic acid molecule or complex thereof can be introduced into cells, such as T cells, by methods well known in the art. Such methods include, but are not limited to, introduction in the form of recombinant viral vectors (e.g. retroviruses, lentiviruses, adenoviruses), liposomes or nanoparticles. In some embodiments, methods can include microinjection, electroporation, particle bombardment, Calcium Phosphate transfection, cell compression, squeezing. In some embodiments, the polynucleotides may be included in vectors, more particularly plasmids or virus, in view of being expressed in the cells.

In some embodiments, viral and non-viral based gene transfer methods can be used to introduce nucleic acids into cells, such as T cells. Such methods can be used to administer nucleic acids encoding components to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

In some embodiments, delivery is via the use of RNA or DNA viral based systems for the delivery of nucleic acids. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon. TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1:13-26 (1994). Viral-based systems in some embodiments include retroviral, lentivirus, adenoviral, adeno-associated and herpes simplex virus vectors for gene transfer.

In some embodiments, the nucleic acid is administered in the form of an expression vector, such as a viral expression vector. In some aspects, the expression vector is a retroviral expression vector, an adenoviral expression vector, a DNA plasmid expression vector, or an AAV expression vector. In some embodiments, the introduced vector, such as a viral vector, also includes nucleic acid encoding the genetically engineered antigen receptor, such as CAR. In some embodiments, the nucleic acids can be provided on separate expression cassettes operably linked to a promoter for control of separate expression therefrom.

In some aspects, a reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into the cell to encode a gene product which serves as a marker by which to measure the alteration or modification of expression of the gene product. In a further embodiment, the DNA molecule encoding the gene product may be introduced into the cell via a vector. In some embodiments, the gene product is luciferase. In a further embodiment, the expression of the gene product is decreased.

In some embodiments, an agent capable of inducing a genetic disruption, such as a knockdown or a knockout of genes encoding PD-1 and/or PD-L1, such as PDCD1 and/or CD274, is introduced as a complex, such as a ribonucleoprotein (RNP) complex. RNP complexes include a sequence of ribonucleotides, such as an RNA or a gRNA molecule, and a polypeptide, such as a Cas9 protein or variant thereof. In some embodiments, the Cas9 protein is delivered as an RNP complex that comprises a Cas9 protein and a gRNA molecule, e.g., a gRNA targeted for PDCD1 or CD274. In some embodiments, the RNP that includes one or more gRNA molecules targeted for PDCD1 or CD274, and a Cas9 enzyme or variant thereof, is directly introduced into the cell via physical delivery (e.g., electroporation, particle gun, Calcium Phosphate transfection, cell compression or squeezing), liposomes or nanoparticles. In particular embodiments, the RNP includes one or more gRNA molecules targeted for PDCD1 or CD274 and a Cas9 enzyme or variant thereof is introduced via electroporation.

In some embodiments, the degree of knockout of a gene (e.g., PDCD1 or CD274) at various time points, e.g., 24 to 72 hours after introduction of agent, can be assessed using any of a number of well-known assays for assessing gene disruption in cells. Degree of knockdown of a gene (e.g., PDCD1 or CD274) at various time points, e.g., 24 to 72 hours after introduction of agent, can be assessed using any of a number of well-known assays for assessing gene expression in cells, such as assays to determine the level of transcription or protein expression or cell surface expression.

### IV. Compositions, Formulations and Methods of Administration

Also provided are cells, cell populations, and compositions (including pharmaceutical and therapeutic compositions) containing the cells and populations, such as cells and populations produced by the provided methods. Also provided are methods, e.g., therapeutic methods for administrating the cells and compositions to subjects, e.g., patients.

A. Compositions and Formulations

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions may be administered using standard administration techniques, formulations, and/or devices. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

B. Methods of Administration and Uses of Cells in Adoptive Cell Therapy

Provided are methods of administering the cells, populations, and compositions, and uses of such cells, populations, and compositions to treat or prevent diseases, conditions, and disorders, including cancers. In some embodiments, the cells, populations, and compositions are administered to a subject or patient having the particular disease or condition to be treated, e.g., via adoptive cell therapy, such as adoptive T cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in a cancer expressing an antigen recognized by an engineered T cell.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

As used herein, a “subject” is a mammal, such as a human or other animal, and typically is human. In some embodiments, the subject, e.g., patient, to whom the cells, cell populations, or compositions are administered is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

In some embodiments, the cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse.

In some aspects, the subject has not received prior treatment with another therapeutic agent.

Among the diseases, conditions, and disorders for treatment with the provided compositions, cells, methods and uses are tumors, including solid tumors, hematologic malignancies, and melanomas, and infectious diseases, such as infection with a virus or other pathogen, e.g., HIV, HCV, HBV, CMV, and parasitic disease. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), acute-lymphoblastic leukemia (ALL), non-Hodgkin's lymphoma, acute myeloid leukemia, multiple myeloma, refractory follicular lymphoma, mantle cell lymphoma, indolent B cell lymphoma, B cell malignancies, cancers of the colon, lung, liver, breast, prostate, ovarian, skin, melanoma, bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, and/or mesothelioma.

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease multiple sclerosis, asthma, and/or a disease or condition associated with transplant.

In some embodiments, the antigen associated with the disease or disorder is selected from the group consisting of orphan tyrosine kinase receptor ROR1, tEGFR, Her2, L1-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3 and/or biotinylated molecules, and/or molecules expressed by HIV, HCV, HBV or other pathogens.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4+ to CD8+ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8+ and CD4+ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4+ to CD8+ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4+ to CD8+ cells, and/or is based on a desired fixed or minimum dose of CD4+ and/or CD8+ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 104 and at or about 109 cells/kilograms (kg) body weight, such as between 105 and 106 cells/kg body weight, for example, at least or at least about or at or about 1×105 cells/kg, 1.5×105 cells/kg, 2×105 cells/kg, or 1×106 cells/kg body weight. For example, in some embodiments, the cells are administered at, or within a certain range of error of, between at or about 104 and at or about 109 T cells/kilograms (kg) body weight, such as between 105 and 106 T cells/kg body weight, for example, at least or at least about or at or about 1×105 T cells/kg, 1.5×105 T cells/kg, 2×105 T cells/kg, or 1×106 T cells/kg body weight.

In some embodiments, the cells are administered at or within a certain range of error of between at or about 104 and at or about 109 CD4+ and/or CD8+ cells/kilograms (kg) body weight, such as between 105 and 106 CD4+ and/or CD8+ cells/kg body weight, for example, at least or at least about or at or about 1×105 CD4+ and/or CD8+ cells/kg, 1.5×105 CD4+ and/or CD8+ cells/kg, 2×105 CD4+ and/or CD8+ cells/kg, or 1×106 CD4+ and/or CD8+ cells/kg body weight.

In some embodiments, the cells are administered at or within a certain range of error of, greater than, and/or at least about 1×106, about 2.5×106, about 5×106, about 7.5×106, or about 9×106 CD4+ cells, and/or at least about 1×106, about 2.5×106, about 5×106, about 7.5×106, or about 9×106 CD8+ cells, and/or at least about 1×106, about 2.5×106, about 5×106, about 7.5×106, or about 9×106 T cells. In some embodiments, the cells are administered at or within a certain range of error of between about 108 and 1012 or between about 1010 and 1011 T cells, between about 108 and 1012 or between about 1010 and 1011 CD4+ cells, and/or between about 108 and 1012 or between about 1010 and 1011 CD8+ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4+ to CD8+ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

In certain embodiments, the engineered cells are further modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 111 (1995), and U.S. Pat. No. 5,087,616.

Dosing Schedule or Regimen

In some embodiments, repeated dosage methods are provided in which a first dose of cells is given followed by one or more second consecutive doses. The timing and size of the multiple doses of cells generally are designed to increase the efficacy and/or activity and/or function of antigen-expressing T cells, such as CAR-expressing T cells, when administered to a subject in adoptive therapy methods. In some embodiments, the repeated dosings reduce the downregulation or inhibiting activity that can occur when inhibitory immune molecules, such as PD-1 and/or PD-L1 are upregulated on antigen-expressing, such as CAR-expressing, T cells. The methods involve administering a first dose, generally followed by one or more consecutive doses, with particular time frames between the different doses.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the first or consecutive dose is a single or continuous administration of the specified number of cells, given or initiated at a single point in time. In some contexts, however, the first or consecutive dose is administered in multiple injections or infusions over a period of no more than three days, such as once a day for three days or for two days or by multiple infusions over a single day period.

Thus, in some aspects, the cells of the first dose are administered in a single pharmaceutical composition. In some embodiments, the cells of the consecutive dose are administered in a single pharmaceutical composition.

In some embodiments, the cells of the first dose are administered in a plurality of compositions, collectively containing the cells of the first dose. In some embodiments, the cells of the consecutive dose are administered in a plurality of compositions, collectively containing the cells of the consecutive dose. In some aspects, additional consecutive doses may be administered in a plurality of compositions over a period of no more than 3 days.

The term “split dose” refers to a dose that is split so that it is administered over more than one day. This type of dosing is encompassed by the present methods and is considered to be a single dose.

Thus, the first dose and/or consecutive dose(s) in some aspects may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments, 33% of the first dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

With reference to a prior dose, such as a first dose, the term “consecutive dose” refers to a dose that is administered to the same subject after the prior, e.g., first, dose without any intervening doses having been administered to the subject in the interim. Nonetheless, the term does not encompass the second, third, and/or so forth, injection or infusion in a series of infusions or injections comprised within a single split dose. Thus, unless otherwise specified, a second infusion within a one, two or three-day period is not considered to be a “consecutive” dose as used herein. Likewise, a second, third, and so-forth in the series of multiple doses within a split dose also is not considered to be an “intervening” dose in the context of the meaning of “consecutive” dose. Thus, unless otherwise specified, a dose administered a certain period of time, greater than three days, after the initiation of a first or prior dose, is considered to be a “consecutive” dose even if the subject received a second or subsequent injection or infusion of the cells following the initiation of the first dose, so long as the second or subsequent injection or infusion occurred within the three-day period following the initiation of the first or prior dose.

Thus, unless otherwise specified, multiple administrations of the same cells over a period of up to 3 days is considered to be a single dose, and administration of cells within 3 days of an initial administration is not considered a consecutive dose and is not considered to be an intervening dose for purposes of determining whether a second dose is “consecutive” to the first.

In some embodiments, multiple consecutive doses are given, in some aspects using the same timing guidelines as those with respect to the timing between the first dose and first consecutive dose, e.g., by administering a first and multiple consecutive doses, with each consecutive dose given within a period of time in which an inhibitory immune molecule, such as PD-1 and/or PD-L1, has been upregulated in cells in the subject from an administered first dose. It is within the level of a skilled artisan to empirically determine when to provide a consecutive dose, such as by assessing levels of PD-1 and/or PD-L1 in antigen-expressing, such as CAR-expressing cells, from peripheral blood or other bodily fluid.

In some embodiments, the timing between the first dose and first consecutive dose, or a first and multiple consecutive doses, is such that each consecutive dose is given within a period of time is greater than about 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days or more. In some embodiments, the consecutive dose is given within a time period that is less than about 28 days after the administration of the first or immediately prior dose. The additional multiple additional consecutive dose or doses also are referred to as subsequent dose or subsequent consecutive dose.

The size of the first and/or one or more consecutive doses of cells are generally designed to provide improved efficacy and/or reduced risk of toxicity. In some aspects, a dosage amount or size of a first dose or any consecutive dose is any dosage or amount as described above. In some embodiments, the number of cells in the first dose or in any consecutive dose is between about 0.5×106 cells/kg body weight of the subject and 5×106 cells/kg, between about 0.75×106 cells/kg and 3×106 cells/kg or between about 1×106 cells/kg and 2×106 cells/kg, each inclusive.

As used herein, “first dose” is used to describe the timing of a given dose being prior to the administration of a consecutive or subsequent dose. The term does not necessarily imply that the subject has never before received a dose of cell therapy or even that the subject has not before received a dose of the same cells or cells expressing the same recombinant receptor or targeting the same antigen.

In some embodiments, the receptor, e.g., the CAR, expressed by the cells in the consecutive dose contains at least one immunoreactive epitope as the receptor, e.g., the CAR, expressed by the cells of the first dose. In some aspects, the receptor, e.g., the CAR, expressed by the cells administered in the consecutive dose is identical to the receptor, e.g., the CAR, expressed by the first dose or is substantially identical to the receptor, e.g., the CAR, expressed by the cells of administered in the first dose.

The recombinant receptors, such as CARs, expressed by the cells administered to the subject in the various doses generally recognize or specifically bind to a molecule that is expressed in, associated with, and/or specific for the disease or condition or cells thereof being treated. Upon specific binding to the molecule, e.g., antigen, the receptor generally delivers an immunostimulatory signal, such as an ITAM-transduced signal, into the cell, thereby promoting an immune response targeted to the disease or condition. For example, in some embodiments, the cells in the first dose express a CAR that specifically binds to an antigen expressed by a cell or tissue of the disease or condition or associated with the disease or condition.

### V. Definitions

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, “percent (%) amino acid sequence identity” and “percent identity” when used with respect to an amino acid sequence (reference polypeptide sequence) is defined as the percentage of amino acid residues in a candidate sequence (e.g., a streptavidin mutein) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative amino acid substitutions will involve exchanging a member of one of these classes for another class.

As used herein, a subject includes any living organism, such as humans and other mammals. Mammals include, but are not limited to, humans, and non-human animals, including farm animals, sport animals, rodents and pets.

As used herein, a composition refers to any mixture of two or more products, substances, or compounds, including cells. It may be a solution, a suspension, liquid, powder, a paste, aqueous, non-aqueous or any combination thereof.

As used herein, “enriching” when referring to one or more particular cell type or cell population, refers to increasing the number or percentage of the cell type or population, e.g., compared to the total number of cells in or volume of the composition, or relative to other cell types, such as by positive selection based on markers expressed by the population or cell, or by negative selection based on a marker not present on the cell population or cell to be depleted. The term does not require complete removal of other cells, cell type, or populations from the composition and does not require that the cells so enriched be present at or even near 100% in the enriched composition.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control or fluorescence minus one (FMO) gating control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control or fluorescence minus one (FMO) gating control under otherwise identical conditions, and/or at a level substantially lower than that for cell known to be positive for the marker, and/or at a level substantially similar as compared to that for a cell known to be negative for the marker.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### VI. Exemplary Embodiments

Among the exemplary embodiments are:

1. An engineered T cell, comprising:

(a) a genetically engineered antigen receptor that specifically binds to an antigen; and

(b) an inhibitory nucleic acid molecule that reduces, or is capable of effecting reduction of, expression of PD-L1.

2. The cell of embodiment 1, wherein the inhibitory nucleic acid molecule comprises an RNA interfering agent.

3. The cell of embodiment 1 or embodiment 2, wherein the inhibitory nucleic acid is or comprises or encodes a small interfering RNA (siRNA), a microRNA-adapted shRNA, a short hairpin RNA (shRNA), a hairpin siRNA, a precursor microRNA (pre-miRNA) or a microRNA (miRNA).

4. The cell of any of embodiments 1-3, wherein the inhibitory nucleic acid molecule comprises a sequence complementary to a PD-L1-encoding nucleic acid.

5. The cell of embodiment 1, wherein the inhibitory nucleic acid molecule comprises an antisense oligonucleotide complementary to a PD-L1-encoding nucleic acid.

6. A genetically engineered T cell, comprising:

(a) a genetically engineered antigen receptor that specifically binds to an antigen; and

(b) a disrupted PD-L1 gene, an agent for disruption of a PD-L1 gene, and/or disruption of a gene encoding PD-L1.

7. The cell of embodiment 6, wherein disruption of the gene is mediated by a gene editing nuclease, a zinc finger nuclease (ZFN), a clustered regularly interspaced short palindromic nucleic acid (CRISPR)/Cas9, and/or a TAL-effector nuclease (TALEN).

8. The cell of embodiment 6 or embodiment 7, wherein the disruption comprises a deletion of at least a portion of at least one exon of the gene.

9. The cell of any of embodiments 6-8, wherein:

the disruption comprises a deletion, mutation, and/or insertion in the gene resulting in the presence of a premature stop codon in the gene; and/or

the disruption comprises a deletion, mutation, and/or insertion within a first or second exon of the gene.

10. The cell of any of embodiments 1-9, wherein expression of PD-L1 in the T cell is reduced by at least 50, 60, 70, 80, 90, or 95% as compared to the expression in the T cell in the absence of the inhibitory nucleic acid molecule or gene disruption or in the absence of activation thereof.

11. A genetically engineered T cell, comprising:

(a) a genetically engineered antigen receptor that specifically binds to an antigen; and

(b) a polynucleotide encoding a molecule that reduces or disrupts expression of PD-1 or PD-L1 in the cell, wherein expression or activity of the polynucleotide is conditional.

12. The cell of embodiment 11, wherein the expression is under the control of a conditional promoter or enhancer or transactivator.

13. The cell of embodiment 12, wherein the conditional promoter or enhancer or transactivator is an inducible promoter, enhancer, or transactivator or a repressible promoter, enhancer, or transactivator.

14. The genetically engineered T cell of embodiment 13, wherein the molecule that reduces or disrupts expression of PD-1 or PD-L1 is or comprises or encodes an antisense molecule, siRNA, shRNA, miRNA, a gene editing nuclease, zinc finger nuclease protein (ZFN), a TAL-effector nuclease (TALEN) or a CRISPR-Cas9 combination that specifically binds to, recognizes, or hybridizes to the gene.

15. The cell of any of embodiments 12-14, wherein the promoter is selected from among an RNA pol I, pol II or pol III promoter.

16. The cell of embodiment 15, wherein the promoter is selected from:

a pol III promoter that is a U6 or H1 promoter; or

a pol II promoter that is a CMV, SV40 early region or adenovirus major late promoter.

17. The cell of any of embodiments 12-16, wherein the promoter is an inducible promoter.

18. The cell of embodiment 17, wherein the promoter comprises a Lac operator sequence, a tetracycline operator sequence, a galactose operator sequence or a doxycycline operator sequence, or is an analog thereof.

19. The cell of any of embodiments 12-16, wherein the promoter is a repressible promoter.

20. The cell of embodiment 19, wherein the promoter comprises a Lac repressible element or a tetracycline repressible element, or is an analog thereof.

21. The cell of any of embodiments 1-20, wherein the T cell is a CD4+ or CD8+ T cell.

22. The cell of any of embodiments 1-21, wherein the genetically engineered antigen receptor is a functional non-T cell receptor.

23. The cell of any of embodiments 1-22, wherein the genetically engineered antigen receptor is a chimeric antigen receptor (CAR).

24. The cell of embodiment 23, wherein the CAR comprises an extracellular antigen-recognition domain that specifically binds to the antigen and an intracellular signaling domain comprising an ITAM.

25. The cell of embodiment 24, wherein the intracellular signaling domain comprises an intracellular domain of a CD3-zeta (CD3ζ) chain.

26. The cell of embodiment 24 or embodiment 25, wherein the CAR further comprises a costimulatory signaling region.

27. The cell of embodiment 26, wherein the costimulatory signaling region comprises a signaling domain of CD28 or 4-1BB.

28. The cell of embodiment 26 or embodiment 27, wherein the costimulatory domain is CD28.

29. The cell of any of embodiments 1-28 that is a human cell.

30. The cell of any of embodiments 1-29 that is an isolated cell.

31. A nucleic acid molecule, comprising a first nucleic acid, which is optionally a first expression cassette, encoding an antigen receptor (CAR) and a second nucleic acid, which is optionally a second expression cassette, encoding an inhibitory nucleic acid molecule against PD-1 or PD-L1.

32. The nucleic acid molecule of embodiment 31, wherein the inhibitory nucleic acid molecule comprises an RNA interfering agent.

33. The nucleic acid molecule of embodiment 31 or embodiment 32, wherein the inhibitory nucleic acid is or comprises or encodes a small interfering RNA (siRNA), a microRNA-adapted shRNA, a short hairpin RNA (shRNA), a hairpin siRNA, a precursor microRNA (pre-miRNA) or a microRNA (miRNA).

34. The nucleic acid molecule of any of embodiments 31-33, wherein the inhibitory nucleic acid comprises a sequence complementary to a PD-L1-encoding nucleic acid.

35. The nucleic acid molecule of embodiment 31, wherein the inhibitory nucleic acid molecule comprises an antisense oligonucleotide complementary to a PD-L1-encoding nucleic acid.

36. The nucleic acid molecule of any of embodiments 31-35, wherein the antigen receptor is a functional non-T cell receptor.

37. The nucleic acid molecule of any of embodiments 31-36, wherein the genetically engineered antigen receptor is a chimeric antigen receptor (CAR).

38. The nucleic acid molecule of embodiment 37, wherein the CAR comprises an extracellular antigen-recognition domain that specifically binds to the antigen and an intracellular signaling domain comprising an ITAM.

39. The nucleic acid molecule of embodiment 38, wherein the intracellular signaling domain comprises an intracellular domain of a CD3-zeta (CD3ζ) chain.

40. The nucleic acid molecule of embodiment 38 or embodiment 39, wherein the CAR further comprises a costimulatory signaling region.

41. The nucleic acid molecule of embodiment 40, wherein the costimulatory signaling region comprises a signaling domain of CD28 or 4-1BB.

42. The nucleic acid molecule of embodiment 40 or embodiment 41, wherein the costimulatory domain is CD28.

43. The nucleic acid molecule of any of embodiments 31-42, wherein the first and second nucleic acids, optionally the first and second expression cassettes, are operably linked to the same or different promoters.

44. The nucleic acid molecule of any of embodiments 31-43, wherein the first nucleic acid, optionally first expression cassette, is operably linked to an inducible promoter or a repressible promoter and the second nucleic acid, optionally second expression cassette, is operably linked to a constitutive promoter.

45. The nucleic acid molecule of any of embodiments 31-44 that is isolated.

46. A vector, comprising the nucleic acid molecule of any of embodiments 31-45.

47. The vector of embodiment 46, wherein the vector is a plasmid, lentiviral vector, retroviral vector, adenoviral vector, or adeno-associated viral vector.

48. The vector of embodiment 47 that is integrase defective.

49. A T cell, comprising the nucleic acid molecule of any of embodiments 31-45 or vector of any of embodiments 46-48.

50. The T cell of embodiment 49 that is a CD4+ or CD8+ T cell.

51. The T cell of embodiment 49 or embodiment 50 that is a human cell.

52. The T cell of any of embodiments 49-51 that is isolated.

53. A pharmaceutical composition, comprising the cell of any of embodiments 1-30 or 49-52 and a pharmaceutically acceptable carrier.

54. A method of producing a genetically engineered T cell, comprising:

(a) introducing a genetically engineered antigen receptor that specifically binds to an antigen into a population of cells comprising T cells; and

(b) introducing into the population of cells an agent capable of leading to a reduction of expression of PD-L1 and/or inhibiting upregulation of PD-L1 in T cells in the population upon incubation under one or more conditions, as compared to PD-L1 expression and/or upregulation in T cells in a corresponding population of cells not introduced with the agent upon incubation under the one or more conditions,

wherein steps (a) and (b) are carried out simultaneously or sequentially in any order, thereby introducing the genetically engineered antigen receptor and the agent into a T cell in the population.

55. A method of regulating expression of PD-L1 in a genetically engineered T cell, comprising introducing into a T cell an agent capable of leading to a reduction of expression of PD-L1 and/or inhibiting upregulation of PD-L1 in the cell upon incubation under one or more conditions, as compared to expression or upregulation of PD-L1 in a corresponding T cell not introduced with the agent upon incubation under the one or more conditions, said T cell comprising a genetically engineered antigen receptor that specifically binds to an antigen.

56. The method of embodiment 54 or embodiment 55, wherein incubation under conditions comprising the presence of antigen induces expression or upregulation of PD-L1 in the corresponding population comprising T cells not introduced with the agent.

57. The method of embodiment 56, wherein the incubation in the presence of antigen comprises incubating the cells in vitro with the antigen.

58. The method of embodiment 57, wherein the incubation in the presence of antigen is for 2 hours to 48 hours, 6 hours to 30 hours or 12 hours to 24 hours, each inclusive, or is for less than 48 hours, less than 36 hours or less than 24 hours.

59. The method of embodiment 56, wherein the incubation comprises administration of the cells to a subject under conditions whereby the engineered antigen receptor specifically binds to the antigen for at least a portion of the incubation.

60. The method of embodiment 59, wherein the incubation induces expression or upregulation within a period of 24 hours, 2 days, 3 days, 4 days, 5 days, 6 days, 7 days, 8 days, 9 days or 10 days following administration of cells to the subject.

61. The method of any of embodiments 54-60, wherein the reduction in expression or inhibition of upregulation of PD-L1 is by at least or at least about 30%, 40%, 50%, 60%, 70%, 80%, 90%, 95% or more.

62. The method of any of embodiments 54-61 that is performed ex vivo.

63. The method of any of embodiments 54-62, wherein the introducing in (b) is carried out by introducing a nucleic acid comprising a sequence encoding the agent.

64. The method of any of embodiments 54-63, wherein the introducing comprises inducing transient expression of the agent in the T cell to effect temporary reduction or disruption of expression of PD-L1 in the cell, and/or wherein the reduction or disruption is not permanent.

65. The method of any of embodiments 54-64, wherein expression or activity of the agent is conditional.

66. The method of embodiment 65, wherein the expression is under the control of a conditional promoter or enhancer or transactivator.

67. The method of embodiment 66, wherein the conditional promoter or enhancer or transactivator is an inducible promoter, enhancer or transactivator or a repressible promoter, enhancer or transactivator.

68. The method of embodiment 66 or embodiment 67, wherein the promoter is selected from an RNA pol I, pol II or pol III promoter.

69. The method of embodiment 68, wherein the promoter is selected from:

a pol III promoter that is a U6 or an H1 promoter; or

a pol II promoter that is a CMV, a SV40 early region or an adenovirus major late promoter.

70. The method of any of embodiments 66-69, wherein the promoter is an inducible promoter.

71. The method of embodiment 70, wherein the promoter comprises a Lac operator sequence, a tetracycline operator sequence, a galactose operator sequence or a doxycycline operator sequence.

72. The method of any of embodiments 66-69, wherein the promoter is a repressible promoter.

73. The method of embodiment 72, wherein the promoter comprises a Lac repressible element or a tetracycline repressible element.

74. The method of any of embodiments 54-63, wherein the agent is stably expressed in the T cell to effect continued reduction or disruption of expression of PD-L1 in the cell.

75. The method of any of embodiments 54-74, wherein the agent is a nucleic acid molecule that is contained in a viral vector.

76. The method of embodiment 75, wherein the viral vector is an adenovirus, lentivirus, retrovirus, herpesvirus or adeno-associated virus vector.

77. The method of any of embodiments 54-76, wherein the agent is an inhibitory nucleic acid molecule that reduces expression of PD-L1 in the cell.

78. The method of embodiment 77, wherein the inhibitory nucleic acid molecule comprises an RNA interfering agent.

79. The method of embodiment 77 or embodiment 78, wherein the inhibitory nucleic acid is or comprises or encodes a small interfering RNA (siRNA), a microRNA-adapted shRNA, a short hairpin RNA (shRNA), a hairpin siRNA, a precursor microRNA (pre-miRNA) or a microRNA (miRNA).

80. The method of any of embodiment 78 or embodiment 79, wherein the inhibitory nucleic acid molecule comprises a sequence complementary to a PD-L1-encoding nucleic acid.

81. The method of embodiment 77, wherein the inhibitory nucleic acid molecule comprises an antisense oligonucleotide complementary to a PD-L1-encoding nucleic acid.

82. The method of any of embodiments 54-81, wherein the effecting reduction and/or inhibiting upregulation comprises disrupting a gene encoding PD-L1.

83. The method of embodiment 82, wherein:

the disruption comprises disrupting the gene at the DNA level and/or

the disruption is not reversible; and/or

the disruption is not transient.

84. The method of embodiment 82 or 83, wherein the disruption comprises introducing in step (b) a DNA binding protein or DNA-binding nucleic acid that specifically binds to or hybridizes to the gene.

85. The method of embodiment 84, wherein the disruption comprises introducing: (i) a fusion protein comprising a DNA-targeting protein and a nuclease or (ii) an RNA-guided nuclease.

86. The method of embodiment 85, wherein the DNA-targeting protein or RNA-guided nuclease comprises a zinc finger protein (ZFP), a TAL protein, or a Cas protein guided by a clustered regularly interspaced short palindromic nucleic acid (CRISPR) specific for the gene.

87. The method of any of embodiments 82-86, wherein the disruption comprises introducing a zinc finger nuclease (ZFN), a TAL-effector nuclease (TALEN), or and a CRISPR-Cas9 combination that specifically binds to, recognizes, or hybridizes to the gene.

88. The method of any of embodiments 84-87, wherein the introducing is carried out by introducing a nucleic acid comprising a sequence encoding the DNA-binding protein, DNA-binding nucleic acid, and/or complex comprising the DNA-binding protein or DNA-binding nucleic acid.

89. The method of embodiment 88, wherein the nucleic acid is in a viral vector.

90. The method of any of embodiments 84-89, wherein the specific binding to the gene is within an exon of the gene and/or is within a portion of the gene encoding an N-terminus of the target antigen.

91. The method of any of embodiments 84-90, wherein the introduction thereby effects a frameshift mutation in the gene and/or an insertion of an early stop codon within the coding region of the gene.

92. The method of any of embodiments 54-91, further comprising (c) introducing into the cell an agent capable of leading to a reduction of expression of PD-1 and/or inhibiting upregulation of PD-1 in the cell upon incubation under the one or more conditions compared to PD-1 expression or upregulation in a corresponding cell not introduced with the agent upon incubation under the one or more conditions, wherein the reduction of expression and/or inhibition of upregulation is temporary or transient.

93. The method of embodiment 92, wherein the agent is inducibly expressed or repressed in the cell to effect conditional reduction or disruption of expression of PD-1 in the cell.

94. A method of producing a genetically engineered T cell, comprising:

(a) introducing a genetically engineered antigen receptor that specifically binds to an antigen into a population of cells comprising T cells; and

(b) introducing into the population of cells an agent capable of transient reduction of expression of PD-1 and/or a transient inhibition of upregulation of PD-1 in T cells in the population upon incubation under one or more conditions, as compared to PD-1 expression and/or upregulation in T cells in a corresponding population of cells not introduced with the agent upon incubation under the one or more conditions,

wherein steps (a) and (b) are carried out simultaneously or sequentially in any order, thereby introducing the genetically engineered antigen receptor and the agent into a T cell in the population.

95. A method of regulating expression of PD-1 in a genetically engineered T cell, comprising introducing into a T cell an agent capable of transient reduction of expression of PD-1 and/or a transient inhibition of upregulation of PD-1 in the cell upon incubation under one or more conditions, as compared to expression or upregulation of PD-1 in a corresponding T cell not introduced with the agent upon incubation under the one or more conditions, said T cell comprising an antigen receptor that specifically binds to an antigen.

96. The method of embodiment 94 or embodiment 95, wherein transient reduction comprises reversible reduction in expression of PD-1 in the cell.

97. The method of any of embodiments 94-96, wherein incubation under conditions comprising the presence of antigen induces expression or upregulation of PD-1 in the corresponding population comprising T cells not introduced with the agent.

98. The method of embodiment 97, wherein the incubation in the presence of antigen comprises incubating the cells in vitro with the antigen.

99. The method of embodiment 98, wherein the incubation in the presence of antigen is for 2 hours to 48 hours, 6 hours to 30 hours or 12 hours to 24 hours, each inclusive, or is for less than 48 hours, less than 36 hours or less than 24 hours.

100. The method of embodiment 97, wherein the incubation comprises administration of the cells to a subject under conditions whereby the engineered antigen receptor specifically binds to the antigen for at least a portion of the incubation.

101. The method of embodiment 100, wherein the incubation induces expression or upregulation within a period of 24 hours, 2 days, 3 days, 4 days, 5 days, 6 days, 7 days, 8 days, 9 days or 10 days following administration of cells to the subject.

102. The method of any of embodiments 94-101, wherein the reduction in expression or inhibition of upregulation of PD-1 is by at least or at least about 30%, 40%, 50%, 60%, 70%, 80%, 90%, 95% or more.

103. The method of any of embodiments 94-102 that is performed ex vivo.

104. The method of any of embodiments 94-103, wherein the introducing in (b) is carried out by introducing into the cell a nucleic acid comprising a sequence encoding the agent.

105. The method of any of embodiments 94-104, wherein the agent is transiently expressed in the cell to effect temporary reduction or disruption of expression of PD-1 in the T cell.

106. The method of any of embodiments 94-105, wherein the expression or activity of the agent is conditional.

107. The method of embodiment 106, wherein the expression is under the control of a conditional promoter or enhancer or transactivator.

108. The method of embodiment 107, wherein the conditional promoter or enhancer or transactivator is an inducible promoter, enhancer or transactivator is a repressible promoter, enhancer or transactivator.

109. The method of embodiment 108, wherein the promoter is selected from an RNA pol I, pol II or pol III promoter.

110. The method of embodiment 109, wherein the promoter is selected from:

a pol III promoter that is a U6 or an H1 promoter; or

a pol II promoter that is a CMV, a SV40 early region or an adenovirus major late promoter.

111. The method of any of embodiments 108-110, wherein the promoter is an inducible promoter.

112. The method of embodiment 111, wherein the promoter comprises a Lac operator sequence, a tetracycline operator sequence, a galactose operator sequence or a doxycycline operator sequence.

113. The method of any of embodiments 108-112, wherein the promoter is a repressible promoter.

114. The method of embodiment 113, wherein the promoter comprises a Lac repressible element or a tetracycline repressible element.

115. The method of any of embodiments 92-114, wherein the agent is an inhibitory nucleic acid molecule that reduces expression of PD-1 in the cell.

116. The method of embodiment 115, wherein the inhibitory nucleic acid molecule comprises an RNA interfering agent.

117. The method of embodiment 115 or embodiment 116, wherein the inhibitory nucleic acid is or comprises or encodes a small interfering RNA (siRNA), a microRNA-adapted shRNA, a short hairpin RNA (shRNA), a hairpin siRNA, a precursor microRNA (pre-miRNA) or a microRNA (miRNA).

118. The method of any of embodiments 115-117, wherein the inhibitory nucleic acid molecule comprises a sequence complementary to a PD-L1-encoding nucleic acid.

119. The method of embodiment 115, wherein the inhibitory nucleic acid molecule comprises an antisense oligonucleotide complementary to a PD-L1-encoding nucleic acid.

120. The method of any of embodiments 54-119, wherein the T cell is a CD4+ or CD8+ T cell.

121. The method of any of embodiments 54-120, wherein the genetically engineered antigen receptor is a functional non-T cell receptor.

122. The method of any of embodiments 54-121, wherein the genetically engineered antigen receptor is a chimeric antigen receptor (CAR).

123. The method of embodiment 122, wherein the CAR comprises an extracellular antigen-recognition domain that specifically binds to the antigen and an intracellular signaling domain comprising an ITAM.

124. The method of embodiment 123, wherein the intracellular signaling domain comprises an intracellular domain of a CD3-zeta (CD3ζ) chain.

125. The method of embodiment 123 or embodiment 124, wherein the CAR further comprises a costimulatory signaling region.

126. The method of embodiment 125, wherein the costimulatory signaling region comprises a signaling domain of CD28 or 4-1BB.

127. The method of embodiment 125 or embodiment 126, wherein the costimulatory domain is CD28.

128. The method of embodiment 127, wherein the steps (a) and (b) are performed simultaneously, said steps comprising introducing a nucleic acid molecule comprising a first nucleic acid, which is optionally a first expression cassette, encoding the antigen receptor and a second nucleic acid, which is optionally a second expression cassette, encoding the agent to effect reduction of expression of PD-1 or PD-L1.

129. The method of embodiment 127 or embodiment 128, further comprising introducing into the population of cells a second genetically engineered antigen receptor that specifically binds to the same or a different antigen, said second antigen receptor comprising a co-stimulatory molecule other than CD28.

130. A method of producing a genetically engineered T cell, comprising:

(a) introducing a first genetically engineered antigen receptor that specifically binds to a first antigen into a population of cells comprising T cells, said first antigen receptor comprising a CD28 co-stimulatory molecule;

(b) introducing into the population of cells comprising T cells a second genetically engineered antigen receptor that specifically binds to the same or different antigen; and

(c) introducing into the population of cells comprising T cells an agent capable of leading to a reduction of expression of PD-1 or PD-L1 and/or inhibiting upregulation of PD-1 or PD-L1 in T cells in the population upon incubation under one or more conditions, as compared to PD-1 and/or PD-L1 expression or upregulation in T cells in a corresponding population of cells not introduced with the agent upon incubation under the one or more conditions, thereby introducing the first antigen receptor, the second antigen receptor and the agent into a T cell in the population.

131. The method of embodiment 130, wherein incubation under conditions comprising the presence of antigen induces expression or upregulation of PD-1 and/or PD-L1 in the corresponding population comprising T cells not introduced with the agent.

132. The method of embodiment 131, wherein the incubation in the presence of antigen comprises incubating the cells in vitro with the antigen.

133. The method of embodiment 132, wherein the incubation in the presence of antigen is for 2 hours to 48 hours, 6 hours to 30 hours or 12 hours to 24 hours, each inclusive, or is for less than 48 hours, less than 36 hours or less than 24 hours.

134. The method of embodiment 131, wherein the incubation comprises administration of the cells to a subject under conditions whereby the engineered antigen receptor specifically binds to the antigen for at least a portion of the incubation.

135. The method of embodiment 134, wherein the incubation induces expression or upregulation within a period of 24 hours, 2 days, 3 days, 4 days, 5 days, 6 days, 7 days, 8 days, 9 days or 10 days following administration of cells to the subject.

136. The method of any of embodiments 130-135, wherein expression or upregulation of PD-1 and/or PD-L1 in the cells in inhibited or reduced by at least or at least about 30%, 40%, 50%, 60%, 70%, 80%, 90%, 95% or more compared to an engineered cell produced by the method in the absence of introducing the agent.

137. The method of any of embodiments 129-136, wherein the first and second genetically engineered antigen receptor bind the same antigen.

138. The method of any of embodiments 130-137, wherein the second antigen receptor comprises a co-stimulatory molecule other than CD28.

139. The method of any of embodiments 129-138, wherein the costimulatory molecule other than CD28 is 4-1BB.

140. The method of any of embodiments 130-139, wherein the agent effects reduction of expression and/or inhibition of upregulation of PD-L1.

141. The method of any of embodiments 130-140, wherein steps (a) and (b) are performed simultaneously, said steps comprising introducing a nucleic acid molecule comprising a first nucleic acid, which is optionally a first expression cassette, encoding the antigen receptor and a second nucleic acid, which is optionally a second expression cassette, encoding the agent to effect reduction of expression of PD-1 or PD-L1.

142. The method of embodiment 141, wherein the first and second nucleic acids, optionally the first and second expression cassettes, are operably linked to the same or different promoters.

143. The method of embodiment 141 or embodiment 142, wherein the first nucleic acid, optionally first expression cassette, is operably linked to an inducible promoter or a repressible promoter and the second nucleic acid, optionally second expression cassette, is operably linked to a constitutive promoter.

144. The method of any of embodiments 54-143 that is a human cell. 145. A method of producing a genetically engineered T cell, comprising:

(a) obtaining a population of primary cells comprising T cells;

(b) enriching for cells in the population that do not express a target antigen; and

(c) introducing into the population of cells a genetically engineered antigen receptor that specifically binds to the target antigen; thereby producing a genetically engineered T cell.

146. The method of embodiment 145, further comprising culturing and/or incubating the cells under stimulating conditions to effect proliferation of the cells, wherein the proliferation and/or expansion of cells is greater than in cells produced in the method but in the absence of enriching for cells that do not express the target antigen.

147. The method of embodiment 146, wherein proliferation and/or expansion of cells is at least or at least about 1.5-fold, 2-fold, 3-fold, 4-fold, 5-fold, 6-fold, 7-fold, 8-fold, 9-fold, 10-fold or more greater.

148. The method of any of embodiments 145-147, wherein enriching for cells that do not express a target antigen comprises negative selection to deplete cells expressing the target antigen or disruption of the gene encoding the target antigen in cells in the population.

149. The method of any of embodiments 146-148, wherein the stimulating condition comprises an agent capable of activating one or more intracellular signaling domains of one or more components of a TCR complex.

150. A cell produced by the method of any of embodiments 54-149.

151. A pharmaceutical composition, comprising the cell of embodiment 150 and a pharmaceutically acceptable carrier.

152. A method of treatment, comprising administering to a subject having a disease or condition the cell of any of embodiments 1-30, 49-52 or 150 or the pharmaceutical composition of embodiment 46 or 115.

153. The method of treatment of embodiment 152, wherein the cells are administered in a dosage regime comprising:

(a) administering to the subject a first dose of cells expressing a chimeric antigen receptor (CAR); and

(b) administering to the subject a consecutive dose of CAR-expressing cells, said consecutive dose being administered to the subject at a time when expression of PD-L1 is induced or upregulated on the surface of the CAR-expressing cells administered to the subject in (a) and/or said consecutive dose being administered to the subject at least 5 days after initiation of the administration in (a).

154. A method of treatment, comprising:

(a) administering to the subject a first dose of cells expressing a chimeric antigen receptor (CAR); and

(b) administering to the subject a consecutive dose of CAR-expressing cells said consecutive dose being administered to the subject at a time when expression of PD-L1 is induced or upregulated on the surface of the CAR-expressing cells administered to the subject in (a) and/or said consecutive dose being administered to the subject at least 5 days after initiation of the administration in (a).

155. The method of embodiment 153 or embodiment 154, wherein the consecutive dose of cells is administered at least or more than about 5 days after and less than about 12 days after initiation of said administration in (a)

156. The method of any of embodiments 153-155, wherein the number of cells administered in the first and/or second dose is between about 0.5×10⁶ cells/kg body weight of the subject and 4×10⁶ cells/kg, between about 0.75×10⁶ cells/kg and 3.0×10⁶ cells/kg or between about 1×10⁶ cells/kg and 2×10⁶ cells/kg, each inclusive.

157. The method of any of embodiments 152-156, wherein the genetically engineered antigen receptor specifically binds to an antigen associated with the disease or condition.

158. The method of treatment of any of embodiments 152-157, wherein the disease or condition is a cancer.

159. The method of any of embodiments 152-158, wherein the disease or condition is a leukemia or lymphoma.

160. The method of any of embodiments 152-159, wherein the disease or condition is acute lymphoblastic leukemia.

161. The method of any of embodiments 152-159, wherein the disease or condition is a non-Hodgkin lymphoma (NHL).

### VII. Examples

The following examples are included for illustrative purposes only and are not intended to limit the scope of the invention.

### Example 1: Assessment of PD-1/PD-L1 Expression in T-Cells Stimulated Through a Chimeric Antigen Receptor (CAR)

T cells were isolated by immunoaffinity-based enrichment from leukapheresis samples from human subjects, and cells were activated and transduced with a viral vector encoding an anti-CD19 chimeric antigen receptor (CAR) containing a human CD28-derived intracellular signaling domain and a human CD3 zeta-derived signaling domain. Surface expression on the resulting isolated compositions (of the CAR and of certain T cell markers) was assessed by flow cytometry, to determine, in the composition, the percentage of CAR+ cells among all T cells in the and among T cell subsets, as well as ratio of CD4+ to CD8+ T cells (see

TABLE 1
Anti-CD19 CAR Expression on Transduced T cells

| | CD3+CAR+ | CD4+CAR+ | CD8+CAR+ | CD3+CD4+ | CD3+CD8+ |
|---|---|---|---|---|---|
| percent | 49.91 | 23.60 | 28.73 | 40.03 | 53.66 |
| (average) | | | | | |
| Standard | 2.97 | 1.18 | 2.38 | 1.10 | 1.22 |
| Deviation | | | | | |

The composition then was subdivided into different samples by incubation with: 1) K562 cells expressing the antigen for which the CAR was specific (K562-tCD19 cells) (antigen-specific coculture); 2) K562 cells expressing an unrelated antigen (K562-ROR1 cells) (non-specific coculture control); or 3) plate-bound anti-CD3 antibody and soluble anti-CD28 antibody (for stimulation via the TCR complex), initially using plate-bound anti-CD3 and soluble anti-CD28, and at day 3, where applicable, incubation with engineered cells. For (1) and (2), K562 (immortalized myelogenous leukemia line) cells, were engineered to express CD19 and ROR1, respectively, and incubated with the CAR-expressing T cells at a 1:1 ratio. For each of the conditions, CAR-expressing T cells were stimulated for 24 hours. An unstimulated sample (“media,” no K562 cells or stimulating antibodies) was used as an additional negative control.

After 24 hours in culture, flow cytometry was performed to assess surface expression of PD-1, PD-L1, PD-L2, T cell markers, and CAR (based on goat-anti-mouse (“GAM”) staining to detect the murine variable region portion of the CAR) on the on cells in each sample. Live, single cells with forward scatter and side scatter profiles matching lymphocytes were gated for analysis. Expression of PD-1, PD-L1 and PD-L2 was assessed on various gated populations of T cells (CD4+/CAR+, CD4+/CAR−, CD8+/CAR+, and CD8+/CAR−), with gates set based on the surface expression of various markers, and using values for the negative control (“media”) sample to determine appropriate gating.

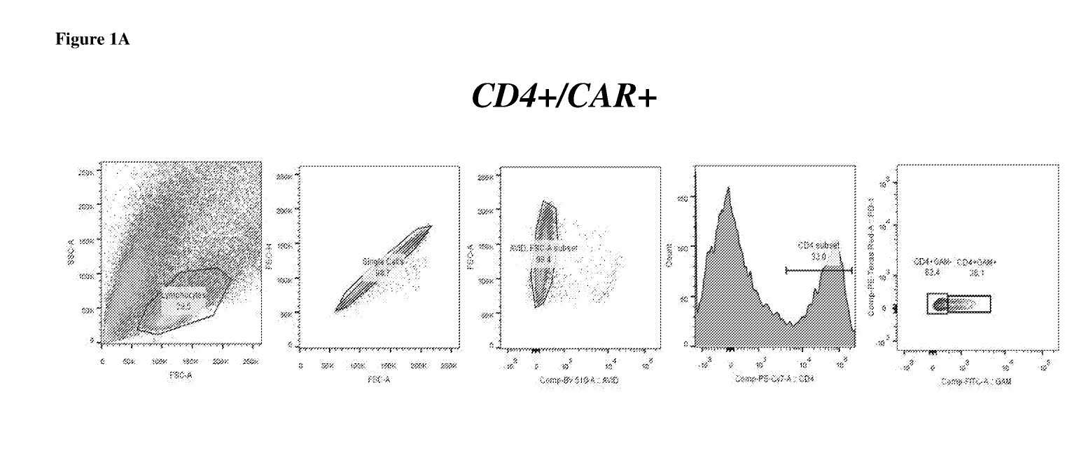

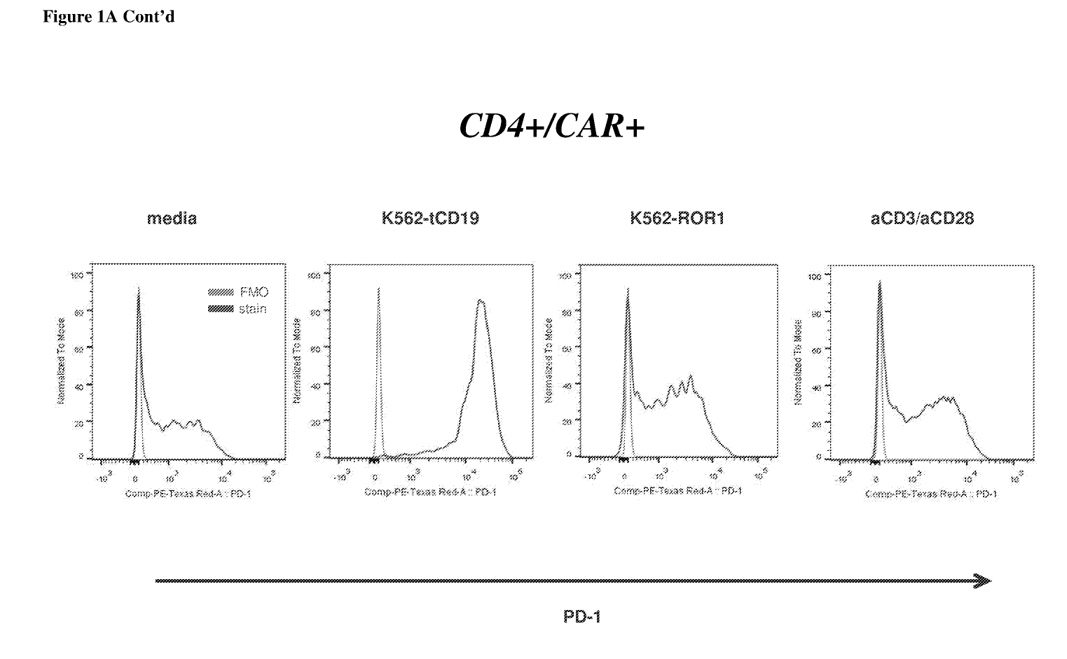

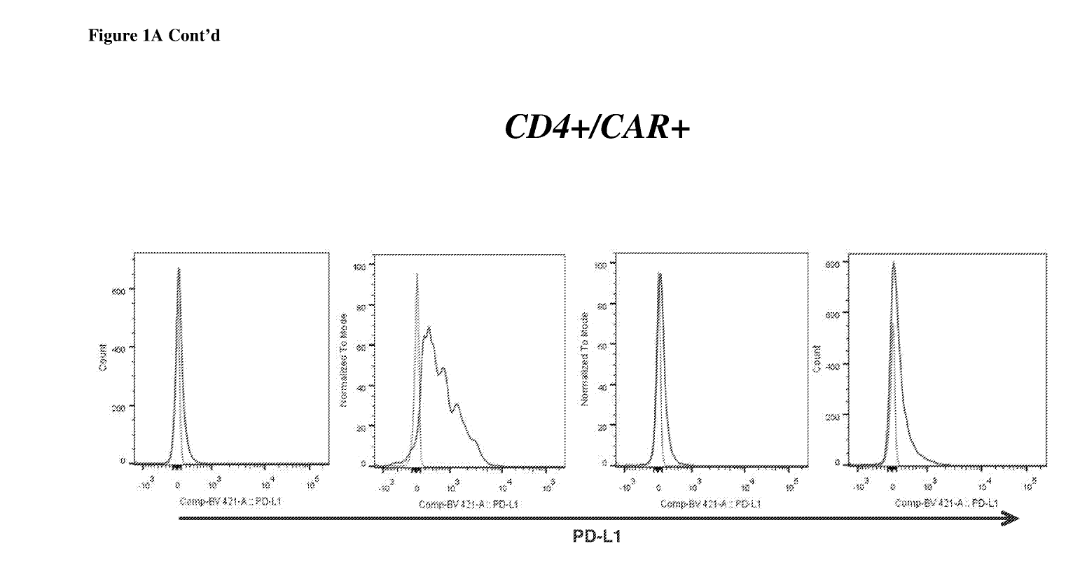

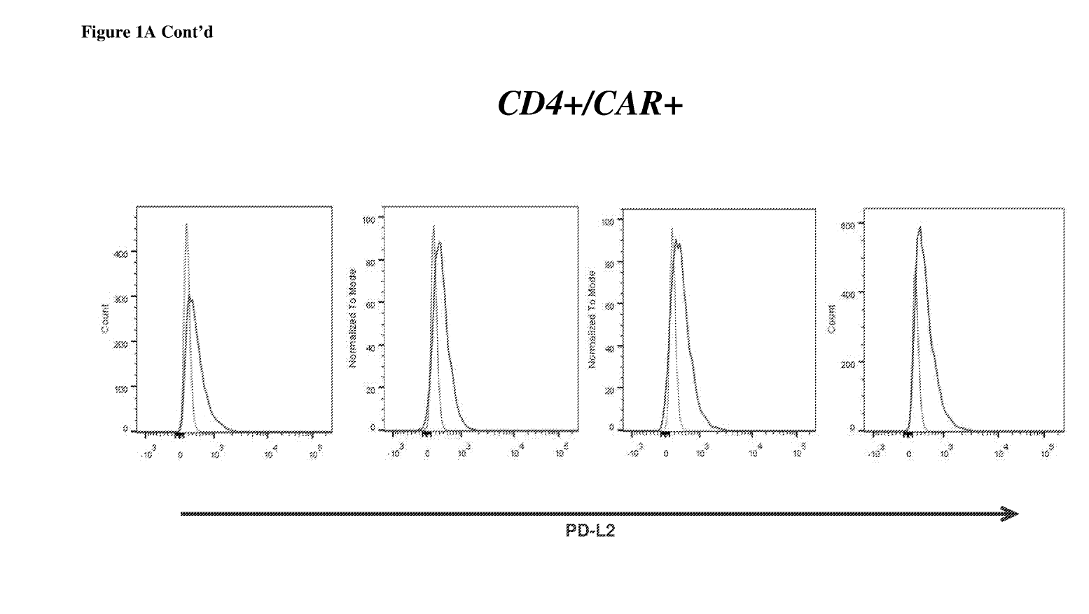

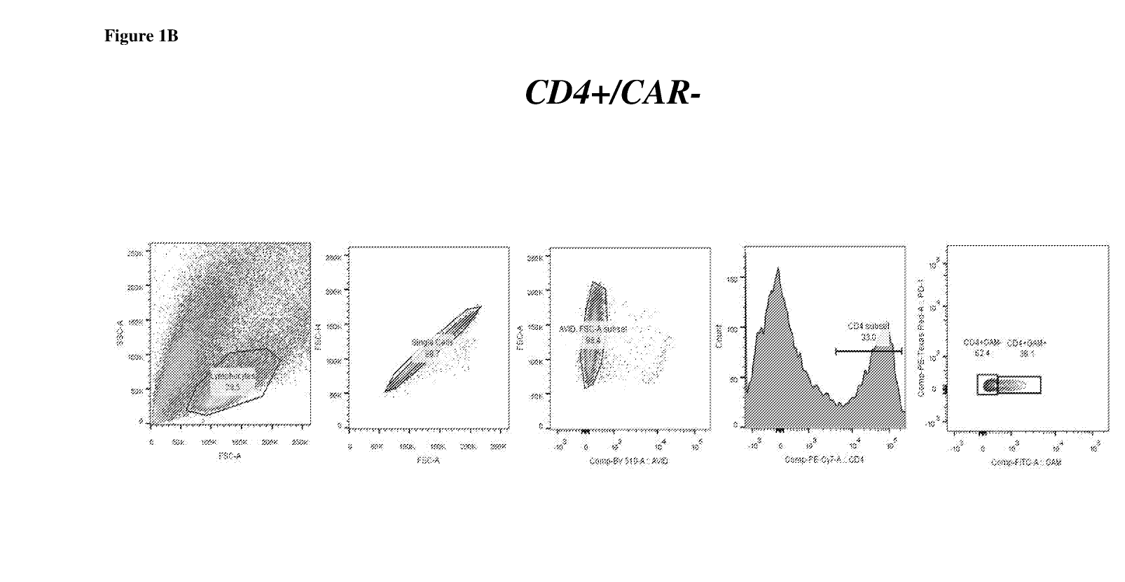

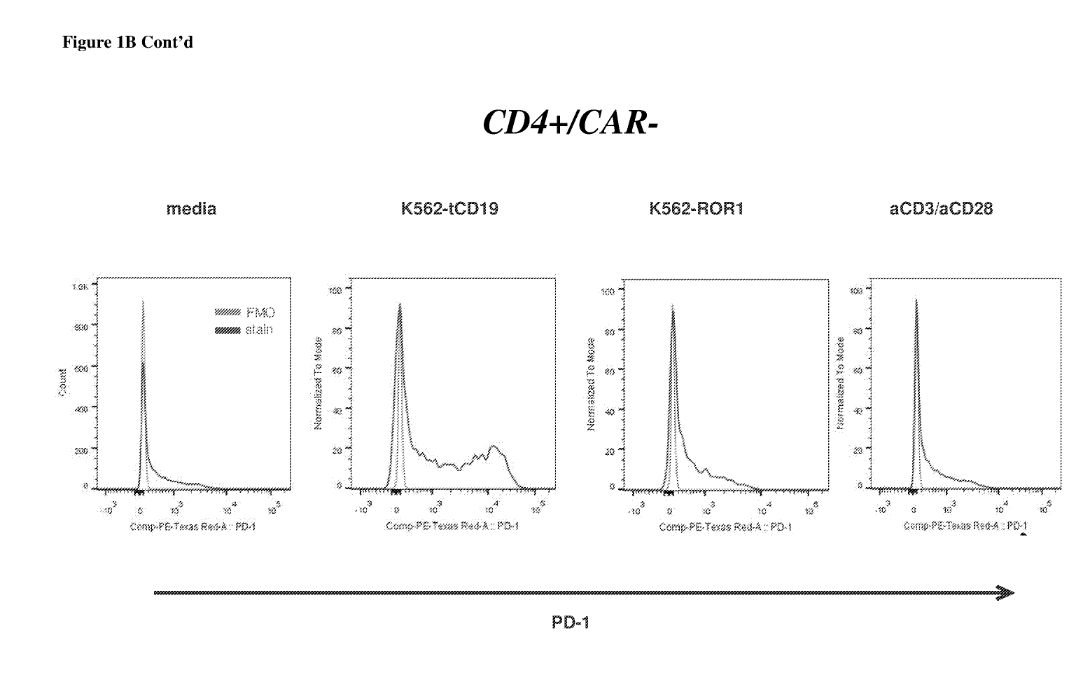

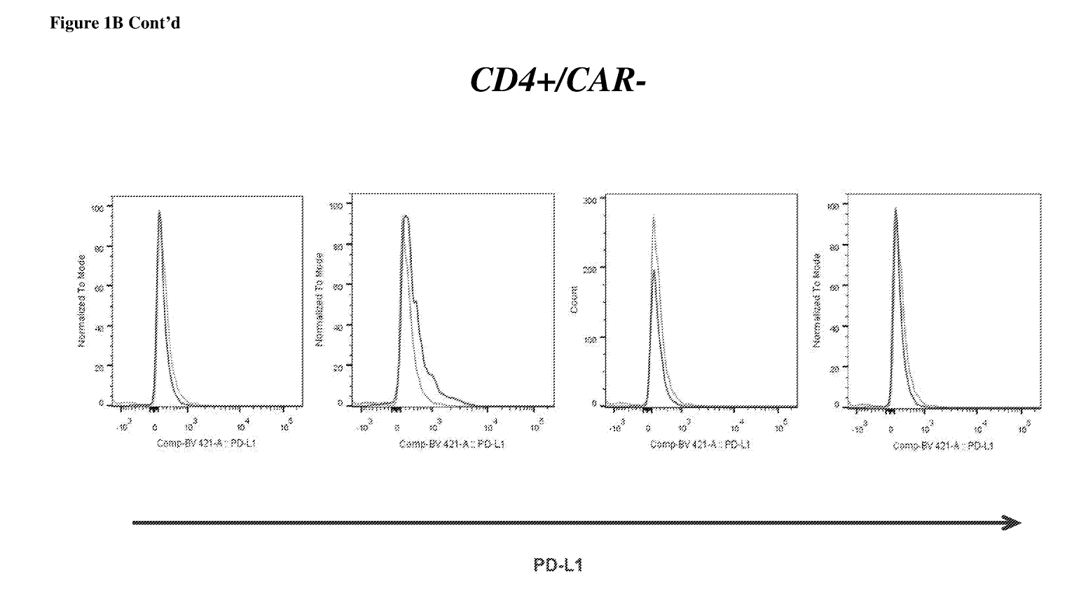

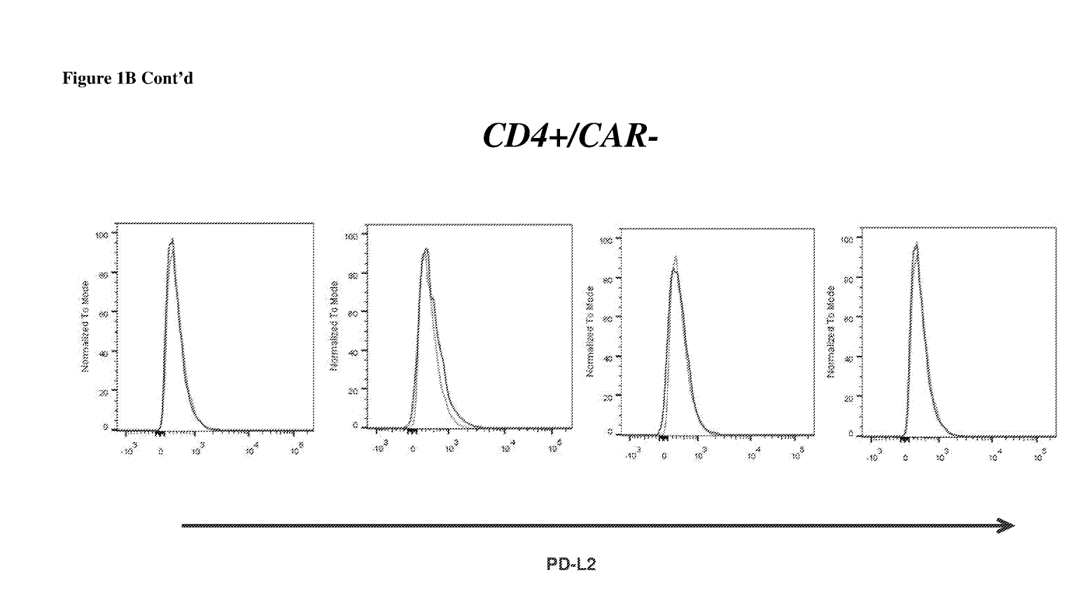

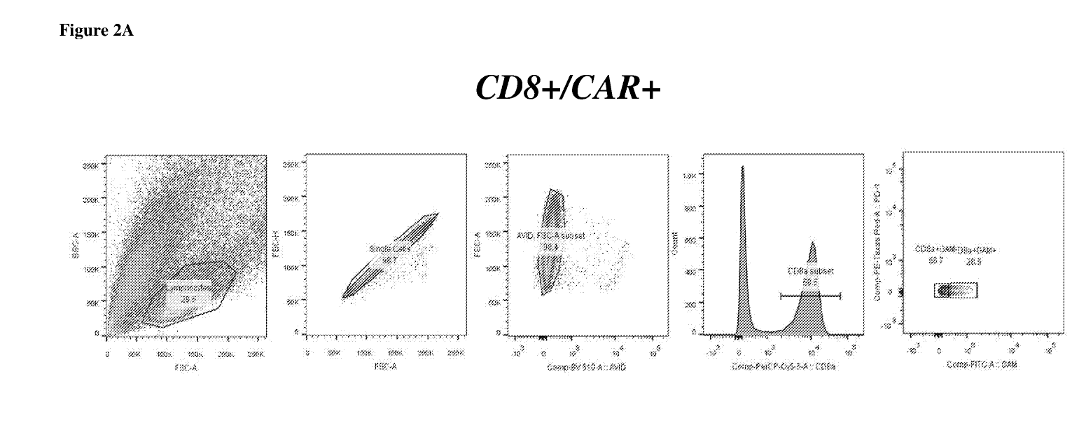

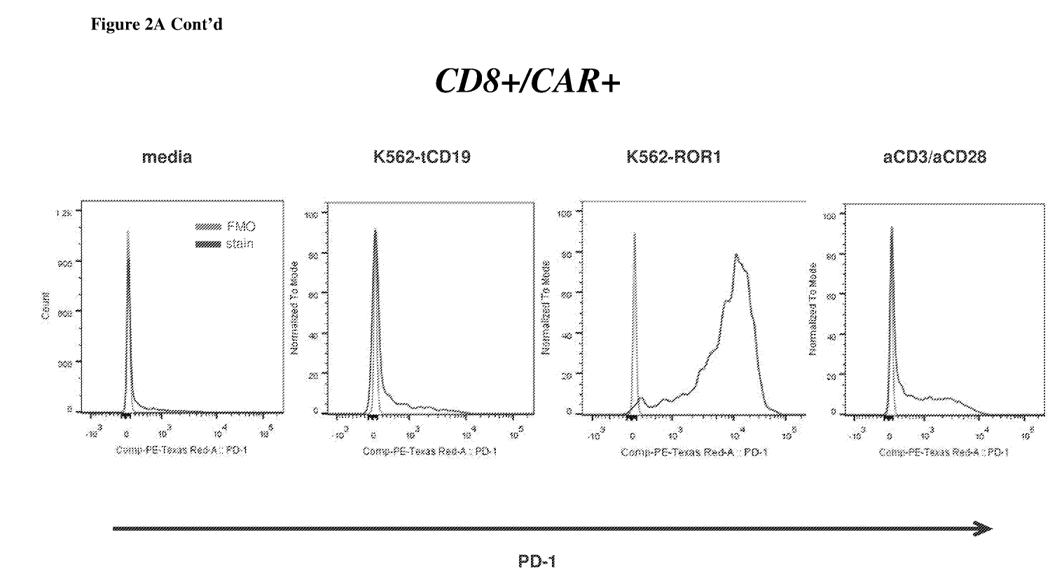

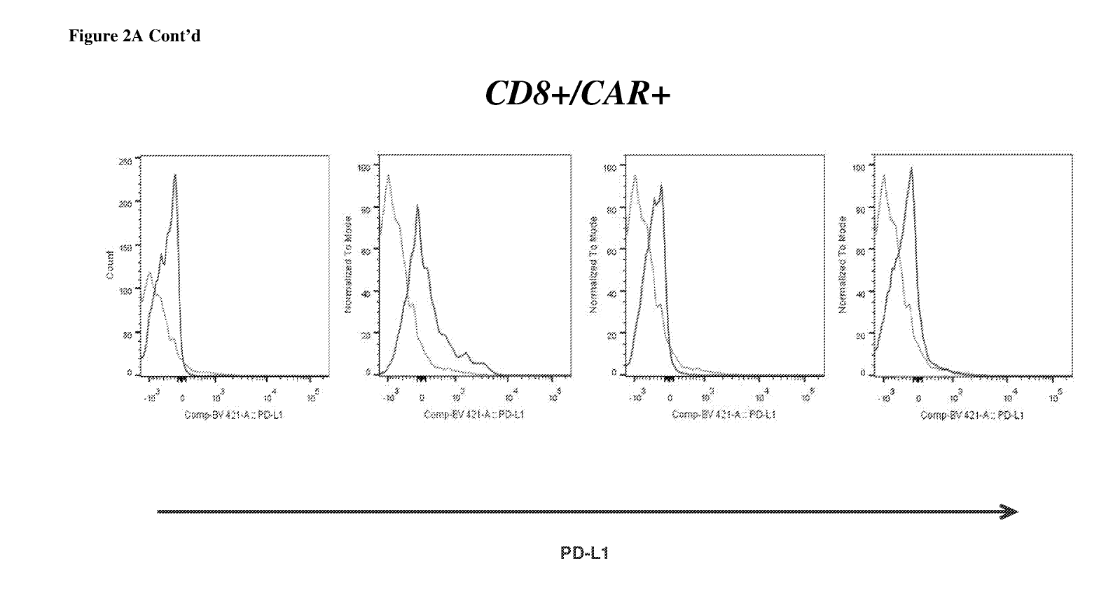

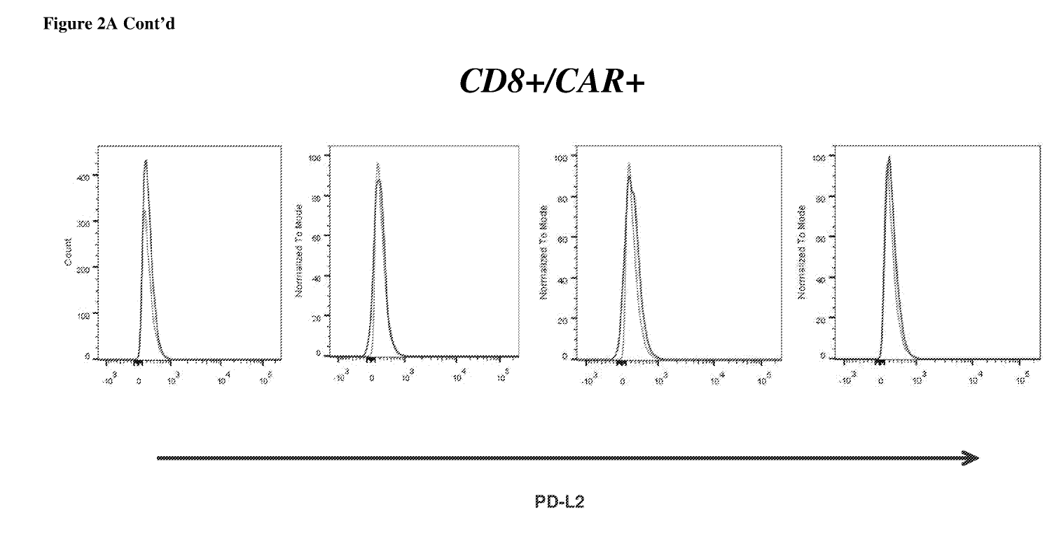

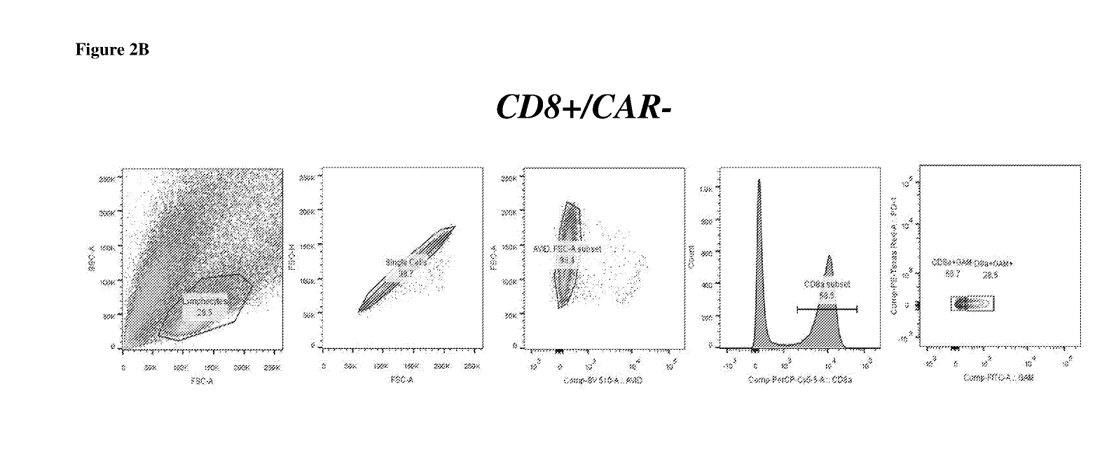

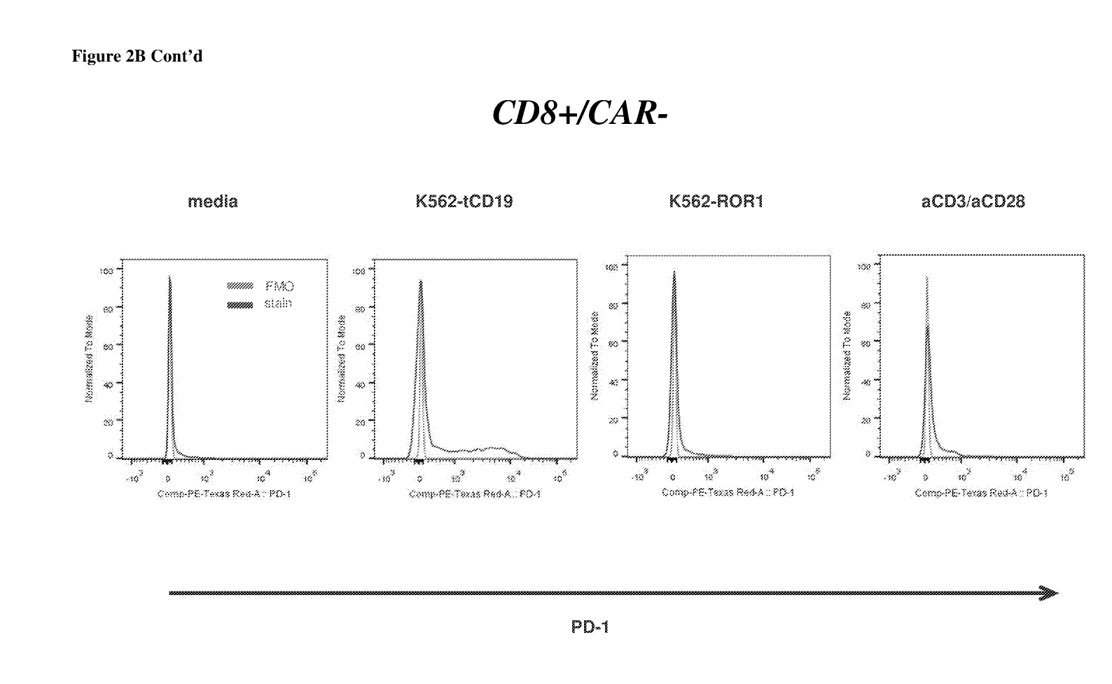

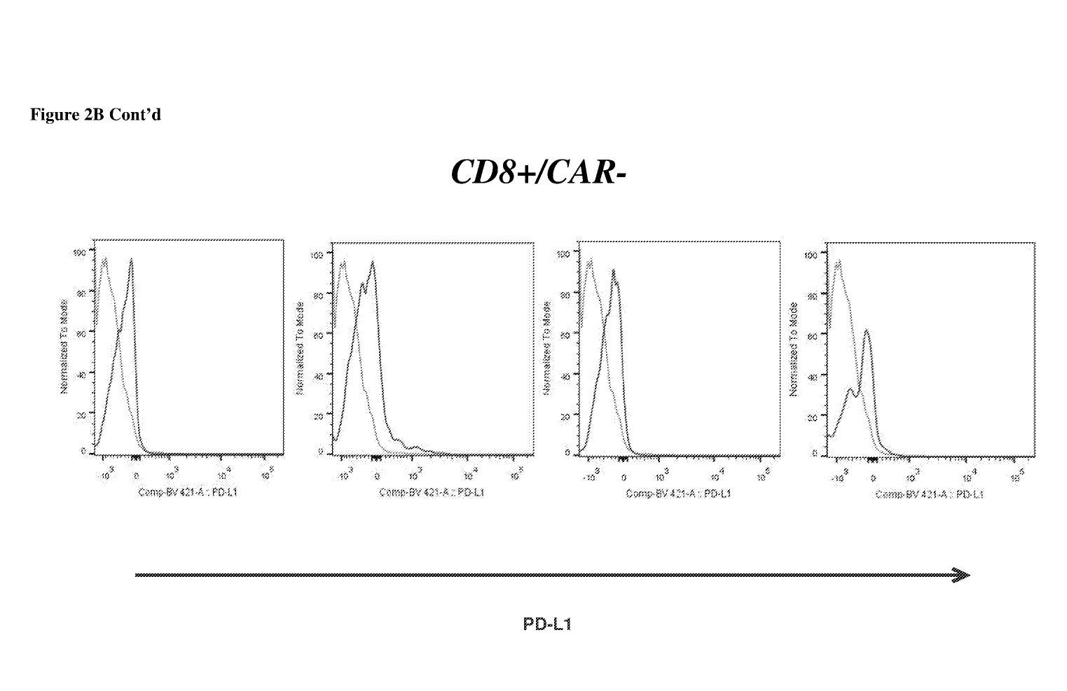

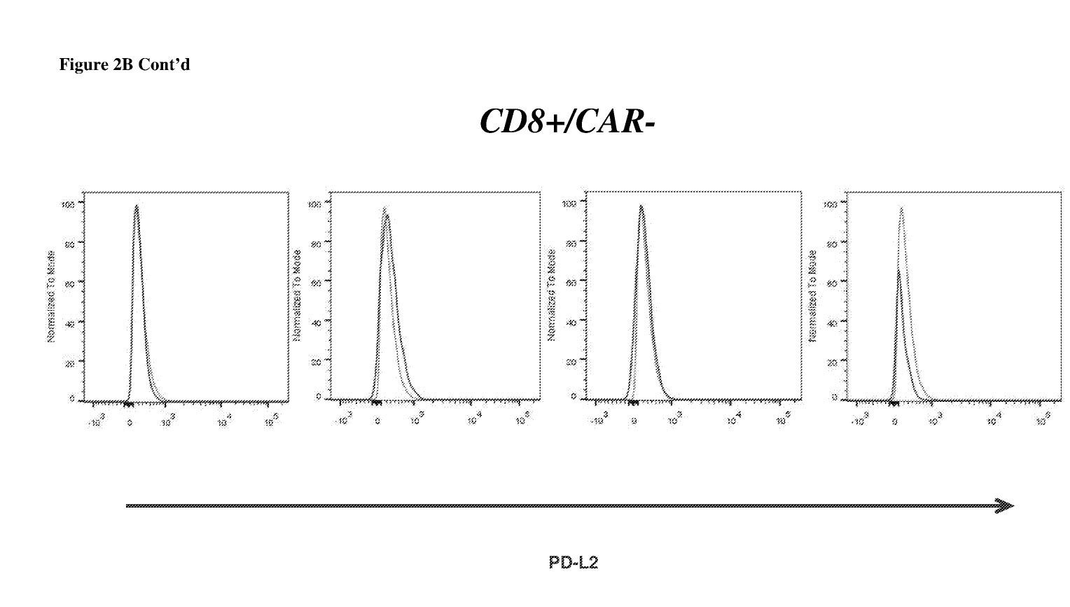

As shown in FIGS. 1A and 2A, PD-1 and PD-L1 expression increased within twenty-four (24) hours in both CD4+/CAR+ and CD8+/CAR+ T cells when cultured with cells expressing the antigen to which the CAR was specific (K562-tCD19). This increase in expression of PD-1 and PD-L1 was not observed within this timeframe in CAR+ cells incubated with cells of the same type expressing an irrelevant antigen (K562-ROR1) or in any of the CD4+ or CD8+ cell populations incubated under conditions designed to effect stimulation through the TCR complex (anti-CD3 and anti-CD28 antibodies). Expression of PD-L2 was not upregulated within this timeframe under any of the stimulated conditions tested.

As shown in FIGS. 1B and 2B, the increase in expression of PD-1 and PD-L1 in cells incubated with CD19-expressing cells was observed to be primarily due to expression of the anti-CD19 CAR. Neither the CD4+-gated nor the CD8+-gated T cells that did not express the CAR (“CAR−”) exhibited substantial increases in PD-1 or PD-L1 surface expression following incubation with the CD19-expressing cells.

Similar results were obtained in the presence of T cells genetically engineered with an anti-CD19 chimeric antigen receptor (CAR) containing a human 4-1BB-derived intracellular signaling domain and a human CD3 zeta-derived signaling domain. Thus, the results showed that the upregulation of PD-1 and PD-L1 occurred on T cells transduced with CAR constructs containing either a CD28 or 4-1BB costimulatory signaling domain. These data demonstrate upregulation in surface expression of PD-1 and PD-L1 within twenty-four hours following stimulation through the chimeric antigen receptor, but not following stimulation under conditions designed to mimic signal through the canonical T cell antigen receptor complex and associated costimulatory receptors (anti-CD3/anti-CD28 antibodies).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### SEQUENCES

| SEQ | | | |
|---|---|---|---|
| ID | | | |
| NO: | Type | Sequence | Description |
| 1 | RNA | aaugcguuca gcaaaugcca guagg | siRNA specific for PD- |
| | | | L1 |
| 2 | RNA | cuaauugucu auugggaaa | siRNA specific for PD- |
| | | | L1 |
| 3 | RNA | cgacuacaag cgaauuacu | siRNA specific for PD- |
| | | | L1 |
| 4 | RNA | CCUACUGGCAUUUGCUGAACGCAUU | siRNA specific for PD- |
| | | | L1 (sense sequence) |
| 5 | RNA | AAUGCGUUCAGCAAAUGCCAGUAGG | siRNA specific for PD- |
| | | | L1 (anti-sense |
| | | | sequence) |
| 6 | RNA | uuacgucucc uccaaaugug uauca | siRNA specific for PD- |
| | | | 1 |
| 7 | Protein | MRIFAVFIFMTYWHLLNAFTVTVPKDLYVVEYGSNMTIECKFPV | PD-L1 (Human) |
| | | EKQLDLAALIVYWEMEDKNIIQFVHGEEDLKVQHSSYRQRARLL | |
| | | KDQLSLGNAALQITDVKLQDAGVYRCMISYGGADYKRITVKVNA | |
| | | PYNKINQRILVVDPVTSEHELTCQAEGYPKAEVIWTSSDHQVLS | |
| | | GKTTTTNSKREEKLFNVTSTLRINTTTNEIFYCTFRRLDPEENH | |
| | | TAELVIPELPLAHPPNERTHLVILGAILLCLGVALTFIFRLRKG | |
| | | RMMDVKKCGIQDTNSKKQSDTHLEET | |
| 8 | DNA | ggcgcaacgc tgagcagctg gcgcgtcccg cgcggcccca | CD274 encoding PD- |
| | | gttctgcgca gcttcccgag gctccgcacc agccgcgctt | L1 (Human) |
| | | ctgtccgcct gcagggcatt ccagaaagat gaggatattt | |
| | | gctgtcttta tattcatgac ctactggcat ttgctgaacg | |
| | | catttactgt cacggttccc aaggacctat atgtggtaga | |
| | | gtatggtagc aatatgacaa ttgaatgcaa attcccagta | |
| | | gaaaaacaat tagacctggc tgcactaatt gtctattggg | |
| | | aaatggagga taagaacatt attcaatttg tgcatggaga | |
| | | ggaagacctg aaggttcagc atagtagcta cagacagagg | |
| | | gcccggctgt tgaaggacca gctctccctg ggaaatgctg | |
| | | cacttcagat cacagatgtg aaattgcagg atgcaggggt | |
| | | gtaccgctgc atgatcagct atggtggtgc cgactacaag | |
| | | cgaattactg tgaaagtcaa tgccccatac aacaaaatca | |
| | | accaaagaat tttggttgtg gatccagtca cctctgaaca | |
| | | tgaactgaca tgtcaggctg agggctaccc caaggccgaa | |
| | | gtcatctgga caagcagtga ccatcaagtc ctgagtggta | |
| | | agaccaccac caccaattcc aagagagagg agaagctttt | |
| | | caatgtgacc agcacactga gaatcaacac aacaactaat | |
| | | gagattttct actgcacttt taggagatta gatcctgagg | |
| | | aaaaccatac agctgaattg gtcatcccag aactacctct | |
| | | ggcacatcct ccaaatgaaa ggactcactt ggtaattctg | |
| | | ggagccatct tattatgcct tggtgtagca ctgacattca | |
| | | tcttccgttt aagaaaaggg agaatgatgg atgtgaaaaa | |
| | | atgtggcatc caagatacaa actcaaagaa gcaaagtgat | |
| | | acacatttgg aggagacgta atccagcatt ggaacttctg | |
| | | atcttcaagc agggattctc aacctgtggt ttaggggttc | |
| | | atcggggctg agcgtgacaa gaggaaggaa tgggcccgtg | |
| | | ggatgcaggc aatgtgggac ttaaaaggcc caagcactga | |
| | | aaatggaacc tggcgaaagc agaggaggag aatgaagaaa | |
| | | gatggagtca aacagggagc ctggagggag accttgatac | |
| | | tttcaaatgc ctgaggggct catcgacgcc tgtgacaggg | |
| | | agaaaggata cttctgaaca aggagcctcc aagcaaatca | |
| | | tccattgctc atcctaggaa gacgggttga gaatccctaa | |
| | | tttgagggtc agttcctgca gaagtgccct ttgcctccac | |
| | | tcaatgcctc aatttgtttt ctgcatgact gagagtctca | |
| | | gtgttggaac gggacagtat ttatgtatga gtttttccta | |
| | | tttattttga gtctgtgagg tcttcttgtc atgtgagtgt | |
| | | ggttgtgaat gatttctttt gaagatatat tgtagtagat | |
| | | gttacaattt tgtcgccaaa ctaaacttgc tgcttaatga | |
| | | tttgctcaca tctagtaaaa catggagtat ttgtaaggtg | |
| | | cttggtctcc tctataacta caagtataca ttggaagcat | |
| | | aaagatcaaa ccgttggttg cataggatgt cacctttatt | |
| | | taacccatta atactctggt tgacctaatc ttattctcag | |
| | | acctcaagtg tctgtgcagt atctgttcca tttaaatatc | |
| | | agctttacaa ttatgtggta gcctacacac ataatctcat | |
| | | ttcatcgctg taaccaccct gttgtgataa ccactattat | |
| | | tttacccatc gtacagctga ggaagcaaac agattaagta | |
| | | acttgcccaa accagtaaat agcagacctc agactgccac | |
| | | ccactgtcct tttataatac aatttacagc tatattttac | |
| | | tttaagcaat tcttttattc aaaaaccatt tattaagtgc | |
| | | ccttgcaata tcaatcgctg tgccaggcat tgaatctaca | |
| | | gatgtgagca agacaaagta cctgtcctca aggagctcat | |
| | | agtataatga ggagattaac aagaaaatgt attattacaa | |
| | | tttagtccag tgtcatagca taaggatgat gcgaggggaa | |
| | | aacccgagca gtgttgccaa gaggaggaaa taggccaatg | |
| | | tggtctggga cggttggata tacttaaaca tcttaataat | |
| | | cagagtaatt ttcatttaca aagagaggtc ggtacttaaa | |
| | | ataaccctga aaaataacac tggaattcct tttctagcat | |
| | | tatatttatt cctgatttgc ctttgccata taatctaatg | |
| | | cttgtttata tagtgtctgg tattgtttaa cagttctgtc | |
| | | ttttctattt aaatgccact aaattttaaa ttcatacctt | |
| | | tccatgattc aaaattcaaa agatcccatg ggagatggtt | |
| | | ggaaaatctc cacttcatcc tccaagccat tcaagtttcc | |
| | | tttccagaag caactgctac tgcctttcat tcatatgttc | |
| | | ttctaaagat agtctacatt tggaaatgta tgttaaaagc | |
| | | acgtattttt aaaatttttt tcctaaatag taacacattg | |
| | | tatgtctgct gtgtactttg ctatttttat ttattttagt | |
| | | gtttcttata tagcagatgg aatgaatttg aagttcccag | |
| | | ggctgaggat ccatgccttc tttgtttcta agttatcttt | |
| | | cccatagctt ttcattatct ttcatatgat ccagtatatg | |
| | | ttaaatatgt cctacatata catttagaca accaccattt | |
| | | gttaagtatt tgctctagga cagagtttgg atttgtttat | |
| | | gtttgctcaa aaggagaccc atgggctctc cagggtgcac | |
| | | tgagtcaatc tagtcctaaa aagcaatctt attattaact | |
| | | ctgtatgaca gaatcatgtc tggaactttt gttttctgct | |
| | | ttctgtcaag tataaacttc actttgatgc tgtacttgca | |
| | | aaatcacatt ttctttctgg aaattccggc agtgtacctt | |
| | | gactgctagc taccctgtgc cagaaaagcc tcattcgttg | |
| | | tgcttgaacc cttgaatgcc accagctgtc atcactacac | |
| | | agccctccta agaggcttcc tggaggtttc gagattcaga | |
| | | tgccctggga gatcccagag tttcctttcc ctcttggcca | |
| | | tattctggtg tcaatgacaa ggagtacctt ggctttgcca | |
| | | catgtcaagg ctgaagaaac agtgtctcca acagagctcc | |
| | | ttgtgttatc tgtttgtaca tgtgcatttg tacagtaatt | |
| | | ggtgtgacag tgttctttgt gtgaattaca ggcaagaatt | |
| | | gtggctgagc aaggcacata gtctactcag tctattccta | |
| | | agtcctaact cctccttgtg gtgttggatt tgtaaggcac | |
| | | tttatccctt ttgtctcatg tttcatcgta aatggcatag | |
| | | gcagagatga tacctaattc tgcatttgat tgtcactttt | |
| | | tgtacctgca ttaatttaat aaaatattct tatttatttt | |
| | | gttacttggt acaccagcat gtccattttc ttgtttattt | |
| | | tgtgtttaat aaaatgttca gtttaacatc ccagtggaga | |
| | | aagttaaaaa a | |
| 9 | Protein | MQIPQAPWPV VWAVLQLGWR PGWFLDSPDR PWNPPTFSPA | PD-1 (Flunmn) |
| | | LLVVTEGDNA TFTCSFSNTS ESFVLNWYRM SPSNQTDKLA | |
| | | AFPEDRSQPG QDCRFRVTQL PNGRDFHMSV VRARRNDSGT | |
| | | YLCGAISLAP KAQIKESLRA ELRVTERRAE VPTAHPSPSP | |
| | | RPAGQFQTLV VGVVGGLLGS LVLLVWVLAV ICSRAARGTI | |
| | | GARRTGQPLK EDPSAVPVFS VDYGELDFQW REKTPEPPVP | |
| | | CVPEQTEYAT IVFPSGMGTS SPARRGSADG PRSAQPLRPE | |
| | | DGHCSWPL | |
| 10 | DNA | agtttccctt ccgctcacct ccgcctgagc agtggagaag | PDCD1 encodingPD-1 |
| | | gcggcactct ggtggggctg ctccaggcat gcagatccca | MUM* |
| | | caggcgccct ggccagtcgt ctgggcggtg ctacaactgg | |
| | | gctggcggcc aggatggttc ttagactccc cagacaggcc | |
| | | ctggaacccc cccaccttct ccccagccct gctcgtggtg | |
| | | accgaagggg acaacgccac cttcacctgc agcttctcca | |
| | | acacatcgga gagcttcgtg ctaaactggt accgcatgag | |
| | | ccccagcaac cagacggaca agctggccgc cttccccgag | |
| | | gaccgcagcc agcccggcca ggactgccgc ttccgtgtca | |
| | | cacaactgcc caacgggcgt gacttccaca tgagcgtggt | |
| | | cagggcccgg cgcaatgaca gcggcaccta cctctgtggg | |
| | | gccatctccc tggcccccaa ggcgcagatc aaagagagcc | |
| | | tgcgggcaga gctcagggtg acagagagaa gggcagaagt | |
| | | gcccacagcc caccccagcc cctcacccag gccagccggc | |
| | | cagttccaaa ccctggtggt tggtgtcgtg ggcggcctgc | |
| | | tgggcagcct ggtgctgcta gtctgggtcc tggccgtcat | |
| | | ctgctcccgg gccgcacgag ggacaatagg agccaggcgc | |
| | | accggccagc ccctgaagga ggacccctca gccgtgcctg | |
| | | tgttctctgt ggactatggg gagctggatt tccagtggcg | |
| | | agagaagacc ccggagcccc ccgtgccctg tgtccctgag | |
| | | cagacggagt atgccaccat tgtctttcct agcggaatgg | |
| | | gcacctcatc ccccgcccgc aggggctcag ctgacggccc | |
| | | tcggagtgcc cagccactga ggcctgagga tggacactgc | |
| | | tcttggcccc tctgaccggc ttccttggcc accagtgttc | |
| | | tgcagaccct ccaccatgag cccgggtcag cgcatttcct | |
| | | caggagaagc aggcagggtg caggccattg caggccgtcc | |
| | | aggggctgag ctgcctgggg gcgaccgggg ctccagcctg | |
| | | cacctgcacc aggcacagcc ccaccacagg actcatgtct | |
| | | caatgcccac agtgagccca ggcagcaggt gtcaccgtcc | |
| | | cctacaggga gggccagatg cagtcactgc ttcaggtcct | |
| | | gccagcacag agctgcctgc gtccagctcc ctgaatctct | |
| | | gctgctgctg ctgctgctgc tgctgctgcc tgcggcccgg | |
| | | ggctgaaggc gccgtggccc tgcctgacgc cccggagcct | |
| | | cctgcctgaa cttgggggct ggttggagat ggccttggag | |
| | | cagccaaggt gcccctggca gtggcatccc gaaacgccct | |
| | | ggacgcaggg cccaagactg ggcacaggag tgggaggtac | |
| | | atggggctgg ggactcccca ggagttatct gctccctgca | |
| | | ggcctagaga agtttcaggg aaggtcagaa gagctcctgg | |
| | | ctgtggtggg cagggcagga aacccctcca cctttacaca | |
| | | tgcccaggca gcacctcagg ccctttgtgg ggcagggaag | |
| | | ctgaggcagt aagcgggcag gcagagctgg aggcctttca | |
| | | ggcccagcca gcactctggc ctcctgccgc cgcattccac | |
| | | cccagcccct cacaccactc gggagaggga catcctacgg | |
| | | tcccaaggtc aggagggcag ggctggggtt gactcaggcc | |
| | | cctcccagct gtggccacct gggtgttggg agggcagaag | |
| | | tgcaggcacc tagggccccc catgtgccca ccctgggagc | |
| | | tctccttgga acccattcct gaaattattt aaaggggttg | |
| | | gccgggctcc caccagggcc tgggtgggaa ggtacaggcg | |
| | | ttcccccggg gcctagtacc cccgccgtgg cctatccact | |
| | | cctcacatcc acacactgca cccccactcc tggggcaggg | |
| | | ccaccagcat ccaggcggcc agcaggcacc tgagtggctg | |
| | | ggacaaggga tcccccttcc ctgtggttct attatattat | |
| | | aattataatt aaatatgaga gcatgctaag gaaaa | |
| 11 | Protein | MDKKYSIGLDIGTNSVGWAVITDEYKVPSKKFKVLGNTDRHSIK | S. Pyogenes Cas9 |
| | | KNLIGALLFDSGETAEATRLKRTARRRYTRRKNRICYLQEIFSN | Q99ZW2 |
| | | EMAKVDDSFFHRLEESFLVEEDKKHERHPIFGNIVDEVAYHEKY | |
| | | PTIYHLRKKLVDSTDKADLRLIYLALAHMIKFRGHFLIEGDLNP | |
| | | DNSDVDKLFIQLVQTYNQLFEENPINASGVDAKAILSARLSKSR | |
| | | RLENLIAQLPGEKKNGLFGNLIALSLGLTPNFKSNFDLAEDAKL | |
| | | QLSKDTYDDDLDNLLAQIGDQYADLFLAAKNLSDAILLSDILRV | |
| | | NTEITKAPLSASMIKRYDEHHQDLTLLKALVRQQLPEKYKEIFF | |
| | | DQSKNGYAGYIDGGASQEEFYKFIKPILEKMDGTEELLVKLNRE | |
| | | DLLRKQRTFDNGSIPHQIHLGELHAILRRQEDFYPFLKDNREKI | |
| | | EKILTFRIPYYVGPLARGNSRFAWMTRKSEETITPWNFEEVVDK | |
| | | GASAQSFIERMTNFDKNLPNEKVLPKHSLLYEYFTVYNELTKVK | |
| | | YVTEGMRKPAFLSGEQKKAIVDLLFKTNRKVTVKQLKEDYFKKI | |
| | | ECFDSVEISGVEDRFNASLGTYHDLLKIIKDKDFLDNEENEDIL | |
| | | EDIVLTLTLFEDREMIEERLKTYAHLFDDKVMKQLKRRRYTGWG | |
| | | RLSRKLINGIRDKQSGKTILDFLKSDGFANRNFMQLIHDDSLTF | |
| | | KEDIQKAQVSGQGDSLHEHIANLAGSPAIKKGILQTVKVVDELV | |
| | | KVMGRHKPENIVIEMARENQTTQKGQKNSRERMKRIEEGIKELG | |
| | | SQILKEHPVENTQLQNEKLYLYYLQNGRDMYVDQELDINRLSDY | |
| | | DVDHIVPQSFLKDDSIDNKVLTRSDKNRGKSDNVPSEEVVKKMK | |
| | | NYWRQLLNAKLITQRKFDNLTKAERGGLSELDKAGFIKRQLVET | |
| | | RQITKHVAQILDSRMNTKYDENDKLIREVKVITLKSKLVSDFRK | |
| | | DFQFYKVREINNYHHAHDAYLNAVVGTALIKKYPKLESEFVYGD | |
| | | YKVYDVRKMIAKSEQEIGKATAKYFFYSNIMNFFKTEITLANGE | |
| | | IRKRPLIETNGETGEIVWDKGRDFATVRKVLSMPQVNIVKKTEV | |
| | | QTGGFSKESILPKRNSDKLIARKKDWDPKKYGGFDSPTVAYSVL | |
| | | VVAKVEKGKSKKLKSVKELLGITIMERSSFEKNPIDFLEAKGYK | |
| | | EVKKDLIIKLPKYSLFELENGRKRMLASAGELQKGNELALPSKY | |
| | | VNFLYLASHYEKLKGSPEDNEQKQLFVEQHKHYLDEIIEQISEF | |
| | | SKRVILADANLDKVLSAYNKHRDKPIREQAENIIHLFTLTNLGA | |
| | | PAAFKYFDTTIDRKRYTSTKEVLDATLIHQSITGLYETRIDLSQ | |
| | | LGGD | |
| 12 | Protein | MDKKYSIGLAIGTNSVGWAVITDEYKVPSKKFKVLGNTDRHSIK | S. Pyogenes Cas9 |
| | | KNLIGALLFDSGETAEATRLKRTARRRYTRRKNRICYLQEIFSN | D10A |
| | | EMAKVDDSFEHRLEESELVEEDKKHERHPIFGNIVDEVAYHEKY | |
| | | PTIYHLRKKLVDSTDKADLRLIYLALAHMIKERGHFLIEGDLNP | |
| | | DNSDVDKLFIQLVQTYNQLFEENPINASGVDAKAILSARLSKSR | |
| | | RLENLIAQLPGEKKNGLFGNLIALSLGLTPNEKSNFDLAEDAKL | |
| | | QLSKDTYDDDLDNLLAQIGDQYADLFLAAKNLSDAILLSDILRV | |
| | | NTEITKAPLSASMIKRYDEHHQDLTLLKALVRQQLPEKYKEIFF | |
| | | DQSKNGYAGYIDGGASQEEFYKFIKPILEKMDGTEELLVKLNRE | |
| | | DLLRKQRTFDNGSIPHQIHLGELHAILRRQEDFYPFLKDNREKI | |
| | | EKILTFRIPYYVGPLARGNSRFAWMTRKSEETITPWNFEEVVDK | |
| | | GASAQSFIERMTNEDKNLPNEKVLPKHSLLYEYFTVYNELTKVK | |
| | | YVTEGMRKPAELSGEQKKAIVDLLEKTNRKVTVKQLKEDYFKKI | |
| | | ECFDSVEISGVEDRFNASLGTYHDLLKIIKDKDFLDNEENEDIL | |
| | | EDIVLTLTLFEDREMIEERLKTYAHLFDDKVMKQLKRRRYTGWG | |
| | | RLSRKLINGIRDKQSGKTILDFLKSDGFANRNFMQLIHDDSLTF | |
| | | KEDIQKAQVSGQGDSLHEHIANLAGSPAIKKGILQTVKVVDELV | |
| | | KVMGRHKPENIVIEMARENQTTQKGQKNSRERMKRIEEGIKELG | |
| | | SQILKEHPVENTQLQNEKLYLYYLQNGRDMYVDQELDINRLSDY | |
| | | DVDHIVPQSFLKDDSIDNKVLTRSDKNRGKSDNVPSEEVVKKMK | |
| | | NYWRQLLNAKLITQRKFDNLTKAERGGLSELDKAGFIKRQLVET | |
| | | RQITKHVAQILDSRMNTKYDENDKLIREVKVITLKSKLVSDFRK | |
| | | DFQFYKVREINNYHHAHDAYLNAVVGTALIKKYPKLESEFVYGD | |
| | | YKVYDVRKMIAKSEQEIGKATAKYFFYSNIMNFFKTEITLANGE | |
| | | IRKRPLIETNGETGEIVWDKGRDFATVRKVLSMPQVNIVKKTEV | |
| | | QTGGFSKESILPKRNSDKLIARKKDWDPKKYGGFDSPTVAYSVL | |
| | | VVAKVEKGKSKKLKSVKELLGITIMERSSFEKNPIDFLEAKGYK | |
| | | EVKKDLIIKLPKYSLFELENGRKRMLASAGELQKGNELALPSKY | |
| | | VNFLYLASHYEKLKGSPEDNEQKQLFVEQHKHYLDEIIEQISEF | |
| | | SKRVILADANLDKVLSAYNKHRDKPIREQAENIIHLFTLTNLGA | |
| | | PAAFKYFDTTIDRKRYTSTKEVLDATLIHQSITGLYETRIDLSQ | |
| | | LGGD | |
| 13 | DNA | TGACGTTACCTCGTGCGGCC | PDCD1 CRISPR guide |
| | | | RNA target sequence 1 |
| 14 | DNA | CACGAAGCTCTCCGATGTGT | PDCD1 CRISPR guide |
| | | | RNA target sequence 2 |
| 15 | DNA | GCGTGACTTCCACATGAGCG | PDCD1 CRISPR guide |
| | | | RNA target sequence 3 |
| 16 | DNA | TTGGAACTGGCCGGCTGGCC | PDCD1 CRISPR guide |
| | | | RNA target sequence 4 |
| 17 | DNA | GTGGCATACTCCGTCTGCTC | PDCD1 CRISPR guide |
| | | | RNA target sequence 5 |
| 18 | DNA | GATGAGGTGCCCATTCCGCT | PDCD1 CRISPR guide |
| | | | RNA target sequence 6 |
| 19 | DNA | TACCGCTGCATGATCAGCTA | CD274 CRISPR guide |
| | | | RNA target sequence 1 |
| 20 | DNA | AGCTACTATGCTGAACCTTC | CD274 CRISPR guide |
| | | | RNA target sequence 2 |
| 21 | DNA | GGATGACCAATTCAGCTGTA | CD274 CRISPR guide |
| | | | RNA target sequence 3 |
| 22 | DNA | ACCCCAAGGCCGAAGTCATC | CD274 CRISPR guide |
| | | | RNA target sequence 4 |
| 23 | DNA | TCTTTATATTCATGACCTAC | CD274 CRISPR guide |
| | | | RNA target sequence 5 |
| 24 | DNA | ACCGTTCAGCAAATGCCAGT | CD274 CRISPR guide |
| | | | RNA target sequence 6 |

1. An engineered T cell, comprising:
(a) a genetically engineered antigen receptor that specifically binds to an antigen; and
(b) an inhibitory nucleic acid molecule that reduces, or is capable of effecting reduction of, expression of PD-L1.

2. The cell of claim 1, wherein the inhibitory nucleic acid molecule comprises an RNA interfering agent.

3. The cell of claim 1 or claim 2, wherein the inhibitory nucleic acid is or comprises or encodes a small interfering RNA (siRNA), a microRNA-adapted shRNA, a short hairpin RNA (shRNA), a hairpin siRNA, a precursor microRNA (pre-miRNA) or a microRNA (miRNA).

4. The cell of any of claims 1-3, wherein the inhibitory nucleic acid molecule comprises a sequence complementary to a PD-L1-encoding nucleic acid.

5. The cell of claim 1, wherein the inhibitory nucleic acid molecule comprises an antisense oligonucleotide complementary to a PD-L1-encoding nucleic acid.

6. A genetically engineered T cell, comprising:
(a) a genetically engineered antigen receptor that specifically binds to an antigen; and
(b) a disrupted gene encoding a PD-L1, an agent for disruption of a gene encoding a PD-L1, and/or disruption of a gene encoding PD-L1.

7. The cell of claim 6, wherein disruption of the gene is mediated by a gene editing nuclease, a zinc finger nuclease (ZFN), a clustered regularly interspaced short palindromic nucleic acid (CRISPR)/Cas9, and/or a TAL-effector nuclease (TALEN).

8. The cell of claim 6 or claim 7, wherein the disruption comprises a deletion of at least a portion of at least one exon of the gene.

9. The cell of any of claims 6-8, wherein:
the disruption comprises a deletion, mutation, and/or insertion in the gene resulting in the presence of a premature stop codon in the gene; and/or
the disruption comprises a deletion, mutation, and/or insertion within a first or second exon of the gene.

10. The cell of any of claims 1-9, wherein expression of PD-L1 in the T cell is reduced by at least 50, 60, 70, 80, 90, or 95% as compared to the expression in the T cell in the absence of the agent or gene disruption or in the absence of activation of the T cell.

11. A genetically engineered T cell, comprising:
(a) a genetically engineered antigen receptor that specifically binds to an antigen; and
(b) a polynucleotide encoding one or more molecule(s) that reduces or disrupts expression of PD-1 or PD-L1 in the cell, wherein expression or activity of the polynucleotide is conditional.

12. The cell of claim 11, wherein the expression is under the control of a conditional promoter or enhancer or transactivator.

13. The cell of claim 12, wherein the conditional promoter or enhancer or transactivator is an inducible promoter, enhancer, or transactivator or a repressible promoter, enhancer, or transactivator.

14. The genetically engineered T cell of any of claims 11-13, wherein the molecule that reduces or disrupts expression of PD-1 or PD-L1 is or comprises or encodes an antisense molecule, siRNA, shRNA, miRNA, a gene editing nuclease, zinc finger nuclease protein (ZFN), a TAL-effector nuclease (TALEN) or a CRISPR-Cas9 combination that specifically binds to, recognizes, or hybridizes to the gene.

15. The cell of any of claims 12-14, wherein the promoter is selected from among an RNA pol I, pol II or pol III promoter.

16. The cell of claim 15, wherein the promoter is selected from:
a pol III promoter that is a U6 or H1 promoter; or
a pol II promoter that is a CMV, SV40 early region or adenovirus major late promoter.

17. The cell of any of claims 12-16, wherein the promoter is an inducible promoter.

18. The cell of claim 17, wherein the promoter comprises a Lac operator sequence, a tetracycline operator sequence, a galactose operator sequence or a doxycycline operator sequence, or is an analog thereof.

19. The cell of any of claims 12-16, wherein the promoter is a repressible promoter.

20. The cell of claim 19, wherein the promoter comprises a Lac repressible element or a tetracycline repressible element, or is an analog thereof.

21. The cell of any of claims 1-20, wherein the T cell is a CD4+ or CD8+ T cell.

22. The cell of any of claims 1-21, wherein the genetically engineered antigen receptor is a functional non-T cell receptor.

23. The cell of any of claims 1-22, wherein the genetically engineered antigen receptor is a chimeric antigen receptor (CAR).

24. The cell of claim 23, wherein the CAR comprises an extracellular antigen-recognition domain that specifically binds to the antigen and an intracellular signaling domain comprising an ITAM.

25. The cell of claim 24, wherein the intracellular signaling domain comprises an intracellular domain of a CD3-zeta (CD3ζ) chain.

26. The cell of claim 24 or claim 25, wherein the CAR further comprises a costimulatory signaling region.

27. The cell of claim 26, wherein the costimulatory signaling region comprises a signaling domain of CD28 or 4-1BB.

28. The cell of claim 26 or claim 27, wherein the costimulatory signaling region is a signaling domain of CD28.

29. The cell of any of claims 1-28 that is a human cell.

30. The cell of any of claims 1-29 that is an isolated cell.

31. A nucleic acid molecule, comprising a first nucleic acid, which is optionally a first expression cassette, encoding an antigen receptor (CAR) and a second nucleic acid, which is optionally a second expression cassette, encoding an inhibitory nucleic acid molecule against PD-1 or PD-L1.

32. The nucleic acid molecule of claim 31, wherein the inhibitory nucleic acid molecule comprises an RNA interfering agent.

33. The nucleic acid molecule of claim 31 or claim 32, wherein the inhibitory nucleic acid molecule is or comprises or encodes a small interfering RNA (siRNA), a microRNA-adapted shRNA, a short hairpin RNA (shRNA), a hairpin siRNA, a precursor microRNA (pre-miRNA) or a microRNA (miRNA).

34. The nucleic acid molecule of any of claims 31-33, wherein the inhibitory nucleic acid molecule comprises a sequence complementary to a PD-L1-encoding nucleic acid.

35. The nucleic acid molecule of claim 31, wherein the inhibitory nucleic acid molecule comprises an antisense oligonucleotide complementary to a PD-L1-encoding nucleic acid.

36. The nucleic acid molecule of any of claims 31-35, wherein the antigen receptor is a functional non-T cell receptor.

37. The nucleic acid molecule of any of claims 31-36, wherein the genetically engineered antigen receptor is a chimeric antigen receptor (CAR).

38. The nucleic acid molecule of claim 37, wherein the CAR comprises an extracellular antigen-recognition domain that specifically binds to the antigen and an intracellular signaling domain comprising an ITAM.

39. The nucleic acid molecule of claim 38, wherein the intracellular signaling domain comprises an intracellular domain of a CD3-zeta (CD3ζ) chain.

40. The nucleic acid molecule of claim 38 or claim 39, wherein the CAR further comprises a costimulatory signaling region.

41. The nucleic acid molecule of claim 40, wherein the costimulatory signaling region comprises a signaling domain of CD28 or 4-1BB.

42. The nucleic acid molecule of claim 40 or claim 41, wherein the costimulatory signaling region is a signaling domain of CD28.

43. The nucleic acid molecule of any of claims 31-42, wherein the first and second nucleic acids, optionally the first and second expression cassettes, are operably linked to the same or different promoters.

44. The nucleic acid molecule of any of claims 31-43, wherein the first nucleic acid, optionally first expression cassette, is operably linked to an inducible promoter or a repressible promoter and the second nucleic acid, optionally second expression cassette, is operably linked to a constitutive promoter.

45. The nucleic acid molecule of any of claims 31-44 that is isolated.

46. A vector, comprising the nucleic acid molecule of any of claims 31-45.

47. The vector of claim 46, wherein the vector is a plasmid, lentiviral vector, retroviral vector, adenoviral vector, or adeno-associated viral vector.

48. The vector of claim 47 that is integrase defective.

49. A T cell, comprising the nucleic acid molecule of any of claims 31-45 or vector of any of claims 46-48.

50. The T cell of claim 49 that is a CD4+ or CD8+ T cell.

51. The T cell of claim 49 or claim 50 that is a human cell.

52. The T cell of any of claims 49-51 that is isolated.

53. A pharmaceutical composition, comprising the cell of any of claim 1-30 or 49-52 and a pharmaceutically acceptable carrier.

54. A method of producing a genetically engineered T cell, comprising:
(a) introducing a genetically engineered antigen receptor that specifically binds to an antigen into a population of cells comprising T cells; and
(b) introducing into the population of cells an agent capable of leading to a reduction of expression of PD-L1 and/or inhibiting upregulation of PD-L1 in T cells in the population upon incubation under one or more conditions, as compared to PD-L1 expression and/or upregulation in T cells in a corresponding population of cells not introduced with the agent upon incubation under the one or more conditions,
wherein steps (a) and (b) are carried out simultaneously or sequentially in any order, thereby introducing the genetically engineered antigen receptor and the agent into a T cell in the population.

55. A method of regulating expression of PD-L1 in a genetically engineered T cell, comprising introducing into a T cell an agent capable of leading to a reduction of expression of PD-L1 and/or inhibiting upregulation of PD-L1 in the cell upon incubation under one or more conditions, as compared to expression or upregulation of PD-L1 in a corresponding T cell not introduced with the agent upon incubation under the one or more conditions, said T cell comprising a genetically engineered antigen receptor that specifically binds to an antigen.

56. The method of claim 54 or claim 55, wherein incubation under conditions comprising the presence of antigen induces expression or upregulation of PD-L1 in the corresponding population comprising T cells not introduced with the agent.

57. The method of claim 56, wherein the incubation in the presence of antigen comprises incubating the cells in vitro with the antigen.

58. The method of claim 57, wherein the incubation in the presence of antigen is for 2 hours to 48 hours, 6 hours to 30 hours or 12 hours to 24 hours, each inclusive, or is for less than 48 hours, less than 36 hours or less than 24 hours.

59. The method of claim 56, wherein the incubation comprises administration of the cells to a subject under conditions whereby the engineered antigen receptor specifically binds to the antigen for at least a portion of the incubation.

60. The method of claim 59, wherein the incubation induces expression or upregulation within a period of 24 hours, 2 days, 3 days, 4 days, 5 days, 6 days, 7 days, 8 days, 9 days or 10 days following administration of cells to the subject.

61. The method of any of claims 54-60, wherein the reduction in expression or inhibition of upregulation of PD-L1 is by at least or at least about 30%, 40%, 50%, 60%, 70%, 80%, 90%, 95% or more.

62. The method of any of claims 54-61 that is performed ex vivo.

63. The method of any of claims 54-62, wherein the introducing the agent is carried out by introducing a nucleic acid comprising a sequence encoding the agent.

64. The method of any of claims 54-63, wherein the introducing comprises inducing transient expression of the agent in the T cell to effect temporary reduction or disruption of expression of PD-L1 in the cell, and/or wherein the reduction or disruption is not permanent.

65. The method of any of claims 54-64, wherein expression or activity of the agent is conditional.

66. The method of claim 65, wherein the expression is under the control of a conditional promoter or enhancer or transactivator.

67. The method of claim 66, wherein the conditional promoter or enhancer or transactivator is an inducible promoter, enhancer or transactivator or a repressible promoter, enhancer or transactivator.

68. The method of claim 66 or claim 67, wherein the promoter is selected from an RNA pol I, pol II or pol III promoter.

69. The method of claim 68, wherein the promoter is selected from:
a pol III promoter that is a U6 or an H1 promoter; or
a pol II promoter that is a CMV, a SV40 early region or an adenovirus major late promoter.

70. The method of any of claims 66-69, wherein the promoter is an inducible promoter.

71. The method of claim 70, wherein the promoter comprises a Lac operator sequence, a tetracycline operator sequence, a galactose operator sequence or a doxycycline operator sequence.

72. The method of any of claims 66-69, wherein the promoter is a repressible promoter.

73. The method of claim 72, wherein the promoter comprises a Lac repressible element or a tetracycline repressible element.

74. The method of any of claims 54-63, wherein the agent is stably expressed in the T cell to effect continued reduction or disruption of expression of PD-L1 in the cell.

75. The method of any of claims 54-74, wherein the agent is a nucleic acid molecule that is contained in a viral vector.

76. The method of claim 75, wherein the viral vector is an adenovirus, lentivirus, retrovirus, herpesvirus or adeno-associated virus vector.

77. The method of any of claims 54-76, wherein the agent is an inhibitory nucleic acid molecule that reduces expression of PD-L1 in the cell.

78. The method of claim 77, wherein the inhibitory nucleic acid molecule comprises an RNA interfering agent.

79. The method of claim 77 or claim 78, wherein the inhibitory nucleic acid is or comprises or encodes a small interfering RNA (siRNA), a microRNA-adapted shRNA, a short hairpin RNA (shRNA), a hairpin siRNA, a precursor microRNA (pre-miRNA) or a microRNA (miRNA).

80. The method of any of claim 78 or claim 79, wherein the inhibitory nucleic acid molecule comprises a sequence complementary to a PD-L1-encoding nucleic acid.

81. The method of claim 77, wherein the inhibitory nucleic acid molecule comprises an antisense oligonucleotide complementary to a PD-L1-encoding nucleic acid.

82. The method of any of claims 54-81, wherein the effecting reduction and/or inhibiting upregulation comprises disrupting a gene encoding PD-L1.

83. The method of claim 82, wherein:
the disruption comprises disrupting the gene at the DNA level and/or
the disruption is not reversible; and/or
the disruption is not transient.

84. The method of claim 82 or 83, wherein the disruption comprises introducing a DNA binding protein or DNA-binding nucleic acid that specifically binds to or hybridizes to the gene.

85. The method of claim 84, wherein the disruption comprises introducing: (i) a fusion protein comprising a DNA-targeting protein and a nuclease or (ii) an RNA-guided nuclease.

86. The method of claim 85, wherein the DNA-targeting protein or RNA-guided nuclease comprises a zinc finger protein (ZFP), a TAL protein, or a clustered regularly interspaced short palindromic nucleic acid (CRISPR) specific for the gene.

87. The method of any of claims 82-86, wherein the disruption comprises introducing a zinc finger nuclease (ZFN), a TAL-effector nuclease (TALEN), or a CRISPR-Cas9 combination that specifically binds to, recognizes, or hybridizes to the gene.

88. The method of any of claims 84-87, wherein the introducing is carried out by introducing a nucleic acid comprising a sequence encoding the DNA-binding protein, DNA-binding nucleic acid, and/or complex comprising the DNA-binding protein or DNA-binding nucleic acid.

89. The method of claim 88, wherein the nucleic acid is in a viral vector.

90. The method of any of claims 84-89, wherein the specific binding to the gene is within an exon of the gene and/or is within a portion of the gene encoding an N-terminus of the encoded polypeptide.

91. The method of any of claims 84-90, wherein the introduction thereby effects a frameshift mutation in the gene and/or an insertion of an early stop codon within the coding region of the gene.

92. The method of any of claims 54-91, further comprising introducing into the cell an agent capable of leading to a reduction of expression of PD-1 and/or inhibiting upregulation of PD-1 in the cell upon incubation under the one or more conditions compared to PD-1 expression or upregulation in a corresponding cell not introduced with the agent upon incubation under the one or more conditions, wherein the reduction of expression and/or inhibition of upregulation is temporary or transient.

93. The method of claim 92, wherein the agent is inducibly expressed or repressed in the cell to effect conditional reduction or disruption of expression of PD-1 in the cell.

94. A method of producing a genetically engineered T cell, comprising:
(a) introducing a genetically engineered antigen receptor that specifically binds to an antigen into a population of cells comprising T cells; and
(b) introducing into the population of cells an agent capable of transient reduction of expression of PD-1 and/or a transient inhibition of upregulation of PD-1 in T cells in the population upon incubation under one or more conditions, as compared to PD-1 expression and/or upregulation in T cells in a corresponding population of cells not introduced with the agent upon incubation under the one or more conditions,
wherein steps (a) and (b) are carried out simultaneously or sequentially in any order, thereby introducing the genetically engineered antigen receptor and the agent into a T cell in the population.

95. A method of regulating expression of PD-1 in a genetically engineered T cell, comprising introducing into a T cell an agent capable of transient reduction of expression of PD-1 and/or a transient inhibition of upregulation of PD-1 in the cell upon incubation under one or more conditions, as compared to expression or upregulation of PD-1 in a corresponding T cell not introduced with the agent upon incubation under the one or more conditions, said T cell comprising an antigen receptor that specifically binds to an antigen.

96. The method of claim 94 or claim 95, wherein transient reduction comprises reversible reduction in expression of PD-1 in the cell.

97. The method of any of claims 94-96, wherein incubation under conditions comprising the presence of antigen induces expression or upregulation of PD-1 in the corresponding population comprising T cells not introduced with the agent.

98. The method of claim 97, wherein the incubation in the presence of antigen comprises incubating the cells in vitro with the antigen.

99. The method of claim 98, wherein the incubation in the presence of antigen is for 2 hours to 48 hours, 6 hours to 30 hours or 12 hours to 24 hours, each inclusive, or is for less than 48 hours, less than 36 hours or less than 24 hours.

100. The method of claim 97, wherein the incubation comprises administration of the cells to a subject under conditions whereby the engineered antigen receptor specifically binds to the antigen for at least a portion of the incubation.

101. The method of claim 100, wherein the incubation induces expression or upregulation within a period of 24 hours, 2 days, 3 days, 4 days, 5 days, 6 days, 7 days, 8 days, 9 days or 10 days following administration of cells to the subject.

102. The method of any of claims 94-101, wherein the reduction in expression or inhibition of upregulation of PD-1 is by at least or at least about 30%, 40%, 50%, 60%, 70%, 80%, 90%, 95% or more.

103. The method of any of claims 94-102 that is performed ex vivo.

104. The method of any of claims 94-103, wherein the introducing in (b) is carried out by introducing into the cell a nucleic acid comprising a sequence encoding the agent.

105. The method of any of claims 94-104, wherein the agent is transiently expressed in the cell to effect temporary reduction or disruption of expression of PD-1 in the T cell.

106. The method of any of claims 94-105, wherein the expression or activity of the agent is conditional.

107. The method of claim 106, wherein the expression is under the control of a conditional promoter or enhancer or transactivator.

108. The method of claim 107, wherein the conditional promoter or enhancer or transactivator is an inducible promoter, enhancer or transactivator is a repressible promoter, enhancer or transactivator.

109. The method of claim 108, wherein the promoter is selected from an RNA pol I, pol II or pol III promoter.

110. The method of claim 109, wherein the promoter is selected from:
a pol III promoter that is a U6 or an H1 promoter; or
a pol II promoter that is a CMV, a SV40 early region or an adenovirus major late promoter.

111. The method of any of claims 108-110, wherein the promoter is an inducible promoter.

112. The method of claim 111, wherein the promoter comprises a Lac operator sequence, a tetracycline operator sequence, a galactose operator sequence or a doxycycline operator sequence.

113. The method of any of claims 108-112, wherein the promoter is a repressible promoter.

114. The method of claim 113, wherein the promoter comprises a Lac repressible element or a tetracycline repressible element.

115. The method of any of claims 92-114, wherein the agent is an inhibitory nucleic acid molecule that reduces expression of PD-1 in the cell.

116. The method of claim 115, wherein the inhibitory nucleic acid molecule comprises an RNA interfering agent.

117. The method of claim 115 or claim 116, wherein the inhibitory nucleic acid is or comprises or encodes a small interfering RNA (siRNA), a microRNA-adapted shRNA, a short hairpin RNA (shRNA), a hairpin siRNA, a precursor microRNA (pre-miRNA) or a microRNA (miRNA).

118. The method of any of claims 115-117, wherein the inhibitory nucleic acid molecule comprises a sequence complementary to a PD-1-encoding nucleic acid.

119. The method of claim 115, wherein the inhibitory nucleic acid molecule comprises an antisense oligonucleotide complementary to a PD-1-encoding nucleic acid.

120. The method of any of claims 54-119, wherein the T cell is a CD4+ or CD8+ T cell.

121. The method of any of claims 54-120, wherein the genetically engineered antigen receptor is a functional non-T cell receptor.

122. The method of any of claims 54-121, wherein the genetically engineered antigen receptor is a chimeric antigen receptor (CAR).

123. The method of claim 122, wherein the CAR comprises an extracellular antigen-recognition domain that specifically binds to the antigen and an intracellular signaling domain comprising an ITAM.

124. The method of claim 123, wherein the intracellular signaling domain comprises an intracellular domain of a CD3-zeta (CD3ζ) chain.

125. The method of claim 123 or claim 124, wherein the CAR further comprises a costimulatory signaling region.

126. The method of claim 125, wherein the costimulatory signaling region comprises a signaling domain of CD28 or 4-1BB.

127. The method of claim 125 or claim 126, wherein the costimulatory signaling region is a signaling domain of CD28.

128. The method of claim 127, wherein the steps (a) and (b) are performed simultaneously, said steps comprising introducing a nucleic acid molecule comprising a first nucleic acid, which is optionally a first expression cassette, encoding the antigen receptor and a second nucleic acid, which is optionally a second expression cassette, encoding the agent to effect reduction of expression of PD-1 or PD-L1.

129. The method of claim 127 or claim 128, further comprising introducing into the population of cells a nucleic acid molecule encoding a second genetically engineered antigen receptor that specifically binds to the same or a different antigen, said second antigen receptor comprising a costimulatory signaling region other than a signaling domain of CD28.

130. A method of producing a genetically engineered T cell, comprising:
(a) introducing a first genetically engineered antigen receptor that specifically binds to a first antigen into a population of cells comprising T cells, said first antigen receptor comprising a CD28 costimulatory signaling domain;
(b) introducing into the population of cells comprising T cells a nucleic acid molecule encoding a second genetically engineered antigen receptor that specifically binds to the same or different antigen; and
(c) introducing into the population of cells comprising T cells an agent capable of leading to a reduction of expression of PD-1 or PD-L1 and/or inhibiting upregulation of PD-1 or PD-L1 in T cells in the population upon incubation under one or more conditions, as compared to PD-1 and/or PD-L1 expression or upregulation in T cells in a corresponding population of cells not introduced with the agent upon incubation under the one or more conditions, thereby introducing the first antigen receptor, the second antigen receptor and the agent into a T cell in the population.

131. The method of claim 130, wherein incubation under conditions comprising the presence of antigen induces expression or upregulation of PD-1 and/or PD-L1 in the corresponding population comprising T cells not introduced with the agent.

132. The method of claim 131, wherein the incubation in the presence of antigen comprises incubating the cells in vitro with the antigen.

133. The method of claim 132, wherein the incubation in the presence of antigen is for 2 hours to 48 hours, 6 hours to 30 hours or 12 hours to 24 hours, each inclusive, or is for less than 48 hours, less than 36 hours or less than 24 hours.

134. The method of claim 131, wherein the incubation comprises administration of the cells to a subject under conditions whereby the engineered antigen receptor specifically binds to the antigen for at least a portion of the incubation.

135. The method of claim 134, wherein the incubation induces expression or upregulation within a period of 24 hours, 2 days, 3 days, 4 days, 5 days, 6 days, 7 days, 8 days, 9 days or 10 days following administration of cells to the subject.

136. The method of any of claims 130-135, wherein expression or upregulation of PD-1 and/or PD-L1 in the cells in inhibited or reduced by at least or at least about 30%, 40%, 50%, 60%, 70%, 80%, 90%, 95% or more compared to an engineered cell produced by the method in the absence of introducing the agent.

137. The method of any of claims 129-136, wherein the first and second genetically engineered antigen receptor bind the same antigen.

138. The method of any of claims 130-137, wherein the second antigen receptor comprises a costimulatory signaling region other than a signaling domain of CD28.

139. The method of any of claims 129-138, wherein the costimulatory signaling region other than a signaling domain of CD28 is a signaling domain of 4-1BB.

140. The method of any of claims 130-139, wherein the agent effects reduction of expression and/or inhibition of upregulation of PD-L1.

141. The method of any of claims 130-140, wherein steps (a)-(c) are performed simultaneously in any order, said steps comprising introducing a nucleic acid molecule comprising a first nucleic acid, which is optionally a first expression cassette, encoding the first antigen receptor, a second nucleic acid, which is optionally a second expression cassette, encoding the second antigen receptor and a third nucleic acid, which is optionally a third expression cassette, encoding the agent to effect reduction of expression of PD-1 or PD-L1.

142. The method of claim 141, wherein the nucleic acids, optionally the expression cassettes, are operably linked to the same or different promoters.

143. The method of claim 141 or claim 142, wherein the first and/or second nucleic acid, optionally first and/or second expression cassette, is operably linked to an inducible promoter or a repressible promoter and the third nucleic acid, optionally third expression cassette, is operably linked to a constitutive promoter.

144. The method of any of claims 54-143 that is a human cell.

145. A method of producing a genetically engineered T cell, comprising:
(a) obtaining a population of primary cells comprising T cells;
(b) enriching for cells in the population that do not express a target antigen; and
(c) introducing into the population of cells a genetically engineered antigen receptor that specifically binds to the target antigen; thereby producing a genetically engineered T cell.

146. The method of claim 145, further comprising culturing and/or incubating the cells under stimulating conditions to effect proliferation of the cells, wherein the proliferation and/or expansion of cells is greater than in cells produced in the method but in the absence of enriching for cells that do not express the target antigen.

147. The method of claim 146, wherein proliferation and/or expansion of cells is at least or at least about 1.5-fold, 2-fold, 3-fold, 4-fold, 5-fold, 6-fold, 7-fold, 8-fold, 9-fold, 10-fold or more greater.

148. The method of any of claims 145-147, wherein enriching for cells that do not express a target antigen comprises negative selection to deplete cells expressing the target antigen or disruption of the gene encoding the target antigen in cells in the population.

149. The method of any of claims 146-148, wherein the stimulating condition comprises an agent capable of activating one or more intracellular signaling domains of one or more components of a TCR complex.

150. A cell produced by the method of any of claims 54-149.

151. A pharmaceutical composition, comprising the cell of claim 150 and a pharmaceutically acceptable carrier.

152. A method of treatment, comprising administering to a subject having a disease or condition the cell of any of claim 1-30, 49-52 or 150 or the pharmaceutical composition of claim 46 or 115.

153. The method of treatment of claim 152, wherein the cells are administered in a dosage regime comprising:
(a) administering to the subject a first dose of cells expressing a chimeric antigen receptor (CAR); and
(b) administering to the subject a consecutive dose of CAR-expressing cells, said consecutive dose being administered to the subject at a time when expression of PD-L1 is induced or upregulated on the surface of the CAR-expressing cells administered to the subject in (a) and/or said consecutive dose being administered to the subject at least 5 days after initiation of the administration in (a).

154. A method of treatment, comprising:
(a) administering to the subject a first dose of cells expressing a chimeric antigen receptor (CAR); and
(b) administering to the subject a consecutive dose of CAR-expressing cells said consecutive dose being administered to the subject at a time when expression of PD-L1 is induced or upregulated on the surface of the CAR-expressing cells administered to the subject in (a) and/or said consecutive dose being administered to the subject at least 5 days after initiation of the administration in (a).

155. The method of claim 153 or claim 154, wherein the consecutive dose of cells is administered at least or more than about 5 days after and less than about 12 days after initiation of said administration in (a)

156. The method of any of claims 153-155, wherein the number of cells administered in the first and/or second dose is between about 0.5×10⁶ cells/kg body weight of the subject and 4×10⁶ cells/kg, between about 0.75×10⁶ cells/kg and 3.0×10⁶ cells/kg or between about 1×10⁶ cells/kg and 2×10⁶ cells/kg, each inclusive.

157. The method of any of claims 152-156, wherein the genetically engineered antigen receptor specifically binds to an antigen associated with the disease or condition.

158. The method of treatment of any of claims 152-157, wherein the disease or condition is a cancer.

159. The method of any of claims 152-158, wherein the disease or condition is a leukemia or lymphoma.

160. The method of any of claims 152-159, wherein the disease or condition is acute lymphoblastic leukemia.

161. The method of any of claims 152-159, wherein the disease or condition is a non-Hodgkin lymphoma (NHL).
